(19) United States
(12) Patent Application Publication
(10) Pub. No.: US 2023/0014637 A1
(43) Pub. Date: Jan. 19, 2023

(54) Therapeutic Monocytic Lineage Cells

(71) Applicant: CREATIVE MEDICAL TECHNOLOGIES, INC., Phoenix, AZ (US)

(72) Inventors: Thomas Ichim, San Diego, CA (US); Amit Patel, Salt Lake City, UT (US)

(73) Assignee: CREATIVE MEDICAL TECHNOLOGIES, INC., Phoenix, AZ (US)

(21) Appl. No.: 17/865,881

(22) Filed: Jul. 15, 2022

Related U.S. Application Data
(60) Provisional application No. 63/223,245, filed on Jul. 19, 2021.

(51) Int. Cl. C12N 5/0775

(52) U.S. Cl. CPC C12N 5/0665; C12N 2501/115

(57) ABSTRACT

Disclosed are novel cellular populations generated by explosion of monocytic cells to conditioned media of regenerative cells. In one embodiment said regenerative cells are umbilical cord endothelial cells and said cells are pre-activated to possess enhance ability to reprogram said monocytic lineage cells. In one embodiment monocyte lineage cells are collected from leukopaks by plastic adherence and subsequently cultured in a manner to generate cells similar to M2 cells. In one embodiment said monocytic cells are cultured in a manner to generate myeloid derived suppressor cells. In one embodiment cells are generated to reducing inflammatory conditions. In another embodiment cells are generated for treatment of degenerative conditions. In another embodiment cells are generated for treatment of fibrosis.

### CROSS-REFERENCE TO RELATED APPLICATIONS

This application is a Non-Provisional application which claims the benefit of priority to U.S. Provisional Application No. 63/223,245, filed Jul. 19, 2021, which is incorporated herein in its entirety.

### FIELD OF THE INVENTION

The teachings herein relate to immune modulating cellular populations useful for immune suppression and reducing inflammatory conditions, not limited to degenerative conditions and fibrosis.

### BACKGROUND

The bone marrow is the primary site of hematopoiesis in the adult, simultaneously producing all blood borne cells including; erythrocytes, monocyte, granulocytes, and lymphocytes. Various hematopoietic growth factors have been described which are responsible for stimulating normal hematopoiesis, including stem cell factor (SCF), M-CSF, G-CSF, and GM-CSF. Similarly, there are factors which inhibit the uncontrolled proliferation of hematopoietic cells, these include TGF-beta, MIP-1-alpha, AcSDKP and pEEDCK.

In 1972, Dr. Kim Singhal was the first to describe a subset of bone marrow regulatory cells, termed natural suppressors (NS) cells. These cells, later identified as CD31+ myeloid progenitor cells, have been demonstrated to secrete immune regulatory substances. Production of such substances such as Reptimed could be increased by treatment of bone marrow cells with the hematopoietic cytokine interleukin-3, thus further indicating a regulatory role of Reptimed in the bone marrow.

NS cells have been found to be important in maintaining successful pregnancy by protecting the fetal allograft [1-5], preventing transplant rejection [6-14], and assisting tumors in avoiding immune destruction [14-19].

Studies found that NS cells are actually now called myeloid derived suppressor cells and represent the same cell type.

### SUMMARY

Preferred embodiments are directed to method of generating a monocytic lineage cell capable of suppressing inflammation, generated by; a) obtaining a monocytic population; b) culturing said monocytic cell population in media conditioned by regenerative cells; and c) optionally adding factors capable of supporting/synergizing with regenerative cells at inducing generation of inflammation suppressing myeloid cells.

Preferred methods include embodiments wherein said monocytic cell population is extracted by plastic adherence from peripheral blood mononuclear cells.

Preferred methods include embodiments wherein said peripheral blood mononuclear cells are obtained by leukopheresis.

Preferred methods include embodiments wherein said peripheral blood mononuclear cells are obtained by use of a density gradient.

Preferred methods include embodiments wherein said density gradient is percoll.

Preferred methods include embodiments wherein said density gradient is ficoll.

Preferred methods include embodiments wherein said peripheral blood is obtain from the recipient.

Preferred methods include embodiments wherein said peripheral blood is obtain from the donor.

Preferred methods include embodiments wherein said peripheral blood is obtain from a third party.

Preferred methods include embodiments wherein said peripheral blood obtained from said third party is umbilical cord blood.

Preferred methods include embodiments wherein said peripheral blood is obtained after mobilization.

Preferred methods include embodiments wherein said mobilization is increasing the content of regenerative cells.

Preferred methods include embodiments were said regenerative cells are hematopoietic stem cells.

Preferred methods include embodiments were said regenerative cells are mesenchymal stem cells.

Preferred methods include embodiments were said regenerative cells are very small embryonic like cells (VSEL).

Preferred methods include embodiments wherein said mobilization is accomplished by administration of G-CSF.

Preferred methods include embodiments wherein said mobilization is accomplished by administration of GM-CSF.

Preferred methods include embodiments wherein said mobilization is accomplished by administration of M-CSF.

Preferred methods include embodiments wherein said mobilization is accomplished by administration of flt3 ligand.

Preferred methods include embodiments wherein said mobilization is accomplished by administration of ozone gas.

Preferred methods include embodiments wherein said monocytic cell population is isolated by positive selection for cells expressing CD11c.

Preferred methods include embodiments wherein said monocytic cell population is isolated by positive selection for cells expressing CD105.

Preferred methods include embodiments wherein said monocytic cell population is isolated by positive selection for cells expressing CD14.

Preferred methods include embodiments wherein said monocytic cell population is isolated by positive selection for cells expressing CD16.

Preferred methods include embodiments wherein said monocytic cell population is isolated by positive selection for cells expressing GR-1.

Preferred methods include embodiments wherein said monocytic cell population is isolated by positive selection for cells expressing interferon gamma receptor.

Preferred methods include embodiments wherein said monocytic cell population is isolated by positive selection for cells expressing interleukin-4 receptor.

Preferred methods include embodiments wherein said monocytic cell population is isolated by positive selection for cells expressing interleukin-13 receptor.

Preferred methods include embodiments wherein said monocytic cell population is isolated by positive selection for cells expressing interleukin-10 receptor.

Preferred methods include embodiments wherein said monocytic cell population is isolated by positive selection for cells expressing HLA-DR.

Preferred methods include embodiments wherein said monocytic cell population is isolated by positive selection for cells expressing TGF-beta receptor.

Preferred methods include embodiments wherein inflammation is associated with elevated circulating levels of a cytokine capable of activating NF-kappa B as compared to an age matched control.

Preferred methods include embodiments wherein inflammation is a condition in which elevated concentrations of interleukin-1 are found in comparison to an age-matched control.

Preferred methods include embodiments wherein inflammation is a condition in which elevated concentrations of interleukin-2 are found in comparison to an age-matched control.

Preferred methods include embodiments wherein inflammation is a condition in which elevated concentrations of interleukin-3 are found in comparison to an age-matched control.

Preferred methods include embodiments wherein inflammation is a condition in which elevated concentrations of interleukin-5 are found in comparison to an age-matched control.

Preferred methods include embodiments wherein inflammation is a condition in which elevated concentrations of interleukin-6 are found in comparison to an age-matched control.

Preferred methods include embodiments wherein inflammation is a condition in which elevated concentrations of interleukin-7 are found in comparison to an age-matched control.

Preferred methods include embodiments wherein inflammation is a condition in which elevated concentrations of interleukin-8 are found in comparison to an age-matched control.

Preferred methods include embodiments wherein inflammation is a condition in which elevated concentrations of interleukin-9 are found in comparison to an age-matched control.

Preferred methods include embodiments wherein inflammation is a condition in which elevated concentrations of interleukin-11 are found in comparison to an age-matched control.

Preferred methods include embodiments wherein inflammation is a condition in which elevated concentrations of interleukin-12 are found in comparison to an age-matched control.

Preferred methods include embodiments wherein inflammation is a condition in which elevated concentrations of interleukin-15 are found in comparison to an age-matched control.

Preferred methods include embodiments wherein inflammation is a condition in which elevated concentrations of interleukin-17 are found in comparison to an age-matched control.

Preferred methods include embodiments wherein inflammation is a condition in which elevated concentrations of interleukin-18 are found in comparison to an age-matched control.

Preferred methods include embodiments wherein inflammation is a condition in which elevated concentrations of interleukin-21 are found in comparison to an age-matched control.

Preferred methods include embodiments wherein inflammation is a condition in which elevated concentrations of interleukin-23 are found in comparison to an age-matched control.

Preferred methods include embodiments wherein inflammation is a condition in which elevated concentrations of interleukin-27 are found in comparison to an age-matched control.

Preferred methods include embodiments wherein inflammation is a condition in which elevated concentrations of interleukin-33 are found in comparison to an age-matched control.

Preferred methods include embodiments wherein inflammation is a condition in which elevated concentrations of TNF-alpha are found in comparison to an age-matched control.

Preferred methods include embodiments wherein inflammation is a condition in which elevated concentrations of TNF-beta are found in comparison to an age-matched control.

Preferred methods include embodiments wherein inflammation is a condition in which elevated concentrations of MIP-1 alpha are found in comparison to an age-matched control.

Preferred methods include embodiments wherein inflammation is a condition in which elevated concentrations of MIP-1 beta are found in comparison to an age-matched control.

Preferred methods include embodiments wherein inflammation is a condition in which elevated concentrations of macrophage activation factor (MAF) are found in comparison to an age-matched control.

Preferred methods include embodiments wherein inflammation is a condition in which elevated concentrations of interferon gamma are found in comparison to an age-matched control.

Preferred methods include embodiments wherein inflammation is a condition in which elevated concentrations of interferon alpha are found in comparison to an age-matched control.

Preferred methods include embodiments wherein inflammation is a condition in which elevated concentrations of interferon beta are found in comparison to an age-matched control.

Preferred methods include embodiments wherein inflammation is a condition in which elevated concentrations of interferon omega are found in comparison to an age-matched control.

Preferred methods include embodiments wherein inflammation is a condition in which elevated concentrations of interferon tau are found in comparison to an age-matched control.

Preferred methods include embodiments wherein said inflammation is an autoimmune condition.

Preferred methods include embodiments wherein said autoimmune condition is selected from a group consisting of: Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticaria, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myelin Oligodendrocyte Glycoprotein Antibody Disorder, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary Biliary Cholangitis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, and Vogt-Koyanagi-Harada Disease

Preferred methods include embodiments wherein said regenerative cells are stem cells.

Preferred methods include embodiments wherein said stem cells are pluripotent stem cells.

Preferred methods include embodiments wherein said pluripotent stem cells possess ability to generate all major tissues of the body.

Preferred methods include embodiments wherein said pluripotent stem cells express SSEA 3.

Preferred methods include embodiments wherein said pluripotent stem cells express SSEA 4.

Preferred methods include embodiments wherein said pluripotent stem cells express Tra-1-60.

Preferred methods include embodiments wherein said pluripotent stem cells express Tra-1-81.

Preferred methods include embodiments wherein said pluripotent stem cells express Oct-3/4.

Preferred methods include embodiments wherein said pluripotent stem cells express Cripto.

Preferred methods include embodiments wherein said pluripotent stem cells express gastrin-releasing peptide (GRP) receptor.

Preferred methods include embodiments wherein said pluripotent stem cells express podocalyxin-like protein (PODXL).

Preferred methods include embodiments wherein said pluripotent stem cells express Rex-1.

Preferred methods include embodiments wherein said pluripotent stem cells express GCTM-2.

Preferred methods include embodiments wherein said pluripotent stem cells express Nanog.

Preferred methods include embodiments wherein said pluripotent stem cells express SOX-2.

Preferred methods include embodiments wherein said pluripotent stem cells express KLF-1.

Preferred methods include embodiments wherein said pluripotent stem cells express human telomerase reverse transcriptase (hTERT).

Preferred methods include embodiments wherein said pluripotent stem cells are selected for higher expression of SOX-2 as compared to hematopoietic stem cells.

Preferred methods include embodiments wherein said pluripotent stem cells are selected for higher expression of KLF-1 as compared to hematopoietic stem cells.

Preferred methods include embodiments wherein said pluripotent stem cells are selected for higher expression of NANOG as compared to hematopoietic stem cells.

Preferred methods include embodiments wherein said pluripotent stem cells are selected for higher expression of IL-3 receptor as compared to hematopoietic stem cells.

Preferred methods include embodiments wherein said pluripotent stem cells are selected for higher expression of IL-7 receptor as compared to hematopoietic stem cells.

Preferred methods include embodiments wherein said pluripotent stem cells are selected for higher expression of IL-10 receptor as compared to hematopoietic stem cells.

Preferred methods include embodiments wherein said pluripotent stem cells are selected for higher expression of BMP-2 receptor as compared to hematopoietic stem cells.

Preferred methods include embodiments wherein said pluripotent stem cells are selected for higher expression of BMP-4 receptor as compared to hematopoietic stem cells.

Preferred methods include embodiments wherein said pluripotent stem cells are selected for higher expression of TGF-beta receptor as compared to hematopoietic stem cells.

Preferred methods include embodiments wherein said stem cell is selected from a group comprising of a) embryonic stem cells; b) hematopoietic stem cells; c) mesenchymal stem cells; d) very small embryonic like stem cells; e) inducible pluripotent stem cells; f) bone marrow stem cells; g) amniotic fluid stem cells; h) neuronal stem cells; i) parthenogenically derived stem cells; j) cord blood stem cells; k) placental stem cells; 1) bone marrow stem cells; m) germinal stem cells; n) hair follicle stem cells; o) adipose derived stem cells; p) reprogrammed stem cells; q) peripheral blood derived stem cells; r) peripheral blood mesenchymal stem cells; s) endometrial regenerative cells; t) fallopian tube derived stem cells; u) dermal stem cells; v) side population stem cells; and w) subepithelial umbilical cord tissue stem cells.

Preferred methods include embodiments wherein said embryonic stem cells are totipotent.

Preferred methods include embodiments wherein said embryonic stem cells express stage-specific embryonic antigens (SSEA) 3.

Preferred methods include embodiments wherein said embryonic stem cells express stage-specific embryonic antigens (SSEA) 4.

Preferred methods include embodiments wherein said embryonic stem cells express Tra-1-60.

Preferred methods include embodiments wherein said embryonic stem cells express Tra-1-81.

Preferred methods include embodiments wherein said embryonic stem cells express Oct-3.

Preferred methods include embodiments wherein said embryonic stem cells express Oct-4.

Preferred methods include embodiments wherein said embryonic stem cells express SOX-2.

Preferred methods include embodiments wherein said embryonic stem cells express IL-17 receptor.

Preferred methods include embodiments wherein said cord blood stem cells are multipotent and capable of differentiating into hematopoietic and mesenchymal lineages.

Preferred methods include embodiments wherein cord blood stem cells are capable of differentiating along the mesenchymal lineage.

Preferred methods include embodiments wherein said cord blood hematopoietic stem cell expresses CD34.

Preferred methods include embodiments wherein said cord blood hematopoietic stem cell expresses CD133.

Preferred methods include embodiments wherein said cord blood hematopoietic stem cell expresses flk-2.

Preferred methods include embodiments wherein said cord blood hematopoietic stem cell expresses interleukin-3 receptor.

Preferred methods include embodiments wherein said cord blood hematopoietic stem cell expresses c-met.

Preferred methods include embodiments wherein said cord blood hematopoietic stem cell expresses interleukin 6 receptor.

Preferred methods include embodiments wherein said cord blood hematopoietic stem cell expresses interleukin 8 receptor.

Preferred methods include embodiments wherein said cord blood hematopoietic stem cell expresses interleukin-11 receptor.

Preferred methods include embodiments wherein said cord blood hematopoietic stem cell expresses follistatin receptor.

Preferred methods include embodiments wherein said cord blood hematopoietic stem cell expresses TPO receptor.

Preferred methods include embodiments wherein said cord blood hematopoietic stem cell expresses BMP-2 receptor.

Preferred methods include embodiments herein said cord blood mesenchymal stem cell expresses CD90.

Preferred methods include embodiments herein said cord blood mesenchymal stem cell expresses CD105.

Preferred methods include embodiments herein said cord blood mesenchymal stem cell expresses Her2.

Preferred methods include embodiments herein said cord blood mesenchymal stem cell expresses FGF-1 receptor.

Preferred methods include embodiments herein said cord blood mesenchymal stem cell expresses FGF-2 receptor.

Preferred methods include embodiments herein said cord blood mesenchymal stem cell expresses EGF-1 receptor.

Preferred methods include embodiments herein said cord blood mesenchymal stem cell expresses IGF-1 receptor.

Preferred methods include embodiments herein said cord blood mesenchymal stem cell expresses c-met.

Preferred methods include embodiments herein said cord blood mesenchymal stem cell expresses Mg-SOD at approximately 2-fold higher than a fibroblast cell.

Preferred methods include embodiments wherein said cord blood stem cells express SSEA-3.

Preferred methods include embodiments wherein said cord blood stem cells express SSEA-4.

Preferred methods include embodiments wherein said cord blood stem cells express CD9.

Preferred methods include embodiments wherein said cord blood stem cells express CD34.

Preferred methods include embodiments wherein said cord blood stem cells express CD-133.

Preferred methods include embodiments wherein said cord blood stem cells express c-kit.

Preferred methods include embodiments wherein said cord blood stem cells express OCT-4.

Preferred methods include embodiments wherein said cord blood stem cells express NANOG.

Preferred methods include embodiments wherein said cord blood stem cells express CXCR-4.

Preferred methods include embodiments wherein said cord blood stem cells express c-met.

Preferred methods include embodiments wherein said cord blood stem cells express CD-56.

Preferred methods include embodiments wherein said cord blood stem cells express CD-57.

Preferred methods include embodiments wherein said cord blood stem cells express FoxP3.

Preferred methods include embodiments wherein said cord blood stem cells do not CD-3.

Preferred methods include embodiments wherein said cord blood stem cells do not CD-14.

Preferred methods include embodiments wherein said cord blood stem cells do not CD-16.

Preferred methods include embodiments wherein said cord blood stem cells do not CD-11b.

Preferred methods include embodiments wherein said placental stem cells are isolated from the placental structure.

Preferred methods include embodiments wherein said placental stem cells express one or more antigens selected from a group comprising: a) Oct-4; b) Rex-1; c) CD9; d) CD13; e) CD29; f) CD44; g) CD166; h) CD90; i) CD105; j) SH-3; k) SH-4; 1) TRA-1-60; m) TRA-1-81; n) SSEA-4 and; o) Sox-2.

Preferred methods include embodiments wherein said placental stem cell expresses OCT4.

Preferred methods include embodiments wherein said placental stem cell expresses Rex-1.

Preferred methods include embodiments wherein said placental stem cell expresses CD9.

Preferred methods include embodiments wherein said placental stem cell expresses CD29.

Preferred methods include embodiments wherein said placental stem cell expresses CD13.

Preferred methods include embodiments wherein said placental stem cell expresses CD44.

Preferred methods include embodiments wherein said placental stem cell expresses CD166.

Preferred methods include embodiments wherein said placental stem cell expresses CD90.

Preferred methods include embodiments wherein said placental stem cell expresses CD105.

Preferred methods include embodiments wherein said placental stem cell expresses SH3.

Preferred methods include embodiments wherein said placental stem cell expresses SH4.

Preferred methods include embodiments wherein said placental stem cell expresses TRA-1-60.

Preferred methods include embodiments wherein said placental stem cell expresses TRA-1-81.

Preferred methods include embodiments wherein said placental stem cell expresses SSEA4.

Preferred methods include embodiments wherein said placental stem cell expresses SOX-2.

Preferred methods include embodiments wherein said placental stem cell expresses IL-3 receptor.

Preferred methods include embodiments wherein said bone marrow stem cells comprise of bone marrow mononuclear cells.

Preferred methods include embodiments wherein said bone marrow mononuclear cells are isolated from bone marrow aspirate by use of a method selected from; a) gradient separation; b) erythrocyte lysis; and c) separation based on physical size.

Preferred methods include embodiments wherein said bone marrow stem cells are selected based on the ability to differentiate into mesenchymal stem cells.

Preferred methods include embodiments wherein said mesenchymal stem cells generated from said bone marrow cells are capable of differentiating into one or more cell types selected from a group comprising of; a) osteocytes; b) chondrocytes; and c) adipose tissue.

Preferred methods include embodiments wherein said bone marrow stem cells are selected based on expression of one or more of the following antigens: a) CD34; b) c-kit; c) flk-1; d) Stro-1; e) CD105; f) CD73; g) CD31; h) CD146; i) vascular endothelial-cadherin; j) CD133 and; k) CXCR-4.

Preferred methods include embodiments wherein said bone marrow stem cells express CD34.

Preferred methods include embodiments wherein said bone marrow stem cells express c-kit.

Preferred methods include embodiments wherein said bone marrow stem cells express flk-1.

Preferred methods include embodiments wherein said bone marrow stem cells express stro-1.

Preferred methods include embodiments wherein said bone marrow stem cells express CD105.

Preferred methods include embodiments wherein said bone marrow stem cells express CD73.

Preferred methods include embodiments wherein said bone marrow stem cells express CD31.

Preferred methods include embodiments wherein said bone marrow stem cells express CD146.

Preferred methods include embodiments wherein said bone marrow stem cells express vascular endothelial cadherin.

Preferred methods include embodiments wherein said bone marrow stem cells express CD133.

Preferred methods include embodiments wherein said bone marrow stem cells express CXCR-4.

Preferred methods include embodiments wherein said bone marrow stem cells are enriched for expression of CD133.

Preferred methods include embodiments wherein said amniotic fluid stem cells are isolated by introduction of a fluid extraction means into the amniotic cavity under ultrasound guidance.

Preferred methods include embodiments wherein said amniotic fluid stem cells are selected based on expression of one or more of the following antigens: a) SSEA3; b) SSEA4; c) Tra-1-60; d) Tra-1-81; e) Tra-2-54; f) HLA class I; g) CD13; h) CD44; i) CD49b; j) CD105; k) Oct-4; 1) Rex-1; m) DAZL and; n) Runx-1.

Preferred methods include embodiments wherein said amniotic fluid stem cells are selected based on lack of expression of one or more of the following antigens: a) CD34, b) CD45, and c) HLA Class II.

Preferred methods include embodiments wherein said cells possess the property of adherence to plastic and a mesenchymal-like morphology.

Preferred methods include embodiments wherein said neuronal stem cells are selected based on expression of one or more of the following antigens: a) RC-2; b) 3CB2; c) BLB; d) Sox-2hh; e) GLAST; f) Pax 6; g) nestin; h) Muashi-1; i) NCAM; j) A2B5; and k) prominin.

Preferred methods include embodiments wherein said neuronal stem cells are isolated from the dentate gyrus or the subventricular zone.

Preferred methods include embodiments wherein said circulating peripheral blood stem cells are characterized by ability to proliferate in vitro for a period of over 3 months.

Preferred methods include embodiments wherein said circulating peripheral blood stem cells are characterized by expression of a) CD34; b) CXCR4; c) CD117; d) CD113, and; e) c-met.

Preferred methods include embodiments wherein said circulating peripheral blood stem cell are expanded in vitro under conditions allowing for selective expansion of mesenchymal-like cells.

Preferred methods include embodiments wherein said circulating peripheral blood stem cells lack substantial expression of differentiation associated markers.

Preferred methods include embodiments wherein said differentiation associated markers are selected from a group comprising of a) CD2; b) CD3; c) CD4; d) CD11; e) CD11a; f) Mac-1; g) CD14; h) CD16; i) CD19; j) CD24; k) CD33; 1) CD36; m) CD38; n) CD45; o) CD56; p) CD64; q) CD68; r) CD86; s) CD66b; t) and; u) HLA-DR.

Preferred methods include embodiments wherein said mesenchymal stem cells express one or more of the following markers: a) STRO-1; b) CD105; c) CD54; d) CD106; e) HLA-I markers; f) vimentin; g) ASMA; h) collagen-1; i) fibronectin; j) LFA-3; k) ICAM-1; 1) PECAM-1; m) P-selectin; n) L-selectin; o) CD49b/CD29; p) CD49c/CD29; q) CD49d/CD29; r) CD61; s) CD18; t) CD29; u) thrombomodulin; v) telomerase; w) CD10; x) CD13; y) STRO-2; z) VCAM-1; aa) CD146 and; ab) THY-1.

Preferred methods include embodiments wherein said mesenchymal stem cells do not express substantial levels of a) HLA-DR; b) CD117; and c) CD45.

Preferred methods include embodiments wherein said mesenchymal stem cells are derived from a group selected of: a) bone marrow; b) adipose tissue; c) umbilical cord blood; d) Wharton's Jelly; e) enzymatically digested cord; f) inducible pluripotent generated cells; g) placental tissue; h) peripheral blood mononuclear cells; i) differentiated embryonic stem cells; and j) differentiated progenitor cells.

Preferred methods include embodiments wherein said germinal stem cells express markers selected from a group comprising of: a) Oct4; b) Nanog; c) Dppa5; d) Rbm; e) cyclin A2; f) Tex18; g) Stra8; h) Dazl; i) beta1- and alpha6-integrins; j) Vasa; k) Fragilis; 1) Nobox; m) c-Kit; n) Sca-1 and; o) Rex1.

Preferred methods include embodiments wherein said germinal stem cells are isolated from the testis.

Preferred methods include embodiments wherein said adipose tissue derived stem cells express markers selected from a group comprising of: a) CD13; b) CD29; c) CD44; d) CD63; e) CD73; f) CD90; g) CD166; h) Aldehyde dehydrogenase (ALDH); and i) ABCG2.

Preferred methods include embodiments wherein said adipose tissue derived stem cells are a population of purified mononuclear cells extracted from adipose tissue capable of proliferating in culture for more than 1 month.

Preferred methods include embodiments wherein said adipose tissue derived stem cells are plastic adherent.

Preferred methods include embodiments wherein said exfoliated teeth derived stem cells express markers selected from a group comprising of: a) STRO-1; b) CD146 (MUC18); c) alkaline phosphatase; d) MEPE; e) and; f) bFGF.

Preferred methods include embodiments wherein said hair follicle stem cells express markers selected from a group comprising of: a) cytokeratin 15; b) Nanog; and c) Oct-4.

Preferred methods include embodiments wherein said hair follicle stem cells are capable of proliferating in culture for a period of at least one month.

Preferred methods include embodiments wherein said hair follicle stem cells secrete one or more of the following proteins when grown in culture: a) basic fibroblast growth factor (bFGF); b) endothelin-1 (ET-1) and; c) stem cell factor (SCF).

Preferred methods include embodiments wherein said dermal stem cells express markers selected from a group comprising of: a) CD44; b) CD13; c) CD29; d) CD90; and e) CD105.

Preferred methods include embodiments wherein said dermal stem cells are capable of proliferating in culture for a period of at least one month.

Preferred methods include embodiments wherein said parthenogenically derived stem cells are generated by addition of a calcium flux inducing agent to activate an oocyte followed by enrichment of cells expressing markers selected from a group comprising of; a) SSEA-4; b) TRA 1-60; and c) TRA 1-81.

Preferred methods include embodiments wherein said reprogrammed stem cells are selected from a group comprising of: a) cells subsequent to a nuclear transfer; b) cells subsequent to a cytoplasmic transfer; c) cells treated with a DNA methyltransferase inhibitor; d) cells treated with a histone deacetylase inhibitor; e) cells treated with a GSK-3 inhibitor; f) cells induced to dedifferentiate by alteration of extracellular conditions; and g) cells treated with various combination of the mentioned treatment conditions.

Preferred methods include embodiments wherein said nuclear transfer comprises introducing nuclear material to a cell substantially enucleated, said nuclear material deriving from a host whose genetic profile is sought to be dedifferentiated.

Preferred methods include embodiments wherein said cytoplasmic transfer comprises introducing cytoplasm of a cell with a dedifferentiated phenotype into a cell with a differentiated phenotype, such that said cell with a differentiated phenotype substantially reverts to a dedifferentiated phenotype.

Preferred methods include embodiments wherein said DNA demethylating agent is selected from a group comprising of: a) 5-azacytidine; b) psammaplin A; and c) zebularine.

Preferred methods include embodiments wherein said histone deacetylase inhibitor is selected from a group comprising of: a) valproic acid; b) trichostatin-A; c) trapoxin A; and d) depsipeptide.

Preferred methods include embodiments wherein said endometrial regenerative cell is collected from menstrual blood based on selection for the marker c-kit.

Preferred methods include embodiments wherein said endometrial regenerative cell is collected from menstrual blood based on expansion of adherent cells in tissue culture.

Preferred methods include embodiments wherein said endometrial regenerative cell expresses markers selected from a group of proteins comprising of: a) CD90; b) CD105; and c) CD29.

Preferred methods include embodiments wherein said endometrial regenerative cell lacks substantial expression of markers selected from a group of proteins comprising of: a) HLA-I; b) CD14; and c) CD34.

Preferred methods include embodiments wherein said fallopian tube stem cells express markers selected from a group comprising of: a) CD29; b) CD44; c) CD90; d) CD13; e) CD73; f) SH2; g) SH3; and h) SH4.

Preferred methods include embodiments wherein said fallopian tube stem cells lacks substantial expression of markers selected from a group comprising of: a) CD34; b) CD38; c) CD45; d) CD117; e) CD133); CD31; and g) CD14.

Preferred methods include embodiments wherein said side population cells are identified based on expression multidrug resistance transport protein (ABCG2).

Preferred methods include embodiments wherein said side population cells are identified based on ability to efflux intracellular dyes.

Preferred methods include embodiments wherein said intracellular dyes may be rhodamine-123 or Hoechst 33342.

Preferred methods include embodiments wherein said side population is derived from a source of tissue, selected from; a) pancreatic tissue; b) liver tissue; c) smooth muscle tissue; d) striated muscle tissue; e) cardiac muscle tissue; f) bone tissue; g) bone marrow tissue; h) bone spongy tissue; i) cartilage tissue; j) liver tissue; k) pancreas tissue; 1) pancreatic ductal tissue; m) spleen tissue; n) thymus tissue; o) Peyer's patch tissue; p) lymph nodes tissue; q) thyroid tissue; r) epidermis tissue; s) dermis tissue; t) subcutaneous tissue; u) heart tissue; v) lung tissue; w) vascular tissue; x) endothelial tissue; y) blood cells; z) bladder tissue; aa) kidney tissue; ab) digestive tract tissue; ac) esophagus tissue; ad) stomach tissue; ae) small intestine tissue; af) large intestine tissue; ag) adipose tissue; ah) uterus tissue; ai) eye tissue; aj) lung tissue; ak) testicular tissue; al) ovarian tissue; am) prostate tissue; an) connective tissue; ao) endocrine tissue; ap) and mesentery tissue.

Preferred methods include embodiments wherein said umbilical cord derived mesenchymal cell expresses CD10.

Preferred methods include embodiments wherein said umbilical cord derived mesenchymal cell expresses CD24.

Preferred methods include embodiments wherein said umbilical cord derived mesenchymal cell expresses interleukin-11.

Preferred methods include embodiments wherein said umbilical cord derived mesenchymal cell expresses AIRE-1.

Preferred methods include embodiments wherein said umbilical cord derived mesenchymal cell expresses FoxP3.

Preferred methods include embodiments wherein said umbilical cord derived mesenchymal cell expresses angiopoietin-1.

Preferred methods include embodiments wherein said umbilical cord derived mesenchymal cell expresses CXCL1.

Preferred methods include embodiments wherein said umbilical cord derived mesenchymal cell expresses CD10 and CD73.

Preferred methods include embodiments wherein said umbilical cord derived mesenchymal cell expresses CD10 and CD133.

Preferred methods include embodiments wherein said umbilical cord derived mesenchymal cell expresses CD10 and CD90.

Preferred methods include embodiments wherein said umbilical cord derived mesenchymal cell expresses CD10 and CD274.

Preferred methods include embodiments wherein said umbilical cord derived mesenchymal cell expresses CD10 and AIRE.

Preferred methods include embodiments wherein said umbilical cord derived mesenchymal cell expresses CD10 and HLA-ABC.

Preferred methods include embodiments wherein said umbilical cord derived mesenchymal cell expresses CD10 and TLR-3.

Preferred methods include embodiments wherein said umbilical cord derived mesenchymal cell expresses CD10 and TLR-4.

Preferred methods include embodiments wherein said umbilical cord derived mesenchymal cell expresses CD10 and TLR-7.

Preferred methods include embodiments wherein said umbilical cord derived mesenchymal cell expresses CD10 and TLR-9.

Preferred methods include embodiments wherein said umbilical cord derived mesenchymal cell expresses CD10 and TLR-5.

Preferred methods include embodiments wherein said umbilical cord derived mesenchymal cell expresses CD10 and TLR-2.

Preferred methods include embodiments wherein said umbilical cord derived mesenchymal cell expresses CD10 and Stro-1.

Preferred methods include embodiments wherein mesenchymal stem cells are generated from pluripotent stem cells.

Preferred methods include embodiments wherein said mesenchymal stem cells are generated from pluripotent stem cells by a) culturing single cells in the presence of at least one growth factor in an amount sufficient to induce the differentiation of said clusters of cells into mesenchymal stem cells; b) adding one or more of basic fibroblast growth factor (bFGF), vascular endothelial growth factor (VEGF), bone morphogenic protein 4 (BMP-4), stem cell factor (SCF), Flt 3L (FL), thrombopoietin (TPO), EPO, and/or tPTD-HOXB4. The one or more of said at least one growth factor added in step (b) may be added to said culture within 36-60 hours from the start of step (a). Preferably, the one or more of said at least one growth factor added in step (b) is added to said culture within 40-48 hours from the start of step (a). The at least one factor added in step (b) may comprise one or more of bFGF, VEGF, BMP-4, SCF, FL and/or tPTD-HOXB4. The concentration of said growth factors if added in step (b) may range from about the following: bFGF is about 20-25 ng/ml, VEGF is about 20-100 ng/ml, BMP-4 is about 15-100 ng/ml, SCF is about 20-50 ng/ml, FL is about 10-50 ng/ml, TPO is about 20-50 ng/ml, and tPTD-HOXB4 is about 1.5-5 U/ml.

Preferred methods include embodiments wherein said subepithelial cord tissue stem cells are prepared by a process comprising: placing a subepithelial layer of a mammalian umbilical cord tissue in direct contact with a growth substrate; and culturing the subepithelial layer such that the isolated cell from the subepithelial layer is capable of self-renewal and culture expansion, wherein the isolated cell expresses at least three cell markers selected from the group consisting of CD29, CD73, CD90, CD166, SSEA4, CD9, CD44, CD146, or CD105, and wherein the isolated cell does not express NANOG and at least five cell markers selected from the group consisting of CD45, CD34, CD14, CD79, CD106, CD86, CD80, CD19, CD117, Stro-1, or HLA-DR.

Preferred methods include embodiments wherein said cells expresses CD29, CD73, CD90, CD166, SSEA4, CD9, CD44, CD146, and CD105.

Preferred methods include embodiments wherein said cells express CD105.

Preferred methods include embodiments wherein said cells express CD146.

Preferred methods include embodiments wherein said cells express CD44.

Preferred methods include embodiments wherein said cells express CD9.

Preferred methods include embodiments wherein said cells express SSEA4.

Preferred methods include embodiments wherein said cells express CD166.

Preferred methods include embodiments wherein said cells express CD90.

Preferred methods include embodiments wherein said cells expresses CD73.

Preferred methods include embodiments wherein said cells expresses CD29.

Preferred methods include embodiments wherein said cells do not express CD45, CD34, CD14, CD79, CD106, CD86, CD80, CD19, CD117, Stro-1, and HLA-DR.

Preferred methods include embodiments wherein said cells are positive for SOX2.

Preferred methods include embodiments wherein said cells are positive for OCT4.

Preferred methods include embodiments wherein said cells are positive for SOX2 and OCT4.

Preferred methods include embodiments wherein said cells are capable of differentiation into a cell type selected from the group consisting of adipocytes, chondrocytes, osteocytes, cardiomyocytes, endothelial cells, and myocytes.

Preferred methods include embodiments wherein said regenerative cell is activated in vitro in order to augment its ability to produce factors that induce an M2/myeloid derived suppressor cell phenotype in said adherent monocytes.

Preferred methods include embodiments wherein said regenerative cells is activated by addition of lymphocyte conditioned media.

Preferred methods include embodiments wherein said conditioned media is generated by culture of allogeneic T cells with a population of cells that contains an allogeneic HLA.

Preferred methods include embodiments wherein said conditioned media is removed after a 24 hour to 2 week culture.

Preferred methods include embodiments wherein said conditioned media is removed after a 48 hour to 1 week culture.

Preferred methods include embodiments wherein said conditioned media is removed after a 72 hour culture.

Preferred methods include embodiments wherein said conditioned media is added to said adherent cells of claim 1 are cultured with said conditioned media for a period of 30 minutes to 3 months.

Preferred methods include embodiments wherein said conditioned media is added to said adherent cells of claim 1 are cultured with said conditioned media for a period of 12 hours to 1 months.

Preferred methods include embodiments wherein said conditioned media is added to said adherent cells of claim 1 are cultured with said conditioned media for a period of 7 days to 14 days.

Preferred methods include embodiments wherein said culture is performed in the presence of an mTOR inhibitor.

Preferred methods include embodiments wherein said mTOR inhibitor is rapamycin.

Preferred methods include embodiments wherein said mTOR inhibitor is temsirolimus.

Preferred methods include embodiments wherein said mTOR inhibitor is everolimus.

Preferred methods include embodiments wherein said mTOR inhibitor is deforolimus.

Preferred methods include embodiments wherein said mTOR inhibitor is umirolimus.

Preferred methods include embodiments wherein said mTOR inhibitor is Zotarolimus.

Preferred methods include embodiments, wherein culture of adherent monocytic lineage cells is performed with culture of regenerative cell supernatant in the presence of an inhibitor of NF-kappa B activity.

Preferred methods include embodiments wherein said suppression of NF-kappa B activity is achieved by administration of an antisense molecule targeting NF-kappa B or molecules in the NF-kappa B pathway.

Preferred methods include embodiments wherein said suppression of NF-kappa B activity is achieved by administration of a molecule capable of triggering RNA interference targeting NF-kappa B or molecules in the NF-kappa B pathway.

Preferred methods include embodiments wherein said suppression of NF-kappa B activity is achieved by gene editing means targeting NF-kappa B or molecules in the NF-kappa B pathway.

Preferred methods include embodiments wherein said suppression of NF-kappa B activity is achieved by administration of decoy oligonucleotides capable of blocking NF-kappa B or molecules in the NF-kappa B pathway.

Preferred methods include embodiments wherein said suppression of NF-kappa B activity is achieved by administration of a small molecule blocker of NF-kappa B activity.

Preferred methods include embodiments wherein said small molecule blocker of NF-kappa B activity is selected from a group comprising of: Calagualine (fern derivative), Conophylline (Ervatamia microphylla), Evodiamine (Evodiae fructus component), Geldanamycin, Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic).

Preferred methods include embodiments wherein culture of adherent monocytic lineage cells is performed with culture of regenerative cell supernatant in whole or in part in the presence of hypoxic preconditioning.

Preferred methods include embodiments wherein said hypoxic preconditioning comprises exposure of said cells to conditions allowing for upregulation of HIF-1 alpha gene expression by 25% or more as compared to baseline culture.

Preferred methods include embodiments wherein said hypoxic preconditioning comprises exposure of said cells to conditions allowing for upregulation of HIF-1 alpha gene expression by 50% or more as compared to baseline culture.

Preferred methods include embodiments wherein said hypoxic preconditioning comprises exposure of said cells to conditions allowing for nuclear translocation of HIF-1 alpha protein by 25% or more as compared to baseline culture.

Preferred methods include embodiments wherein said hypoxic preconditioning comprises exposure of said cells to conditions allowing for nuclear translocation of HIF-1 alpha protein by 50% or more as compared to baseline culture.

Preferred methods include embodiments wherein hypoxic preconditioning involves culturing of said cells with agents capable of imitating hypoxia.

Preferred methods include embodiments wherein said hypoxic preconditioning involves culture of cells with carbon monoxide.

Preferred methods include embodiments wherein said culture with carbon monoxide is exposing cells to a gas composition comprising: a. 0% to about 79% by weight nitrogen gas; b. about 21% to about 100% by weight oxygen gas; and c. about 0.0000001% to less than 0.3% by weight carbon monoxide gas.

Preferred methods include embodiments wherein said composition contains 0% nitrogen and about 100% oxygen.

Preferred methods include embodiments wherein said composition contains 0% nitrogen and the amount of carbon monoxide in said composition ranges from about 0.0001% to about 0.075%.

Preferred methods include embodiments wherein the amount of carbon monoxide in said composition ranges from about 0.0001% to about 0.075%.

Preferred methods include embodiments wherein said composition contains 0% nitrogen and the amount of carbon monoxide in said composition ranges from about 0.005% to about 0.05%.

Preferred methods include embodiments wherein the amount of carbon monoxide in said composition ranges from about 0.005% to about 0.05%.

Preferred methods include embodiments wherein said hypoxic preconditioning is performed by culturing monocytic cells or progeny thereof in conditions of 1% to 15% of oxygen in terms of volume ratio.

Preferred methods include embodiments wherein said cells are cultured for at least one passage.

Preferred methods include embodiments wherein monocytes and/or monocytic cells are modified for enhanced survival and/or activity.

Preferred methods include embodiments wherein said enhanced survival and/or activity is accomplished by culturing said monocytes and/or monocytic cells with an epigenetic modulator.

Preferred methods include embodiments wherein said epigenetic modulator comprises a histone deacetylase inhibitor.

Preferred methods include embodiments wherein said histone deacetylase inhibitor is valproic acid.

Preferred methods include embodiments wherein said histone deacetylase inhibitor is vorinostat.

Preferred methods include embodiments wherein said histone deacetylase inhibitor is entinostat.

Preferred methods include embodiments wherein said histone deacetylase inhibitor is panobinostat.

Preferred methods include embodiments wherein said histone deacetylase inhibitor is trichostatin A.

Preferred methods include embodiments wherein said histone deacetylase inhibitor is mocetinostat.

Preferred methods include embodiments wherein said histone deacetylase inhibitor is belinostat.

Preferred methods include embodiments wherein said histone deacetylase inhibitor is FK228.

Preferred methods include embodiments wherein said histone deacetylase inhibitor is MC1568.

Preferred methods include embodiments wherein said histone deacetylase inhibitor is tubastatin.

Preferred methods include embodiments wherein said histone deacetylase inhibitor is sodium butyrate.

Preferred methods include embodiments wherein said histone deacetylase inhibitor is sulforaphane.

Preferred methods include embodiments wherein said epigenetic modulator is a DNA methyltransferase inhibitor.

Preferred methods include embodiments wherein said DNA methyltransferase inhibitor is 5 azacytidine.

Preferred methods include embodiments wherein said monocytes and/or monocytic cells are primed by culture with a GSK-3 inhibitor.

Preferred methods include embodiments wherein said GSK-3 inhibitor is lithium or a lithium salt.

Preferred embodiments include methods of treating an inflammatory condition comprising: a) obtaining a monocytic population; b) culturing said monocytic cell population in the presence of a regenerative cell; c) providing conditions such that said regenerative cell endows said monocytic cell population with enhanced regenerative and/or immune modulatory potential; and d) administering said monocytic cell population.

Preferred methods include embodiments wherein said monocytic cell population is a monocyte.

Preferred methods include embodiments wherein said monocyte is plastic adherent.

Preferred methods include embodiments wherein said monocyte expresses CD2.

Preferred methods include embodiments wherein said monocyte expresses CD1 lb.

Preferred methods include embodiments wherein said monocyte expresses CD11d.

Preferred methods include embodiments wherein said monocyte expresses CD14.

Preferred methods include embodiments wherein said monocyte expresses CD16.

Preferred methods include embodiments wherein said monocyte expresses CD31.

Preferred methods include embodiments wherein said monocyte expresses CD56.

Preferred methods include embodiments wherein said monocyte expresses CD62L.

Preferred methods include embodiments wherein said monocyte expresses CD192.

Preferred methods include embodiments wherein said monocyte expresses serotonin receptor.

Preferred methods include embodiments wherein said monocyte expresses CX3CR1.

Preferred methods include embodiments wherein said monocyte expresses CD115.

Preferred methods include embodiments wherein said monocyte expresses CD22.

Preferred methods include embodiments wherein said monocyte expresses CD29.

Preferred methods include embodiments wherein said monocyte expresses CD105.

Preferred methods include embodiments wherein said monocyte expresses CXCR3.

Preferred methods include embodiments wherein said monocyte expresses CXCR4.

Preferred methods include embodiments wherein said monocyte expresses interleukin 1 receptor.

Preferred methods include embodiments wherein said monocyte expresses interleukin 4 receptor.

Preferred methods include embodiments wherein said monocyte expresses interleukin 6 receptor.

Preferred methods include embodiments wherein said monocyte expresses interleukin 8 receptor.

Preferred methods include embodiments wherein said monocyte expresses interleukin 10 receptor.

Preferred methods include embodiments wherein said monocyte expresses interleukin 11 receptor.

Preferred methods include embodiments wherein said monocyte expresses interleukin 12 receptor.

Preferred methods include embodiments wherein said monocyte expresses interleukin 13 receptor.

Preferred methods include embodiments wherein said monocyte expresses interleukin 15 receptor.

Preferred methods include embodiments wherein said monocyte expresses interleukin 16 receptor.

Preferred methods include embodiments wherein said monocyte expresses interleukin 17 receptor.

Preferred methods include embodiments wherein said monocyte expresses interleukin 18 receptor.

Preferred methods include embodiments wherein said monocyte expresses interleukin 19 receptor.

Preferred methods include embodiments wherein said monocyte expresses interleukin 20 receptor.

Preferred methods include embodiments wherein said monocyte expresses interleukin 21 receptor.

Preferred methods include embodiments wherein said monocyte expresses interleukin 22 receptor.

Preferred methods include embodiments wherein said monocyte expresses interleukin 23 receptor.

Preferred methods include embodiments wherein said monocyte expresses interleukin 26 receptor.

Preferred methods include embodiments wherein said monocyte expresses interleukin 27 receptor.

Preferred methods include embodiments wherein said monocyte expresses interleukin 33 receptor.

Preferred methods include embodiments wherein said monocyte expresses interleukin 35 receptor.

Preferred methods include embodiments wherein said monocyte expresses TNF-alpha receptor.

Preferred methods include embodiments wherein said monocyte expresses TNF-beta receptor.

Preferred methods include embodiments wherein said monocyte expresses interferon alpha receptor.

Preferred methods include embodiments wherein said monocyte expresses interferon beta receptor.

Preferred methods include embodiments wherein said monocyte expresses interferon gamma receptor.

Preferred methods include embodiments wherein said monocyte expresses interferon omega receptor.

Preferred methods include embodiments wherein said monocyte expresses interleukin 21 receptor.

Preferred methods include embodiments wherein said monocytes are derived from monocytic progenitors.

Preferred methods include embodiments wherein said monocytic progenitors are derived from umbilical cord blood.

Preferred methods include embodiments wherein said monocytic progenitors are derived from umbilical cord tissue.

Preferred methods include embodiments wherein said monocytic progenitors are derived from umbilical cord Wharton's Jelly.

Preferred methods include embodiments wherein said monocytic progenitors are derived from placenta.

Preferred methods include embodiments wherein said monocytic progenitors are derived from bone marrow.

Preferred methods include embodiments wherein said monocytic progenitors are derived from peripheral blood.

Preferred methods include embodiments wherein said monocytic progenitors are derived from a pluripotent stem cell.

Preferred methods include embodiments wherein said pluripotent stem cell is an embryonic stem cell.

Preferred methods include embodiments wherein said pluripotent stem cell is an inducible pluripotent stem cell.

Preferred methods include embodiments wherein said pluripotent stem cell is a very small embryonic-like stem cell.

Preferred methods include embodiments wherein said very small embryonic-like stem cells express CD11c.

Preferred methods include embodiments wherein said very small embryonic-like stem cells express STRO-1.

Preferred methods include embodiments wherein said very small embryonic-like stem cells express SSEA4.

Preferred methods include embodiments wherein said very small embryonic-like stem cells express TRA-1.

Preferred methods include embodiments wherein said very small embryonic-like stem cells express c-met.

Preferred methods include embodiments wherein said very small embryonic-like stem cells express OCT-4.

Preferred methods include embodiments wherein said very small embryonic-like stem cells express Nanog.

Preferred methods include embodiments wherein said very small embryonic-like stem cells express interleukin 3 receptor.

Preferred methods include embodiments wherein said very small embryonic-like stem cells express vegf receptor-1.

Preferred methods include embodiments wherein said very small embryonic-like stem cells express c-kit

Preferred methods include embodiments wherein said very small embryonic-like stem cells express interleukin 6 receptor.

Preferred methods include embodiments wherein said very small embryonic-like stem cells express interleukin 8 receptor.

Preferred methods include embodiments wherein said very small embryonic-like stem cells express M-CSF receptor.

Preferred methods include embodiments wherein said very small embryonic-like stem cells express G-CSF receptor.

Preferred methods include embodiments wherein said very small embryonic-like stem cells express GM-CSF receptor.

Preferred methods include embodiments wherein said very small embryonic-like stem cells express interleukin 11 receptor.

Preferred methods include embodiments wherein said very small embryonic-like stem cells express interleukin 12 receptor.

Preferred methods include embodiments wherein said very small embryonic-like stem cells express Hepatocyte Growth Factor receptor.

Preferred methods include embodiments wherein said very small embryonic-like stem cells express TNF-alpha receptor p55.

Preferred methods include embodiments wherein said very small embryonic-like stem cells express TNF-alpha receptor p75.

Preferred methods include embodiments wherein said very small embryonic-like stem cells express FGF-1 receptor.

Preferred methods include embodiments wherein said very small embryonic-like stem cells express FGF-2 receptor.

Preferred methods include embodiments wherein said very small embryonic-like stem cells express IGF-1 receptor.

Preferred methods include embodiments wherein said very small embryonic-like stem cells express EGF receptor.

Preferred methods include embodiments wherein said very small embryonic-like stem cells express PDGF-BB receptor.

Preferred methods include embodiments wherein said very small embryonic-like stem cells express angiopoietin receptor.

Preferred methods include embodiments wherein said very small embryonic-like stem cells express TGF-beta receptor.

Preferred methods include embodiments wherein said very small embryonic-like stem cells express soluble TNF alpha receptor.

Preferred methods include embodiments wherein said very small embryonic-like stem cells express fas ligand.

Preferred methods include embodiments wherein said peripheral blood used to generate monocytes is extracted from a patient in which monocytic progenitors have been mobilized.

Preferred methods include embodiments wherein said peripheral blood that has been mobilized was mobilized by treatment of the donor with G-CSF.

Preferred methods include embodiments wherein said peripheral blood that has been mobilized was mobilized by treatment of the donor with M-CSF.

Preferred methods include embodiments wherein said peripheral blood that has been mobilized was mobilized by treatment of the donor with GM-CSF.

Preferred methods include embodiments wherein said peripheral blood that has been mobilized was mobilized by treatment of the donor with IL-3.

Preferred methods include embodiments wherein said peripheral blood that has been mobilized was mobilized by treatment of the donor with a CXCR-4 antagonist.

Preferred methods include embodiments wherein said CXCR-4 antagonist is mozibil.

Preferred methods include embodiments wherein said monocytes are generated from hematopoietic stem cells.

Preferred methods include embodiments wherein said hematopoietic stem cells are derived from bone marrow.

Preferred methods include embodiments wherein said hematopoietic stem cells are derived from umbilical cord tissue.

Preferred methods include embodiments wherein said hematopoietic stem cells are derived from umbilical cord Wharton's Jelly.

Preferred methods include embodiments wherein said hematopoietic stem cells are derived from umbilical cord blood.

Preferred methods include embodiments wherein said hematopoietic stem cells are derived from peripheral blood.

Preferred methods include embodiments wherein said hematopoietic stem cells are derived from mobilized peripheral blood.

Preferred methods include embodiments wherein said peripheral blood that has been mobilized was mobilized by treatment of the donor with G-CSF.

Preferred methods include embodiments wherein said peripheral blood that has been mobilized was mobilized by treatment of the donor with M-CSF.

Preferred methods include embodiments wherein said peripheral blood that has been mobilized was mobilized by treatment of the donor with GM-CSF.

Preferred methods include embodiments wherein said peripheral blood that has been mobilized was mobilized by treatment of the donor with IL-3.

Preferred methods include embodiments wherein said peripheral blood that has been mobilized was mobilized by treatment of the donor with a CXCR-4 antagonist.

Preferred methods include embodiments wherein said CXCR-4 antagonist is mozibil.

Preferred methods include embodiments wherein said hematopoietic stem cells proliferate in response to interleukin-3 stimulation.

Preferred methods include embodiments wherein said hematopoietic stem cells proliferate more than 50% as compared to resting conditions when cultured with 1-1000 pg/ml of interleukin-3.

Preferred methods include embodiments wherein said hematopoietic stem cells proliferate in response to interleukin-6 stimulation.

Preferred methods include embodiments wherein said hematopoietic stem cells proliferate more than 50% as compared to resting conditions when cultured with 1-1000 pg/ml of interleukin-6.

Preferred methods include embodiments wherein said hematopoietic stem cells proliferate in response to interleukin-11 stimulation.

Preferred methods include embodiments wherein said hematopoietic stem cells proliferate more than 50% as compared to resting conditions when cultured with 1-1000 pg/ml of interleukin-11.

Preferred methods include embodiments wherein said hematopoietic stem cells proliferate in response to interleukin-20 stimulation.

Preferred methods include embodiments wherein said hematopoietic stem cells proliferate more than 50% as compared to resting conditions when cultured with 1-1000 pg/ml of interleukin-20.

Preferred methods include embodiments wherein said hematopoietic stem cells proliferate in response to M-CSF stimulation.

Preferred methods include embodiments wherein said hematopoietic stem cells proliferate more than 50% as compared to resting conditions when cultured with 1-1000 pg/ml of M-CSF.

Preferred methods include embodiments wherein said hematopoietic stem cells proliferate in response to G-CSF stimulation.

Preferred methods include embodiments wherein said hematopoietic stem cells proliferate more than 50% as compared to resting conditions when cultured with 1-1000 pg/ml of G-CSF.

Preferred methods include embodiments wherein said hematopoietic stem cells proliferate in response to HGF-1 stimulation.

Preferred methods include embodiments wherein said hematopoietic stem cells proliferate more than 50% as compared to resting conditions when cultured with 1-1000 pg/ml of HGF-1.

Preferred methods include embodiments wherein said hematopoietic stem cells proliferate in response to GM-CSF stimulation.

Preferred methods include embodiments wherein said hematopoietic stem cells proliferate more than 50% as compared to resting conditions when cultured with 1-1000 pg/ml of GM-CSF.

Preferred methods include embodiments wherein said hematopoietic stem cells proliferate in response to IGF-1 stimulation.

Preferred methods include embodiments wherein said hematopoietic stem cells proliferate more than 50% as compared to resting conditions when cultured with 1-1000 pg/ml of IGF-1.

Preferred methods include embodiments wherein said hematopoietic stem cells proliferate in response to stem cell factor stimulation.

Preferred methods include embodiments wherein said hematopoietic stem cells proliferate more than 50% as compared to resting conditions when cultured with 1-1000 pg/ml of stem cell factor.

Preferred methods include embodiments wherein said hematopoietic stem cells proliferate in response to PDGF-BB stimulation.

Preferred methods include embodiments wherein said hematopoietic stem cells proliferate more than 50% as compared to resting conditions when cultured with 1-1000 pg/ml of PDGF-BB.

Preferred methods include embodiments wherein said hematopoietic stem cell expresses 2B4.

Preferred methods include embodiments wherein said hematopoietic stem cell expresses ABCG2.

Preferred methods include embodiments wherein said hematopoietic stem cell expresses aldehyde dehydrogenase.

Preferred methods include embodiments wherein said hematopoietic stem cell expresses BMI-1.

Preferred methods include embodiments wherein said hematopoietic stem cell expresses CD93.

Preferred methods include embodiments wherein said hematopoietic stem cell expresses CD34.

Preferred methods include embodiments wherein said hematopoietic stem cell expresses CD38.

Preferred methods include embodiments wherein said hematopoietic stem cell expresses CD44.

Preferred methods include embodiments wherein said hematopoietic stem cell expresses CD45.

Preferred methods include embodiments wherein said hematopoietic stem cell expresses CD48.

Preferred methods include embodiments wherein said hematopoietic stem cell expresses CD90.

Preferred methods include embodiments wherein said hematopoietic stem cell expresses CD117.

Preferred methods include embodiments wherein said hematopoietic stem cell expresses CD133

Preferred methods include embodiments wherein said hematopoietic stem cell expresses CDCP1.

Preferred methods include embodiments wherein said hematopoietic stem cell expresses CXCR4.

Preferred methods include embodiments wherein said hematopoietic stem cell expresses CD105.

Preferred methods include embodiments wherein said hematopoietic stem cell expresses EPCR.

Preferred methods include embodiments wherein said hematopoietic stem cell expresses EPO receptor.

Preferred methods include embodiments wherein said hematopoietic stem cell expresses ESAM.

Preferred methods include embodiments wherein said hematopoietic stem cell expresses EVI-1.

Preferred methods include embodiments wherein said hematopoietic stem cell expresses Flt-3.

Preferred methods include embodiments wherein said hematopoietic stem cell expresses PD-1.

Preferred methods include embodiments wherein said hematopoietic stem cell expresses PD-Ll.

Preferred methods include embodiments wherein said hematopoietic stem cell expresses VISTA.

Preferred methods include embodiments wherein said hematopoietic stem cell expresses GATA-2.

Preferred methods include embodiments wherein said hematopoietic stem cell expresses GFI-1.

Preferred methods include embodiments wherein said hematopoietic stem cell expresses hematopoietic lineage marker.

Preferred methods include embodiments wherein said hematopoietic stem cell expresses CD49f.

Preferred methods include embodiments wherein said hematopoietic stem cell expresses Mcl-1.

Preferred methods include embodiments wherein said hematopoietic stem cell expresses MYB.

Preferred methods include embodiments wherein said hematopoietic stem cell expresses PLZF.

Preferred methods include embodiments wherein said hematopoietic stem cell expresses podocalyxin.

Preferred methods include embodiments wherein said hematopoietic stem cell expresses prominin-2.

Preferred methods include embodiments wherein said hematopoietic stem cell expresses PTEN.

Preferred methods include embodiments, wherein said hematopoietic stem cell expresses PU-1.

Preferred methods include embodiments wherein said hematopoietic stem cell expresses Sca-1.

Preferred methods include embodiments wherein said hematopoietic stem cell expresses CD150.

Preferred methods include embodiments wherein said hematopoietic stem cell expresses Spi-B.

Preferred methods include embodiments wherein said hematopoietic stem cell expresses STATS a/b.

Preferred methods include embodiments wherein said hematopoietic stem cell expresses STAT 5a.

Preferred methods include embodiments wherein said hematopoietic stem cell expresses STAT 5b.

Preferred methods include embodiments wherein said hematopoietic stem cell expresses CD106.

Preferred methods include embodiments wherein said hematopoietic stem cell expresses VEGFR2.

Preferred methods include embodiments wherein said monocytic cell is cultured in the presence of said regenerative cell conditioned media.

Preferred methods include embodiments wherein said monocytic cell is cultured in the presence of said regenerative cell derived exosomes.

Preferred methods include embodiments wherein said regenerative cell exosome is purified based on size.

Preferred methods include embodiments wherein said regenerative cell exosome is purified based on weight.

Preferred methods include embodiments wherein said regenerative cell exosome is purified based on affinity towards a substrate.

Preferred methods include embodiments wherein said regenerative cell exosome is purified based on affinity towards a lectin.

Preferred methods include embodiments wherein said lectin is Galanthus nivalis lectin.

Preferred methods include embodiments wherein said lectin is concanavalin A.

Preferred methods include embodiments wherein said lectin is phytohemagluttinin.

Preferred methods include embodiments wherein said lectin is mannose binding lectin.

Preferred methods include embodiments wherein said lectin is Griffonia Bandeiraea simplicifolia lectin-I.

Preferred methods include embodiments wherein said lectin is Lotus tetragonolobus lectin.

Preferred methods include embodiments wherein said lectin is Aspergillus oryzae lectin.

Preferred methods include embodiments wherein said lectin is icinus communis agglutinin.

Preferred methods include embodiments wherein said lectin is Artocarpus integer galactose binding lectin.

Preferred methods include embodiments wherein said lectin is Artocarpus integer mannose binding lectin.

Preferred methods include embodiments wherein said lectin is Narcissus pseudonarcissus lectin.

Preferred methods include embodiments wherein said lectin is Sambucus nigra agglutinin.

Preferred methods include embodiments wherein said lectin is Artocarpus heterophyllus lectin.

Preferred methods include embodiments wherein said lectin is Lens culinaris hemagglutinin.

Preferred methods include embodiments wherein said lectin is Maackia amurensis lectin II.

Preferred methods include embodiments wherein said lectin is Aleuria aurantia lectin.

Preferred methods include embodiments wherein said lectin is (Arachis hypogaea) agglutinin.

Preferred methods include embodiments wherein said lectin is (Pinellia pedatisecta) agglutinin.

Preferred methods include embodiments wherein said lectin is (Phytolacca americana) mitogen lectin.

Preferred methods include embodiments wherein said lectin is (Phaseolus vulgaris) lectin.

Preferred methods include embodiments wherein said lectin is (Datura stramonium) lectin.

Preferred methods include embodiments wherein said lectin is (Triticum vulgaris) agglutinin.

Preferred methods include embodiments wherein said lectin is (Agaricus bisporus) lectin.

Preferred methods include embodiments wherein said regenerative cell derived exosome expresses miR-146a.

Preferred methods include embodiments wherein said regenerative cell derived exosome expresses miR22.

Preferred methods include embodiments wherein said regenerative cell derived exosome expresses miR24.

Preferred methods include embodiments wherein said regenerative cell derived exosome expresses miR210.

Preferred methods include embodiments wherein said regenerative cell derived exosome expresses miR150.

Preferred methods include embodiments wherein said regenerative cell derived exosome expresses miR-140-3p.

Preferred methods include embodiments wherein said regenerative cell derived exosome expresses miR-19a.

Preferred methods include embodiments wherein said regenerative cell derived exosome expresses miR-27b.

Preferred methods include embodiments wherein said regenerative cell derived exosome expresses miR-19b.

Preferred methods include embodiments wherein said regenerative cell derived exosome expresses miR-27a.

Preferred methods include embodiments wherein said regenerative cell derived exosome expresses miR-376c.

Preferred methods include embodiments wherein said regenerative cell derived exosome expresses miR-128.

Preferred methods include embodiments wherein said regenerative cell derived exosome expresses miR-320a.

Preferred methods include embodiments wherein said regenerative cell derived exosome expresses miR-143.

Preferred methods include embodiments wherein said regenerative cell derived exosome expresses miR-21.

Preferred methods include embodiments wherein said regenerative cell derived exosome expresses miR-130a.

Preferred methods include embodiments wherein said regenerative cell derived exosome expresses miR-9.

Preferred methods include embodiments wherein said regenerative cell derived exosome expresses miR-185.

Preferred methods include embodiments wherein said regenerative cell derived exosome expresses miR-23a.

Preferred methods include embodiments wherein said regenerative cell derived exosome express one or more markers selected from the group comprising of miR-146a, miR-22, or miR-24.

Preferred methods include embodiments wherein said regenerative cell derived exosomes express CD63.

Preferred methods include embodiments wherein said regenerative cell derived exosomes express CD81.

Preferred methods include embodiments wherein said regenerative cell derived exosomes express CD81 and CD63.

Preferred methods include embodiments wherein said regenerative cell derived exosomes express HLA-G.

Preferred methods include embodiments wherein said regenerative cell derived exosomes express latency associated protein.

Preferred methods include embodiments wherein said regenerative cell derived exosomes express TNF receptor p55.

Preferred methods include embodiments wherein said regenerative cell derived exosomes express TNF receptor p75.

Preferred methods include embodiments wherein said regenerative cell derived exosomes express ILT-3.

Preferred methods include embodiments wherein said regenerative cell derived exosomes express ILT-4.

Preferred methods include embodiments wherein said regenerative cell derived exosomes express galectin 3.

Preferred methods include embodiments wherein said regenerative cell derived exosomes express galectin 4.

Preferred methods include embodiments wherein said regenerative cell derived exosomes express galectin 6.

Preferred methods include embodiments wherein said regenerative cell derived exosomes express galectin 9.

Preferred methods include embodiments wherein said regenerative cell derived exosomes express TRAIL.

Preferred methods include embodiments wherein said regenerative cell derived exosomes express Fas ligand.

Preferred methods include embodiments wherein said regenerative cell derived exosomes express MHC 1.

Preferred methods include embodiments wherein said regenerative cell derived exosomes express interleukin 1 receptor.

Preferred methods include embodiments wherein said regenerative cell derived exosomes express interleukin 2 receptor.

Preferred methods include embodiments wherein said regenerative cell derived exosomes express interleukin 4 receptor.

Preferred methods include embodiments wherein said regenerative cell derived exosomes express interleukin 6 receptor.

Preferred methods include embodiments wherein said regenerative cell derived exosomes express interleukin 8 receptor.

Preferred methods include embodiments wherein said regenerative cell derived exosomes express interleukin 10 receptor.

Preferred methods include embodiments wherein said regenerative cell derived exosomes express interleukin 12 receptor.

Preferred methods include embodiments wherein said regenerative cell derived exosomes express interleukin 15 receptor.

Preferred methods include embodiments wherein said regenerative cell derived exosomes express interleukin 17 receptor.

Preferred methods include embodiments wherein said regenerative cell derived exosomes express interleukin 13 receptor.

Preferred methods include embodiments wherein said regenerative cell derived exosomes express interleukin 18 receptor.

Preferred methods include embodiments wherein said regenerative cell derived exosomes are comprised of a lipid bilayer.

Preferred methods include embodiments wherein said regenerative cell derived exosomes are of endosomal origin.

Preferred methods include embodiments wherein said regenerative cell derived exosomes range in size from 19-300 nm.

Preferred methods include embodiments wherein said regenerative cell derived exosomes range in size from 35-200 nm.

Preferred methods include embodiments wherein said regenerative cell derived exosomes range in size from 50-100 nm.

Preferred methods include embodiments wherein said regenerative cell derived exosomes range in size from 60-90 nm.

Preferred methods include embodiments wherein said regenerative cell derived exosomes range in size from 65-90 nm.

Preferred methods include embodiments wherein said regenerative cell derived exosomes range in size from 70-90 nm.

Preferred methods include embodiments wherein said regenerative cell derived exosomes possess a cup-shaped morphology as revealed by electron microscopy.

Preferred methods include embodiments wherein said regenerative cell derived exosomes are concentrated from conditioned media of regenerative cells by differential ultracentrifugation, to separate the exosomes from the supernatants of cultured cells.

Preferred methods include embodiments wherein ultracentrifugation allows for separation of exosomes from nonmembranous particles by exploiting their relatively low buoyant density.

Preferred methods include embodiments wherein a size exclusion means is further utilized to purify exosomes.

Preferred methods include embodiments wherein said size exclusion means comprises of filtration means.

Preferred methods include embodiments wherein said size filtration means allows for selection of exosomes possessing a diameter ranging from 30-200 nm.

Preferred methods include embodiments wherein said size filtration means allows for selection of exosomes possessing a diameter ranging from 40-100 nm.

Preferred methods include embodiments wherein said size filtration means allows for selection of exosomes possessing a diameter ranging from 40-50 nm.

Preferred methods include embodiments wherein said size filtration means allows for selection of exosomes possessing a diameter ranging from 50-60 nm.

Preferred methods include embodiments wherein said size filtration means allows for selection of exosomes possessing a diameter ranging from 60-70 nm.

Preferred methods include embodiments wherein said size filtration means allows for selection of exosomes possessing a diameter ranging from 60-70 nm.

Preferred methods include embodiments wherein said regenerative cell is cell capable of renewing itself while having ability to generate cells of other lineages.

Preferred methods include embodiments wherein said cell lineage is endoderm.

Preferred methods include embodiments wherein said cell lineage is ectoderm.

Preferred methods include embodiments wherein said cell lineage is mesoderm.

Preferred methods include embodiments wherein said regenerative cells are stem cells.

Preferred methods include embodiments wherein said stem cells are selected from a group comprising of a) embryonic stem cells; b) hematopoietic stem cells; c) mesenchymal stem cells; d) very small embryonic like stem cells; e) inducible pluripotent stem cells; f) bone marrow stem cells; g) amniotic fluid stem cells; h) neuronal stem cells; i) parthenogenically derived stem cells; j) cord blood stem cells; k) placental stem cells; 1) bone marrow stem cells; m) germinal stem cells; n) hair follicle stem cells; o) adipose derived stem cells; p) reprogrammed stem cells; q) peripheral blood derived stem cells; r) peripheral blood mesenchymal stem cells; s) endometrial regenerative cells; t) fallopian tube derived stem cells; u) dermal stem cells; v) side population stem cells; and w) subepithelial umbilical cord tissue stem cells.

586. The method of claim 585, wherein said embryonic stem cells are totipotent.

587. The method of claim 585, wherein said embryonic stem cells express stage-specific embryonic antigens (SSEA) 3.

Preferred methods include embodiments wherein said embryonic stem cells express stage-specific embryonic antigens (SSEA) 4.

Preferred methods include embodiments wherein said embryonic stem cells express Tra-1-60.

Preferred methods include embodiments wherein said embryonic stem cells express Tra-1-81.

Preferred methods include embodiments wherein said embryonic stem cells express Oct-3.

Preferred methods include embodiments wherein said embryonic stem cells express Oct-4.

Preferred methods include embodiments wherein said embryonic stem cells express SOX-2.

Preferred methods include embodiments wherein said embryonic stem cells express IL-17 receptor.

Preferred methods include embodiments wherein said cord blood stem cells are multipotent and capable of differentiating into hematopoietic and mesenchymal lineages.

Preferred methods include embodiments wherein cord blood stem cells are capable of differentiating along the mesenchymal lineage.

Preferred methods include embodiments wherein said cord blood hematopoietic stem cell expresses CD34.

Preferred methods include embodiments wherein said cord blood hematopoietic stem cell expresses CD133.

Preferred methods include embodiments wherein said cord blood hematopoietic stem cell expresses flk-2.

Preferred methods include embodiments wherein said cord blood hematopoietic stem cell expresses interleukin-3 receptor.

Preferred methods include embodiments wherein said cord blood hematopoietic stem cell expresses c-met.

Preferred methods include embodiments wherein said cord blood hematopoietic stem cell expresses interleukin 6 receptor.

Preferred methods include embodiments wherein said cord blood hematopoietic stem cell expresses interleukin 8 receptor.

Preferred methods include embodiments wherein said cord blood hematopoietic stem cell expresses interleukin-11 receptor.

Preferred methods include embodiments wherein said cord blood hematopoietic stem cell expresses follistatin receptor.

Preferred methods include embodiments wherein said cord blood hematopoietic stem cell expresses TPO receptor.

Preferred methods include embodiments wherein said cord blood hematopoietic stem cell expresses BMP-2 receptor.

Preferred methods include embodiments herein said cord blood mesenchymal stem cell expresses CD90.

Preferred methods include embodiments herein said cord blood mesenchymal stem cell expresses CD105.

Preferred methods include embodiments herein said cord blood mesenchymal stem cell expresses Her2.

Preferred methods include embodiments herein said cord blood mesenchymal stem cell expresses FGF-1 receptor.

Preferred methods include embodiments herein said cord blood mesenchymal stem cell expresses FGF-2 receptor.

Preferred methods include embodiments herein said cord blood mesenchymal stem cell expresses EGF-1 receptor.

Preferred methods include embodiments herein said cord blood mesenchymal stem cell expresses IGF-1 receptor.

Preferred methods include embodiments herein said cord blood mesenchymal stem cell expresses c-met.

Preferred methods include embodiments herein said cord blood mesenchymal stem cell expresses Mg-SOD at approximately 2-fold higher than a fibroblast cell.

Preferred methods include embodiments wherein said cord blood stem cells express SSEA-3.

Preferred methods include embodiments wherein said cord blood stem cells express SSEA-4.

Preferred methods include embodiments wherein said cord blood stem cells express CD9.

Preferred methods include embodiments wherein said cord blood stem cells express CD34.

Preferred methods include embodiments wherein said cord blood stem cells express CD-133.

Preferred methods include embodiments wherein said cord blood stem cells express c-kit.

Preferred methods include embodiments wherein said cord blood stem cells express OCT-4.

Preferred methods include embodiments wherein said cord blood stem cells express NANOG.

Preferred methods include embodiments wherein said cord blood stem cells express CXCR-4.

Preferred methods include embodiments wherein said cord blood stem cells express c-met.

Preferred methods include embodiments wherein said cord blood stem cells express CD-56.

Preferred methods include embodiments wherein said cord blood stem cells express CD-57.

Preferred methods include embodiments wherein said cord blood stem cells express FoxP3.

Preferred methods include embodiments wherein said cord blood stem cells do not CD-3.

Preferred methods include embodiments wherein said cord blood stem cells do not CD-14.

Preferred methods include embodiments wherein said cord blood stem cells do not CD-16.

Preferred methods include embodiments wherein said cord blood stem cells do not CD-11b.

Preferred methods include embodiments wherein said placental stem cells are isolated from the placental structure.

Preferred methods include embodiments wherein said placental stem cells express one or more antigens selected from a group comprising: a) Oct-4; b) Rex-1; c) CD9; d) CD13; e) CD29; f) CD44; g) CD166; h) CD90; i) CD105; j) SH-3; k) SH-4; 1) TRA-1-60; m) TRA-1-81; n) SSEA-4 and; o) Sox-2.

Preferred methods include embodiments wherein said placental stem cell expresses OCT4.

Preferred methods include embodiments wherein said placental stem cell expresses Rex-1.

Preferred methods include embodiments wherein said placental stem cell expresses CD9.

Preferred methods include embodiments wherein said placental stem cell expresses CD29.

Preferred methods include embodiments wherein said placental stem cell expresses CD13.

Preferred methods include embodiments wherein said placental stem cell expresses CD44.

Preferred methods include embodiments wherein said placental stem cell expresses CD166.

Preferred methods include embodiments wherein said placental stem cell expresses CD90.

Preferred methods include embodiments wherein said placental stem cell expresses CD105.

Preferred methods include embodiments wherein said placental stem cell expresses SH3.

Preferred methods include embodiments wherein said placental stem cell expresses SH4.

Preferred methods include embodiments wherein said placental stem cell expresses TRA-1-60.

Preferred methods include embodiments wherein said placental stem cell expresses TRA-1-81.

Preferred methods include embodiments wherein said placental stem cell expresses SSEA4.

Preferred methods include embodiments wherein said placental stem cell expresses SOX-2.

Preferred methods include embodiments wherein said placental stem cell expresses IL-3 receptor.

Preferred methods include embodiments wherein said bone marrow stem cells comprise of bone marrow mononuclear cells.

Preferred methods include embodiments wherein said bone marrow mononuclear cells are isolated from bone marrow aspirate by use of a method selected from; a) gradient separation; b) erythrocyte lysis; and c) separation based on physical size.

Preferred methods include embodiments wherein said bone marrow stem cells are selected based on the ability to differentiate into mesenchymal stem cells.

Preferred methods include embodiments wherein said mesenchymal stem cells generated from said bone marrow cells are capable of differentiating into one or more cell types selected from a group comprising of; a) osteocytes; b) chondrocytes; and c) adipose tissue.

Preferred methods include embodiments wherein said bone marrow stem cells are selected based on expression of one or more of the following antigens: a) CD34; b) c-kit; c) flk-1; d) Stro-1; e) CD105; f) CD73; g) CD31; h) CD146; i) vascular endothelial-cadherin; j) CD133 and; k) CXCR-4.

Preferred methods include embodiments wherein said bone marrow stem cells express CD34.

Preferred methods include embodiments wherein said bone marrow stem cells express c-kit.

Preferred methods include embodiments wherein said bone marrow stem cells express flk-1.

Preferred methods include embodiments wherein said bone marrow stem cells express stro-1.

Preferred methods include embodiments wherein said bone marrow stem cells express CD105.

Preferred methods include embodiments wherein said bone marrow stem cells express CD73.

Preferred methods include embodiments wherein said bone marrow stem cells express CD31.

Preferred methods include embodiments wherein said bone marrow stem cells express CD146.

Preferred methods include embodiments wherein said bone marrow stem cells express vascular endothelial cadherin.

Preferred methods include embodiments wherein said bone marrow stem cells express CD133.

Preferred methods include embodiments wherein said bone marrow stem cells express CXCR-4.

Preferred methods include embodiments wherein said bone marrow stem cells are enriched for expression of CD133.

Preferred methods include embodiments wherein said amniotic fluid stem cells are isolated by introduction of a fluid extraction means into the amniotic cavity under ultrasound guidance.

Preferred methods include embodiments wherein said amniotic fluid stem cells are selected based on expression of one or more of the following antigens: a) SSEA3; b) SSEA4; c) Tra-1-60; d) Tra-1-81; e) Tra-2-54; f) HLA class I; g) CD13; h) CD44; i) CD49b; j) CD105; k) Oct-4; 1) Rex-1; m) DAZL and; n) Runx-1.

Preferred methods include embodiments wherein said amniotic fluid stem cells are selected based on lack of expression of one or more of the following antigens: a) CD34, b) CD45, and c) HLA Class II.

Preferred methods include embodiments wherein said cells possess the property of adherence to plastic and a mesenchymal-like morphology.

Preferred methods include embodiments wherein said neuronal stem cells are selected based on expression of one or more of the following antigens: a) RC-2; b) 3CB2; c) BLB; d) Sox-2hh; e) GLAST; f) Pax 6; g) nestin; h) Muashi-1; i) NCAM; j) A2B5; and k) prominin.

Preferred methods include embodiments wherein said neuronal stem cells are isolated from the dentate gyrus or the subventricular zone.

Preferred methods include embodiments wherein said circulating peripheral blood stem cells are characterized by ability to proliferate in vitro for a period of over 3 months.

Preferred methods include embodiments wherein said circulating peripheral blood stem cells are characterized by expression of a) CD34; b) CXCR4; c) CD117; d) CD113, and; e) c-met.

Preferred methods include embodiments wherein said circulating peripheral blood stem cell are expanded in vitro under conditions allowing for selective expansion of mesenchymal-like cells.

Preferred methods include embodiments wherein said circulating peripheral blood stem cells lack substantial expression of differentiation associated markers.

Preferred methods include embodiments wherein said differentiation associated markers are selected from a group comprising of a) CD2; b) CD3; c) CD4; d) CD11; e) CD11a; f) Mac-1; g) CD14; h) CD16; i) CD19; j) CD24; k) CD33; 1) CD36; m) CD38; n) CD45; o) CD56; p) CD64; q) CD68; r) CD86; s) CD66b; t) and; u) HLA-DR.

Preferred methods include embodiments wherein said mesenchymal stem cells express one or more of the following markers: a) STRO-1; b) CD105; c) CD54; d) CD106; e) HLA-I markers; f) vimentin; g) ASMA; h) collagen-1; i) fibronectin; j) LFA-3; k) ICAM-1; 1) PECAM-1; m) P-selectin; n) L-selectin; o) CD49b/CD29; p) CD49c/CD29; q) CD49d/CD29; r) CD61; s) CD18; t) CD29; u) thrombomodulin; v) telomerase; w) CD10; x) CD13; y) STRO-2; z) VCAM-1; aa) CD146 and; ab) THY-1.

Preferred methods include embodiments wherein said mesenchymal stem cells do not express substantial levels of a) HLA-DR; b) CD117; and c) CD45.

Preferred methods include embodiments wherein said mesenchymal stem cells are derived from a group selected of: a) bone marrow; b) adipose tissue; c) umbilical cord blood; d) Wharton's Jelly; e) enzymatically digested cord; f) inducible pluripotent generated cells; g) placental tissue; h) peripheral blood mononuclear cells; i) differentiated embryonic stem cells; and j) differentiated progenitor cells.

Preferred methods include embodiments wherein said germinal stem cells express markers selected from a group comprising of: a) Oct4; b) Nanog; c) Dppa5; d) Rbm; e) cyclin A2; f) Tex18; g) Stra8; h) Dazl; i) beta1- and alpha6-integrins; j) Vasa; k) Fragilis; 1) Nobox; m) c-Kit; n) Sca-1 and; o) Rex1.

Preferred methods include embodiments wherein said germinal stem cells are isolated from the testis.

Preferred methods include embodiments wherein said adipose tissue derived stem cells express markers selected from a group comprising of: a) CD13; b) CD29; c) CD44; d) CD63; e) CD73; f) CD90; g) CD166; h) Aldehyde dehydrogenase (ALDH); and i) ABCG2.

Preferred methods include embodiments wherein said adipose tissue derived stem cells are a population of purified mononuclear cells extracted from adipose tissue capable of proliferating in culture for more than 1 month.

Preferred methods include embodiments wherein said adipose tissue derived stem cells are plastic adherent.

Preferred methods include embodiments wherein said exfoliated teeth derived stem cells express markers selected from a group comprising of: a) STRO-1; b) CD146 (MUC18); c) alkaline phosphatase; d) MEPE; e) and; f) bFGF.

Preferred methods include embodiments wherein said hair follicle stem cells express markers selected from a group comprising of: a) cytokeratin 15; b) Nanog; and c) Oct-4.

Preferred methods include embodiments wherein said hair follicle stem cells are capable of proliferating in culture for a period of at least one month.

Preferred methods include embodiments wherein said hair follicle stem cells secrete one or more of the following proteins when grown in culture: a) basic fibroblast growth factor (bFGF); b) endothelin-1 (ET-1) and; c) stem cell factor (SCF).

Preferred methods include embodiments wherein said dermal stem cells express markers selected from a group comprising of: a) CD44; b) CD13; c) CD29; d) CD90; and e) CD105.

Preferred methods include embodiments wherein said dermal stem cells are capable of proliferating in culture for a period of at least one month.

Preferred methods include embodiments wherein said parthenogenically derived stem cells are generated by addition of a calcium flux inducing agent to activate an oocyte followed by enrichment of cells expressing markers selected from a group comprising of; a) SSEA-4; b) TRA 1-60; and c) TRA 1-81.

Preferred methods include embodiments wherein said reprogrammed stem cells are selected from a group comprising of: a) cells subsequent to a nuclear transfer; b) cells subsequent to a cytoplasmic transfer; c) cells treated with a DNA methyltransferase inhibitor; d) cells treated with a histone deacetylase inhibitor; e) cells treated with a GSK-3 inhibitor; f) cells induced to dedifferentiate by alteration of extracellular conditions; and g) cells treated with various combination of the mentioned treatment conditions.

Preferred methods include embodiments wherein said nuclear transfer comprises introducing nuclear material to a cell substantially enucleated, said nuclear material deriving from a host whose genetic profile is sought to be dedifferentiated.

Preferred methods include embodiments wherein said cytoplasmic transfer comprises introducing cytoplasm of a cell with a dedifferentiated phenotype into a cell with a differentiated phenotype, such that said cell with a differentiated phenotype substantially reverts to a dedifferentiated phenotype.

Preferred methods include embodiments wherein said DNA demethylating agent is selected from a group comprising of: a) 5-azacytidine; b) psammaplin A; and c) zebularine.

Preferred methods include embodiments wherein said histone deacetylase inhibitor is selected from a group comprising of: a) valproic acid; b) trichostatin-A; c) trapoxin A; and d) depsipeptide.

Preferred methods include embodiments wherein said endometrial regenerative cell is collected from menstrual blood based on selection for the marker c-kit.

Preferred methods include embodiments wherein said endometrial regenerative cell is collected from menstrual blood based on expansion of adherent cells in tissue culture.

Preferred methods include embodiments wherein said endometrial regenerative cell expresses markers selected from a group of proteins comprising of: a) CD90; b) CD105; and c) CD29.

Preferred methods include embodiments wherein said endometrial regenerative cell lacks substantial expression of markers selected from a group of proteins comprising of: a) HLA-I; b) CD14; and c) CD34.

Preferred methods include embodiments wherein said fallopian tube stem cells express markers selected from a group comprising of: a) CD29; b) CD44; c) CD90; d) CD13; e) CD73; f) SH2; g) SH3; and h) SH4.

Preferred methods include embodiments wherein said fallopian tube stem cells lacks substantial expression of markers selected from a group comprising of: a) CD34; b) CD38; c) CD45; d) CD117; e) CD133); CD31; and g) CD14.

Preferred methods include embodiments wherein said side population cells are identified based on expression multidrug resistance transport protein (ABCG2).

Preferred methods include embodiments wherein said side population cells are identified based on ability to efflux intracellular dyes.

Preferred methods include embodiments wherein said intracellular dyes may be rhodamine-123 or Hoechst 33342.

Preferred methods include embodiments wherein said side population is derived from a source of tissue, selected from; a) pancreatic tissue; b) liver tissue; c) smooth muscle tissue; d) striated muscle tissue; e) cardiac muscle tissue; f) bone tissue; g) bone marrow tissue; h) bone spongy tissue; i) cartilage tissue; j) liver tissue; k) pancreas tissue; 1) pancreatic ductal tissue; m) spleen tissue; n) thymus tissue; o) Peyer's patch tissue; p) lymph nodes tissue; q) thyroid tissue; r) epidermis tissue; s) dermis tissue; t) subcutaneous tissue; u) heart tissue; v) lung tissue; w) vascular tissue; x) endothelial tissue; y) blood cells; z) bladder tissue; aa) kidney tissue; ab) digestive tract tissue; ac) esophagus tissue; ad) stomach tissue; ae) small intestine tissue; af) large intestine tissue; ag) adipose tissue; ah) uterus tissue; ai) eye tissue; aj) lung tissue; ak) testicular tissue; al) ovarian tissue; am) prostate tissue; an) connective tissue; ao) endocrine tissue; ap) and mesentery tissue.

Preferred methods include embodiments wherein said umbilical cord derived mesenchymal cell expresses CD10.

Preferred methods include embodiments wherein said umbilical cord derived mesenchymal cell expresses CD24.

Preferred methods include embodiments wherein said umbilical cord derived mesenchymal cell expresses interleukin-11.

Preferred methods include embodiments wherein said umbilical cord derived mesenchymal cell expresses AIRE-1.

Preferred methods include embodiments wherein said umbilical cord derived mesenchymal cell expresses FoxP3.

Preferred methods include embodiments wherein said umbilical cord derived mesenchymal cell expresses angiopoietin-1.

Preferred methods include embodiments wherein said umbilical cord derived mesenchymal cell expresses CXCL1.

Preferred methods include embodiments wherein said umbilical cord derived mesenchymal cell expresses CD10 and CD73.

Preferred methods include embodiments wherein said umbilical cord derived mesenchymal cell expresses CD10 and CD133.

Preferred methods include embodiments wherein said umbilical cord derived mesenchymal cell expresses CD10 and CD90.

Preferred methods include embodiments wherein said umbilical cord derived mesenchymal cell expresses CD10 and CD274.

Preferred methods include embodiments wherein said umbilical cord derived mesenchymal cell expresses CD10 and AIRE.

Preferred methods include embodiments wherein said umbilical cord derived mesenchymal cell expresses CD10 and HLA-ABC.

Preferred methods include embodiments wherein said umbilical cord derived mesenchymal cell expresses CD10 and TLR-3.

Preferred methods include embodiments wherein said umbilical cord derived mesenchymal cell expresses CD10 and TLR-4.

Preferred methods include embodiments wherein said umbilical cord derived mesenchymal cell expresses CD10 and TLR-7.

Preferred methods include embodiments wherein said umbilical cord derived mesenchymal cell expresses CD10 and TLR-9.

Preferred methods include embodiments wherein said umbilical cord derived mesenchymal cell expresses CD10 and TLR-5.

Preferred methods include embodiments wherein said umbilical cord derived mesenchymal cell expresses CD10 and TLR-2.

Preferred methods include embodiments wherein said umbilical cord derived mesenchymal cell expresses CD10 and Stro-1.

Preferred methods include embodiments wherein mesenchymal stem cells are generated from pluripotent stem cells.

Preferred methods include embodiments wherein said mesenchymal stem cells are generated from pluripotent stem cells by a) culturing single cells in the presence of at least one growth factor in an amount sufficient to induce the differentiation of said clusters of cells into mesenchymal stem cells; b) adding one or more of basic fibroblast growth factor (bFGF), vascular endothelial growth factor (VEGF), bone morphogenic protein 4 (BMP-4), stem cell factor (SCF), Flt 3L (FL), thrombopoietin (TPO), EPO, and/or tPTD-HOXB4. The one or more of said at least one growth factor added in step (b) may be added to said culture within 36-60 hours from the start of step (a). Preferably, the one or more of said at least one growth factor added in step (b) is added to said culture within 40-48 hours from the start of step (a). The at least one factor added in step (b) may comprise one or more of bFGF, VEGF, BMP-4, SCF, FL and/or tPTD-HOXB4. The concentration of said growth factors if added in step (b) may range from about the following: bFGF is about 20-25 ng/ml, VEGF is about 20-100 ng/ml, BMP-4 is about 15-100 ng/ml, SCF is about 20-50 ng/ml, FL is about 10-50 ng/ml, TPO is about 20-50 ng/ml, and tPTD-HOXB4 is about 1.5-5 U/ml.

Preferred methods include embodiments wherein said subepithelial cord tissue stem cells are prepared by a process comprising: placing a subepithelial layer of a mammalian umbilical cord tissue in direct contact with a growth substrate; and culturing the subepithelial layer such that the isolated cell from the subepithelial layer is capable of self-renewal and culture expansion, wherein the isolated cell expresses at least three cell markers selected from the group consisting of CD29, CD73, CD90, CD166, SSEA4, CD9, CD44, CD146, or CD105, and wherein the isolated cell does not express NANOG and at least five cell markers selected from the group consisting of CD45, CD34, CD14, CD79, CD106, CD86, CD80, CD19, CD117, Stro-1, or HLA-DR.

Preferred methods include embodiments wherein said cells expresses CD29, CD73, CD90, CD166, SSEA4, CD9, CD44, CD146, and CD105.

Preferred methods include embodiments wherein said cells express CD105.

Preferred methods include embodiments wherein said cells express CD146.

Preferred methods include embodiments wherein said cells express CD44.

Preferred methods include embodiments wherein said cells express CD9.

Preferred methods include embodiments wherein said cells express SSEA4.

Preferred methods include embodiments wherein said cells express CD166.

Preferred methods include embodiments wherein said cells express CD90.

Preferred methods include embodiments wherein said cells expresses CD73.

Preferred methods include embodiments wherein said cells expresses CD29.

Preferred methods include embodiments wherein said cells do not express CD45, CD34, CD14, CD79, CD106, CD86, CD80, CD19, CD117, Stro-1, and HLA-DR.

Preferred methods include embodiments wherein said cells are positive for SOX2.

Preferred methods include embodiments wherein said cells are positive for OCT4.

Preferred methods include embodiments wherein said cells are positive for SOX2 and OCT4.

Preferred methods include embodiments wherein said cells are capable of differentiation into a cell type selected from the group consisting of adipocytes, chondrocytes, osteocytes, cardiomyocytes, endothelial cells, and myocytes.

Preferred methods include embodiments wherein said cells are treated with an agent capable of enhancing anti-inflammatory activity of said cells and/or products generated by said cells.

Preferred methods include embodiments wherein said products generated by said cells are cytokines.

Preferred methods include embodiments wherein said cytokine is interleukin 1 receptor antagonist.

Preferred methods include embodiments wherein said cytokine is interleukin 3.

Preferred methods include embodiments wherein said cytokine is interleukin 4.

Preferred methods include embodiments wherein said cytokine is interleukin 6.

Preferred methods include embodiments wherein said cytokine is interleukin 7.

Preferred methods include embodiments wherein said cytokine is interleukin 10.

Preferred methods include embodiments wherein said cytokine is interleukin 13.

Preferred methods include embodiments wherein said cytokine is interleukin 16.

Preferred methods include embodiments wherein said cytokine is interleukin 19.

Preferred methods include embodiments wherein said cytokine is interleukin 20.

Preferred methods include embodiments wherein said cytokine is interleukin 22.

Preferred methods include embodiments wherein said cytokine is interleukin 30.

Preferred methods include embodiments wherein said cytokine is interleukin 35.

Preferred methods include embodiments wherein said cytokine is prostaglandin E1.

Preferred methods include embodiments wherein said cytokine is prostaglandin E2.

Preferred methods include embodiments wherein said cytokine is interferon beta.

Preferred methods include embodiments wherein said cytokine is soluble HLA-G.

Preferred methods include embodiments wherein said cytokine is TGF-beta.

Preferred methods include embodiments wherein said cytokine is endoglin.

Preferred methods include embodiments wherein said cytokine is VEGF.

Preferred methods include embodiments wherein said cytokine is placental growth factor.

Preferred methods include embodiments wherein said cytokine is leukemia inhibitory factor.

Preferred methods include embodiments wherein said cytokine is FGF-1.

Preferred methods include embodiments wherein said cytokine is FGF-2.

Preferred methods include embodiments wherein said cytokine is EGF-1.

Preferred methods include embodiments wherein said cytokine is IGF-1

Preferred methods include embodiments wherein said cytokine is HGF-1.

Preferred methods include embodiments wherein said cytokine is IGF-binding protein.

Preferred methods include embodiments wherein said cytokine is prostaglandin E1.

Preferred methods include embodiments wherein said products generated by said cells are exosomes.

Preferred methods include embodiments wherein said exosomes express annexin V.

Preferred methods include embodiments wherein said exosomes are cup-shaped.

Preferred methods include embodiments wherein said exosomes inhibit macrophage activation.

Preferred methods include embodiments wherein said macrophage activation is production of nitric oxide in response to stimulation with lipopolysaccharide.

Preferred methods include embodiments wherein said macrophage activation is production of nitric oxide in response to stimulation with beta glucan.

Preferred methods include embodiments wherein said macrophage activation is production of nitric oxide in response to stimulation with Poly IC.

Preferred methods include embodiments wherein said macrophage activation is production of nitric oxide in response to stimulation with CpG DNA.

Preferred methods include embodiments wherein said macrophage activation is production of nitric oxide in response to stimulation with HMGB1.

Preferred methods include embodiments wherein said macrophage activation is production of nitric oxide in response to stimulation with neutrophil extracellular traps.

Preferred methods include embodiments wherein said macrophage activation is production of nitric oxide in response to stimulation with interferon gamma.

Preferred methods include embodiments, wherein said macrophage activation is production of nitric oxide in response to stimulation with allogeneic T cells.

Preferred methods include embodiments wherein said macrophage activation is production of nitric oxide in response to stimulation with syngeneic activated T cells.

Preferred methods include embodiments wherein said agent capable of stimulate anti-inflammatory activity is a cytokine.

Preferred methods include embodiments wherein said cytokine is an inflammatory cytokine.

Preferred methods include embodiments wherein said inflammatory cytokine is capable of activating NF-kappa b in a regenerative cell population.

Preferred methods include embodiments wherein said inflammatory cytokine is capable of activating NF-kappa b in a stem cell population.

Preferred methods include embodiments wherein said inflammatory cytokine is capable of activating NF-kappa b in a mesenchymal stem cell population.

Preferred methods include embodiments wherein said inflammatory cytokine is capable of activating NF-kappa b in a fibroblast cell population.

Preferred methods include embodiments wherein said inflammatory cytokine is capable of activating NF-kappa b in an endothelial cell population.

Preferred methods include embodiments wherein said inflammatory cytokine is capable of activating NF-kappa b in a monocytic cell population.

Preferred methods include embodiments wherein said inflammatory cytokine is capable of activating HIF-1 alpha in a regenerative cell population.

Preferred methods include embodiments wherein said inflammatory cytokine is capable of activating HIF-1 alpha in a stem cell population.

Preferred methods include embodiments wherein said inflammatory cytokine is capable of activating HIF-1 alpha in a mesenchymal stem cell population.

Preferred methods include embodiments wherein said inflammatory cytokine is capable of activating HIF-1 alpha in a fibroblast cell population.

Preferred methods include embodiments wherein said inflammatory cytokine is capable of activating HIF-1 alpha in an endothelial cell population.

Preferred methods include embodiments wherein said inflammatory cytokine is capable of activating HIF-1 alpha in a monocytic cell population.

Preferred methods include embodiments wherein agent capable of increasing anti-inflammatory activity in said regenerative cell is PGE-1.

Preferred methods include embodiments wherein agent capable of increasing anti-inflammatory activity in said regenerative cell is PGE-2.

Preferred methods include embodiments wherein agent capable of increasing anti-inflammatory activity in said regenerative cell is TGF-beta.

Preferred methods include embodiments wherein agent capable of increasing anti-inflammatory activity in said regenerative cell is TGF-alpha.

Preferred methods include embodiments wherein agent capable of increasing anti-inflammatory activity in said regenerative cell is PDGF-1.

Preferred methods include embodiments wherein agent capable of increasing anti-inflammatory activity in said regenerative cell is PDGF-BB.

Preferred methods include embodiments wherein agent capable of increasing anti-inflammatory activity in said regenerative cell is endothelin-1.

Preferred methods include embodiments wherein agent capable of increasing anti-inflammatory activity in said regenerative cell is NGF.

Preferred methods include embodiments, wherein agent capable of increasing anti-inflammatory activity in said regenerative cell is BDNF.

Preferred methods include embodiments wherein agent capable of increasing anti-inflammatory activity in said regenerative cell is TRAIL.

Preferred methods include embodiments wherein agent capable of increasing anti-inflammatory activity in said regenerative cell is angiopoietin.

Preferred methods include embodiments wherein agent capable of increasing anti-inflammatory activity in said regenerative cell is TNF-alpha.

Preferred methods include embodiments wherein agent capable of increasing anti-inflammatory activity in said regenerative cell is TNF-beta.

Preferred methods include embodiments wherein agent capable of increasing anti-inflammatory activity in said regenerative cell is serotonin.

Preferred methods include embodiments wherein agent capable of increasing anti-inflammatory activity in said regenerative cell is dopamine.

Preferred methods include embodiments wherein agent capable of increasing anti-inflammatory activity in said regenerative cell is astralagus extract.

Preferred methods include embodiments wherein agent capable of increasing anti-inflammatory activity in said regenerative cell is interleukin-1.

Preferred methods include embodiments wherein agent capable of increasing anti-inflammatory activity in said regenerative cell is interleukin-2.

Preferred methods include embodiments wherein agent capable of increasing anti-inflammatory activity in said regenerative cell is interleukin-4.

Preferred methods include embodiments wherein agent capable of increasing anti-inflammatory activity in said regenerative cell is interleukin-6.

Preferred methods include embodiments wherein agent capable of increasing anti-inflammatory activity in said regenerative cell is interleukin-10.

Preferred methods include embodiments wherein agent capable of increasing anti-inflammatory activity in said regenerative cell is interleukin-9.

Preferred methods include embodiments wherein agent capable of increasing anti-inflammatory activity in said regenerative cell is interleukin-11.

Preferred methods include embodiments wherein agent capable of increasing anti-inflammatory activity in said regenerative cell is interleukin-12.

Preferred methods include embodiments wherein agent capable of increasing anti-inflammatory activity in said regenerative cell is interleukin-13.

Preferred methods include embodiments, wherein agent capable of increasing anti-inflammatory activity in said regenerative cell is interleukin-15.

Preferred methods include embodiments wherein agent capable of increasing anti-inflammatory activity in said regenerative cell is interleukin-17.

Preferred methods include embodiments wherein agent capable of increasing anti-inflammatory activity in said regenerative cell is interleukin-18.

Preferred methods include embodiments wherein agent capable of increasing anti-inflammatory activity in said regenerative cell is interleukin-19.

Preferred methods include embodiments wherein agent capable of increasing anti-inflammatory activity in said regenerative cell is interleukin-20.

Preferred methods include embodiments wherein agent capable of increasing anti-inflammatory activity in said regenerative cell is interleukin-21.

Preferred methods include embodiments wherein agent capable of increasing anti-inflammatory activity in said regenerative cell is interleukin-22.

Preferred methods include embodiments wherein agent capable of increasing anti-inflammatory activity in said regenerative cell is interleukin-23.

Preferred methods include embodiments wherein agent capable of increasing anti-inflammatory activity in said regenerative cell is interleukin-25.

Preferred methods include embodiments wherein agent capable of increasing anti-inflammatory activity in said regenerative cell is interleukin-27.

Preferred methods include embodiments wherein agent capable of increasing anti-inflammatory activity in said regenerative cell is interleukin-33.

Preferred methods include embodiments wherein agent capable of increasing anti-inflammatory activity in said regenerative cell is interleukin-35.

Preferred methods include embodiments wherein agent capable of increasing anti-inflammatory activity in said regenerative cell is interferon alpha.

Preferred methods include embodiments wherein agent capable of increasing anti-inflammatory activity in said regenerative cell is interferon beta.

Preferred methods include embodiments wherein agent capable of increasing anti-inflammatory activity in said regenerative cell is interferon gamma.

Preferred methods include embodiments wherein agent capable of increasing anti-inflammatory activity in said regenerative cell is interferon tau.

Preferred methods include embodiments wherein agent capable of increasing anti-inflammatory activity in said regenerative cell is interferon omega.

Preferred methods include embodiments wherein agent capable of increasing anti-inflammatory activity in said regenerative cell is an mTOR inhibitor.

Preferred methods include embodiments wherein said mTOR inhibitor is everolimus.

Preferred methods include embodiments wherein said mTOR inhibitor is sirolimus.

Preferred methods include embodiments wherein said mTOR inhibitor is temsirolimus.

Preferred methods include embodiments wherein said mTOR inhibitor is dactolisib.

Preferred methods include embodiments wherein said mTOR inhibitor is GSK2126458.

Preferred methods include embodiments wherein said mTOR inhibitor is XL765.

Preferred methods include embodiments wherein said mTOR inhibitor is AZD8055.

Preferred methods include embodiments wherein said mTOR inhibitor is INK128.

Preferred methods include embodiments wherein said mTOR inhibitor is MLN0128.

Preferred methods include embodiments wherein said mTOR inhibitor is OSI0271.

Preferred methods include embodiments wherein said mTOR inhibitor is RapaLinks.

Preferred methods include embodiments wherein agent capable of increasing anti-inflammatory activity in said regenerative cell is an activator of a pattern recognition receptor.

Preferred methods include embodiments wherein said activator of said pattern recognition receptor is a toll like receptor agonist.

Preferred methods include embodiments wherein said toll like receptor is an immune receptor.

Preferred methods include embodiments wherein said immune receptor is TLR. 1

Preferred methods include embodiments wherein said TLR-1 is activated by Pam3CSK4.

Preferred methods include embodiments wherein said immune receptor is TLR-2

Preferred methods include embodiments wherein said TLR-2 is activated by HKLM.

Preferred methods include embodiments wherein said immune receptor is TLR-3.

Preferred methods include embodiments wherein said TLR-3 is activated by Poly:IC.

Preferred methods include embodiments wherein said immune receptor is TLR-4.

Preferred methods include embodiments wherein said TLR-4 is activated by LPS.

Preferred methods include embodiments wherein said TLR-4 is activated by Buprenorphine.

Preferred methods include embodiments wherein said TLR-4 is activated by Carbamazepine.

Preferred methods include embodiments wherein said TLR-4 is activated by Fentanyl.

Preferred methods include embodiments wherein said TLR-4 is activated by Levorphanol.

Preferred methods include embodiments wherein said TLR-4 is activated by Methadone.

Preferred methods include embodiments wherein said TLR-4 is activated by Cocaine.

Preferred methods include embodiments wherein said TLR-4 is activated by Morphine.

Preferred methods include embodiments wherein said TLR-4 is activated by Oxcarbazepine.

Preferred methods include embodiments wherein said TLR-4 is activated by Oxycodone.

Preferred methods include embodiments wherein said TLR-4 is activated by Pethidine.

Preferred methods include embodiments wherein said TLR-4 is activated by Glucuronoxylomannan from Cryptococcus.

Preferred methods include embodiments wherein said TLR-4 is activated by Morphine-3-glucuronide.

Preferred methods include embodiments wherein said TLR-4 is activated by lipoteichoic acid.

Preferred methods include embodiments wherein said TLR-4 is activated by beta.-defensin 2.

Preferred methods include embodiments wherein said TLR-4 is activated by low molecular weight hyaluronic acid.

Preferred methods include embodiments wherein said low molecular weight hyaluronic acid has a molecular weight of <1000 kDa.

Preferred methods include embodiments wherein said low molecular weight hyaluronic acid has a molecular weight of <500 kDa.

Preferred methods include embodiments wherein said low molecular weight hyaluronic acid has a molecular weight of <250 kDa.

Preferred methods include embodiments wherein said low molecular weight hyaluronic acid has a molecular weight of <100 kDa.

Preferred methods include embodiments wherein said TLR-4 is activated by fibronectin EDA.

Preferred methods include embodiments wherein said TLR-4 is activated by snapin.

Preferred methods include embodiments wherein said TLR-4 is activated by tenascin C.

Preferred methods include embodiments wherein said immune receptor is TLR-5.

Preferred methods include embodiments wherein said TLR-5 is activated by flaggelin.

Preferred methods include embodiments wherein said immune receptor is TLR-6.

Preferred methods include embodiments wherein said TLR-6 is activated by FSL-1.

Preferred methods include embodiments wherein said immune receptor is TLR-7.

Preferred methods include embodiments wherein said TLR-7 is activated by imiquimod.

Preferred methods include embodiments, wherein said immune receptor is TLR-8.

Preferred methods include embodiments wherein said TLR-8 is activated by ssRNA40/LyoVec.

Preferred methods include embodiments wherein said immune receptor is TLR-9.

Preferred methods include embodiments wherein said TLR-9 is activated by a CpG oligonucleotide.

Preferred methods include embodiments wherein said TLR-9 is activated by ODN2006.

Preferred methods include embodiments wherein said TLR-9 is activated by Agatolimod.

Preferred methods include embodiments wherein said TLR-9 is activated by ODN2007.

Preferred methods include embodiments wherein said TLR-9 is activated by ODN1668.

Preferred methods include embodiments wherein said TLR-9 is activated by ODN1826.

Preferred methods include embodiments wherein said TLR-9 is activated by ODN BW006.

Preferred methods include embodiments wherein said TLR-9 is activated by ODN D SL01.

Preferred methods include embodiments wherein said TLR-9 is activated by ODN 2395.

Preferred methods include embodiments wherein said TLR-9 is activated by ODN M362.

Preferred methods include embodiments wherein said TLR-9 is activated by ODN SLO3

Preferred methods include embodiments wherein said monocytic population is cultured in the presence of a regenerative cells together with an activation stimuli.

Preferred methods include embodiments wherein said activation stimuli is an antibody.

Preferred methods include embodiments wherein said antibody induces NF-kappa expression in said monocytic cell population.

Preferred methods include embodiments wherein said antibody crosslinks CD14.

Preferred methods include embodiments wherein said antibody crosslinks CD2.

Preferred methods include embodiments wherein said antibody crosslinks CD1 lb.

Preferred methods include embodiments wherein said antibody crosslinks CD16.

Preferred methods include embodiments wherein said antibody crosslinks CD31.

Preferred methods include embodiments wherein said antibody crosslinks CD56.

Preferred methods include embodiments wherein said antibody crosslinks CD57.

Preferred methods include embodiments wherein said antibody crosslinks CD62 ligand.

Preferred methods include embodiments wherein said antibody crosslinks CD115.

Preferred methods include embodiments wherein said antibody crosslinks CD192.

Preferred methods include embodiments wherein said antibody crosslinks TNF receptor p55.

Preferred methods include embodiments wherein said antibody crosslinks TNF receptor p75.

Preferred methods include embodiments wherein said antibody crosslinks CX3CR1.

Preferred methods include embodiments wherein said antibody crosslinks CXCR3.

Preferred methods include embodiments wherein said antibody crosslinks CXCR4.

Preferred methods include embodiments wherein said antibody crosslinks interferon gamma receptor.

Preferred methods include embodiments wherein said antibody crosslinks interleukin 1 receptor.

Preferred methods include embodiments wherein said antibody crosslinks interleukin 3 receptor.

Preferred methods include embodiments wherein said antibody crosslinks interleukin 4 receptor.

Preferred methods include embodiments wherein said antibody crosslinks interleukin 6 receptor.

Preferred methods include embodiments wherein said antibody crosslinks interleukin 8 receptor.

Preferred methods include embodiments wherein said antibody crosslinks interleukin 9 receptor.

Preferred methods include embodiments wherein said antibody crosslinks interleukin 10 receptor.

Preferred methods include embodiments wherein said antibody crosslinks interleukin 12 receptor.

Preferred methods include embodiments wherein said antibody crosslinks interleukin 13 receptor.

Preferred methods include embodiments wherein said antibody crosslinks interleukin 15 receptor.

Preferred methods include embodiments wherein said antibody crosslinks interleukin 17 receptor.

Preferred methods include embodiments wherein said antibody crosslinks interleukin 18 receptor.

Preferred methods include embodiments wherein said antibody crosslinks interleukin 20 receptor.

Preferred methods include embodiments wherein said antibody crosslinks interleukin 21 receptor.

Preferred methods include embodiments wherein said antibody crosslinks interleukin 22 receptor.

Preferred methods include embodiments wherein said antibody crosslinks interleukin 24 receptor.

Preferred methods include embodiments wherein said antibody crosslinks interleukin 27 receptor.

Preferred methods include embodiments wherein said antibody crosslinks interleukin 29 receptor.

Preferred methods include embodiments wherein said antibody crosslinks interleukin 30 receptor.

Preferred methods include embodiments wherein said antibody crosslinks interleukin 33 receptor.

Preferred methods include embodiments wherein said monocytic population is cultured in the presence of a regenerative cells together with an adjuvant which increases the regenerative and/or immunomodulatory activities of said monocytic population.

Preferred methods include embodiments wherein said adjuvant is a T regulatory cell.

Preferred methods include embodiments wherein said T regulatory cell expresses foxp-3.

Preferred methods include embodiments wherein said T regulatory cell expresses Helios.

Preferred methods include embodiments wherein said T regulatory cell expresses STATS.

Preferred methods include embodiments wherein said T regulatory cell expresses CD5.

Preferred methods include embodiments wherein said T regulatory cell expresses CD25.

Preferred methods include embodiments wherein said T regulatory cell expresses CD39.

Preferred methods include embodiments wherein said T regulatory cell expresses CD105.

Preferred methods include embodiments wherein said T regulatory cell expresses IL-7 receptor.

Preferred methods include embodiments wherein said T regulatory cell expresses CTLA-4.

Preferred methods include embodiments wherein said T regulatory cell expresses folate receptor.

Preferred methods include embodiments wherein said T regulatory cell expresses CD223

Preferred methods include embodiments wherein said T regulatory cell expresses LAP.

Preferred methods include embodiments wherein said T regulatory cell expresses GARP.

Preferred methods include embodiments wherein said T regulatory cell expresses Neuropilin.

Preferred methods include embodiments wherein said T regulatory cell expresses CD134.

Preferred methods include embodiments wherein said T regulatory cell expresses CD62 ligand.

Preferred methods include embodiments wherein said T regulatory cell secretes IL-10.

Preferred methods include embodiments wherein said T regulatory cell secretes TGF-alpha.

Preferred methods include embodiments wherein said T regulatory cell secretes TGF-beta.

Preferred methods include embodiments wherein said T regulatory cell secretes soluble TNF receptor p55.

Preferred methods include embodiments wherein said T regulatory cell secretes soluble TNF receptor p75.

Preferred methods include embodiments wherein said T regulatory cell secretes IL-2.

Preferred methods include embodiments wherein said T regulatory cell secretes soluble HLA-G.

Preferred methods include embodiments wherein said T regulatory cell secretes soluble Fas ligand.

Preferred methods include embodiments wherein said T regulatory cell secretes IL-35.

Preferred methods include embodiments wherein said T regulatory cell secretes VEGF.

Preferred methods include embodiments wherein said T regulatory cell secretes HGF.

Preferred methods include embodiments wherein said T regulatory cell secretes FGF1.

Preferred methods include embodiments wherein said T regulatory cell secretes FGF2.

Preferred methods include embodiments wherein said T regulatory cell secretes FGF5.

Preferred methods include embodiments wherein said T regulatory cell secretes Galectin 1.

Preferred methods include embodiments wherein said T regulatory cell secretes Galectin 9.

Preferred methods include embodiments wherein said T regulatory cell secretes IL-20.

Preferred methods include embodiments wherein said T regulatory cell expresses perforin.

Preferred methods include embodiments wherein said T regulatory cell expresses granzyme.

Preferred methods include embodiments wherein said T regulatory cell inhibits activation of a conventional T cell.

Preferred methods include embodiments wherein said adjuvant is TGF-beta.

Preferred methods include embodiments wherein said adjuvant is IL-10.

Preferred methods include embodiments wherein said adjuvant is leukemia inhibitory factor.

Preferred methods include embodiments wherein said adjuvant is PDGF-BB.

Preferred methods include embodiments wherein said adjuvant is IVIG.

Preferred methods include embodiments wherein said adjuvant is human chorionic gonadotrophin.

Preferred methods include embodiments wherein said adjuvant is valproic acid.

Preferred methods include embodiments wherein said adjuvant is 5-azacytidine.

Preferred methods include embodiments wherein said adjuvant is lithium or a salt thereof.

Preferred methods include embodiments wherein said adjuvant is platelet rich plasma.

Preferred methods include embodiments wherein said monocyte subsequent to said contact with regenerative cell is altered to maintain a level of resistance against inflammatory effects.

Preferred methods include embodiments wherein said cells are gene edited to lack expression of inflammasome related genes.

Preferred methods include embodiments wherein said inflammasome related gene is ASC.

Preferred methods include embodiments wherein said inflammasome related gene is CASP1.

Preferred methods include embodiments wherein said inflammasome related gene is GSDMA.

Preferred methods include embodiments wherein said inflammasome related gene is GSDMB.

Preferred methods include embodiments wherein said inflammasome related gene is GSDMC.

Preferred methods include embodiments wherein said inflammasome related gene is GSDMD.

Preferred methods include embodiments wherein said inflammasome related gene is GSDME.

Preferred methods include embodiments wherein said inflammasome related gene is GSDMF.

Preferred methods include embodiments wherein said inflammasome related gene is HMGB1.

Preferred methods include embodiments wherein said inflammasome related gene is CARD1.

Preferred methods include embodiments wherein said inflammasome related gene is NLRP3.

Preferred methods include embodiments wherein said cells treated in a manner to allow for induction of RNA interference such that suppression of inflammasome gene expression is induced.

Preferred methods include embodiments wherein said induction of RNA interference is accomplished by administration of short interfering RNA.

Preferred methods include embodiments wherein said induction of RNA interference is accomplished by administration of hairpin loop RNA.

Preferred methods include embodiments wherein said inflammasome related gene is ASC.

Preferred methods include embodiments wherein said inflammasome related gene is CASP1.

Preferred methods include embodiments wherein said inflammasome related gene is GSDMA.

Preferred methods include embodiments wherein said inflammasome related gene is GSDMB.

Preferred methods include embodiments wherein said inflammasome related gene is GSDMC.

Preferred methods include embodiments wherein said inflammasome related gene is GSDMD.

Preferred methods include embodiments wherein said inflammasome related gene is GSDME.

Preferred methods include embodiments wherein said inflammasome related gene is GSDMF.

Preferred methods include embodiments wherein said inflammasome related gene is HMGB1.

Preferred methods include embodiments wherein said inflammasome related gene is CARD1.

Preferred methods include embodiments wherein said inflammasome related gene is NLRP3.

### DETAILED DESCRIPTION OF THE ILLUSTRATED EMBODIMENTS

Embodiments of the present invention are described below. It is, however, expressly noted that the present invention is not limited to these embodiments, but rather the intention is that modifications that are apparent to the person skilled in the art and equivalents thereof are also included.

In one embodiment the invention provides the generation of myeloid cells which possess immune suppressive and/or immune modulatory properties. In some embodiments of the invention, culture of conditioned media with patient adherent cells is performed. Adherent cells can be substituted with cells selected for monocytic lineages. In some embodiments, culture media from regenerative cells is utilized to augment generation of myeloid derived suppressor cells using protocols that have already been disclosed. For example, monocytes cultured with regenerative cell conditioned media are also treated/transfected with immunoglobulin-like transcript 3 (ILT3, also known as LILRB4) and immunoglobulin-like transcript 4 (ILT4, also known as LILRB2) [19-32], interleukin-10 [33, 34], aspirin [35], niflumic acid [36],

“Adaptive immunity” is described as T and B cell immune responses work together with innate immune responses. The basis of the adaptive immune response is that of clonal recognition and response. An antigen selects the clones of cell which recognize it, and the first element of a specific immune response must be rapid proliferation of the specific lymphocytes. This is followed by further differentiation of the responding cells as the effector phase of the immune response develops. In T-cell mediated non-infective inflammatory diseases and conditions, immunosuppressive drugs inhibit T-cell proliferation and block their differentiation and effector functions.

“T cell response” means an immunological response involving T cells. The T cells that are “activated” divide to produce memory T cells or cytotoxic T cells. The cytotoxic T cells bind to and destroy cells recognized as containing the antigen. The memory T cells are activated by the antigen and thus provide a response to an antigen already encountered. This overall response to the antigen is the T cell response.

“autoimmune disease” or “autoimmune response” is a response in which the immune system of an individual initiates and may propagate a primary and/or secondary response against its own tissues or cells. An “alloimmune response” is one in which the immune system of an individual initiates and may propagate a primary and/or secondary response against the tissues, cells, or molecules of another, as, for example, in a transplant or transfusion.

The term “cell-mediated immunity” refers to (1) the recognition and/or killing of virus and virus-infected cells by leukocytes and (2) the production of different soluble factors (cytokines) by these cells when stimulated by virus or virus-infected cells. Cytotoxic T lymphocytes (CTLs), natural killer (NK) cells and antiviral macrophages are leukocytes that can recognize and kill virus-infected cells. Helper T cells can recognize virus-infected cells and produce a number of important cytokines. Cytokines produced by monocytes (monokines), T cells, and NK cells (lymphokines) play important roles in regulating immune functions and developing antiviral immune functions. A host T cell response can be directed against cells of the host, as in autoimmune disease. For example, the T cells in type I diabetes (T1D) recognize an “antigen” that is expressed by the host, which causes the destruction of normal host cells—for T1D, the endocrine cells of the islets of Langerhans of the pancreas. A T cell response may also occur within a host that has received a graft of foreign cells, as is the case in graft-versus-host disease (GVHD) in which T cells from the graft attack the cells of the host, or in the case of graft rejection in which T cells of the host attack the graft.

“T regulatory cell” or “Treg cell” or “Tr cell” refers to a cell that can inhibit a T cell response [37-41]. Treg cells express the transcription factor Foxp3, which is not upregulated upon T cell activation and discriminates Tregs from activated effector cells [42]. Tregs are identified by the cell surface markers CD25, CD45RB, CTLA4, and GITR. Treg development is induced by MDSC activity [43]. Several Treg subsets have been identified that have the ability to inhibit autoimmune and chronic inflammatory responses and to maintain immune tolerance in tumor-bearing hosts [44]. These subsets include interleukin 10-(IL-10-) secreting T regulatory type 1 (Tr1) cells, transforming growth factor-.beta.-(TGF-.beta.-) secreting T helper type 3 (Th3) cells, and “natural” CD4. sup.+/CD25. sup.+ Tregs (Trn).

The phrase “inducing T regulatory cells” means activation, amplification, and generation of Tregs to inhibit or reduce the T cell response. One method of induction is through the use of the MDSCs.

The phrase “T cell tolerance” refers to the anergy (non-responsiveness) of T cells when presented with an antigen. T cell tolerance prevents a T cell response even in the presence of an antigen that existing memory T cells recognize.

“Differentiate” refers to the genetic process by which cells are produced with a specialized phenotype. A differentiated cell of any type has attained all of the characteristics that define that cell type. This is true even in the progression of cell types. For example, if cell type X matures to cell type Y which then overall matures to cell type Z, an X cell differentiates to a Y cell when it has attained all of the characteristics that define a type Y cell, even though the cell has not completely differentiated into a type Z cell.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V.sub.H) and a heavy chain constant region. The heavy chain constant region is comprised of three domains, C.sub.H1, C. sub.H2 and C.sub.H3. Each light chain is comprised of a light chain variable region (abbreviated herein as V.sub.L) and a light chain constant region. The light chain constant region is comprised of one domain, C.sub.L. The V.sub.H and V.sub.L regions can be further subdivided into regions of hypervariability, termed complementarity determining regions (CDR), interspersed with regions that are more conserved, termed framework regions (FR). Each V.sub.H and V.sub.L is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

“Cytokine” is a generic term for a group of proteins released by one cell population which act on another cell population as intercellular mediators. Examples of such cytokines are lymphokines, monokines, and traditional polypeptide hormones. Included among the cytokines are interferons (IFN, notably IFN-.gamma.), interleukins (IL, notably IL-1, IL-2, IL-4, IL-10, IL-12), colony stimulating factors (CSF), macrophage colony stimulating factor (M-CSF), granulocyte macrophage colony stimulating factor (GM-CSF), thrombopoietin (TPO), erythropoietin (EPO), leukemia inhibitory factor (LIF), kit-ligand, growth hormones (GH), insulin-like growth factors (IGF), parathyroid hormone, thyroxine, insulin, relaxin, follicle stimulating hormone (FSH), thyroid stimulating hormone (TSH), leutinizing hormone (LH), hematopoietic growth factor, hepatic growth factor, fibroblast growth factors (FGF), prolactin, placental lactogen, tumor necrosis factors (TNF), mullerian-inhibiting substance, mouse gonadotropin-associated peptide, inhibin, activin, vascular endothelial growth factor (VEGF), integrin, nerve growth factors (NGF), platelet growth factor, transforming growth factors (TGF), osteoinductive factors, etc. Those of particular interest for the present invention include IFN-.gamma., IL-10, and TGF-.beta.

“Autoantigen” refers to a molecule that is endogenous to a cell or organism that induces an autoimmune response.

“Transplant rejection” means that a transplant of tissue or cells is not tolerated by a host individual. The transplant is not tolerated in that it is attacked by the host's own immune system or is otherwise not supported by the host. The transplant may be an allotransplant, a transplant of tissue or cells from another individual of the same species, or an autotransplant, a transplant of the host's own tissue or cells. Transplant rejection encompasses the rejection of fluids through transfusion.

The term “subject” or “individual” as used herein refers to an animal having an immune system, preferably a mammal (e.g., rodent such as mouse). In particular, the term refers to humans.

The immune suppressive properties of myeloid derived suppressor cells have been well described in the literature and one of skill in the art is referred to various relevant publication for better understanding and practice of the invention. Myeloid derived suppressor cells have been implicated in tuberculosis infection [45, 46], cancer [47-64], transplantation tolerance [65-71], graft versus host disease [72].

Manipulation of myeloid derived suppressor cell activity has been previously used therapeutically in that suppression of their activity by agents such as PDES inhibitors [73], all-trans-retinoic acid [74], amino-bisphosphonates [75], stat 3 inhibitors [76], triterpenoids [46, 77], 5-flourouracil [78], cox-2 inhibitors [79], have been used for immune stimulation. In contrast, the current invention teaches means of enhancing myeloid derived suppressor activity in order to induce immunological tolerance to diabetogenic antigens and/or to protect pancreatic islet cells from death. In some embodiments the invention uses patient lymphocytes conditioned by stem cells to increase TGF-beta expression on myeloid suppressor cells, thereby increasing potency of myeloid suppressor cell inhibition of immunity [80]. In some embodiments of the invention activation of IL-4 receptor myeloid derived suppressor cells [81], is disclosed through administration of patient lymphocytes that have been conditioned with regenerative cells.

In one embodiment of the invention, compounds that stimulate activity of myeloid derived suppressor cells are given along with the regenerative cell reprogrammed PBMC. In one embodiment compounds such as interleukin-6 [82-85], PGE-2 [86], S100A9 [87], exosomes [88, 89], LPS and interferon gamma [90], GM-CSF [91, 92], IL-6 [93], M-CSF [94], BCG [95], alcohol consumption [96], TLR-2 activators [97], hepatic acute phase proteins such as serum amyloid A and Cxcll/K [98], Galectin-9 [99], anti-CD137 antibodies [93] re administered to augment activity of myeloid derived suppressor cells. In some embodiments, enhancement of myeloid derived suppressor cell function such as increasing arginine metabolism [100], is accomplished by activation of said cells with agent such as toll like receptor activators.

In one embodiment of the invention, IL-17 producing gamma delta T cells are utilized to generate myeloid suppressor cells which can be used in the context of the invention to prevent or reverse diabetes. The utilization of IL-17 producing gamma delta T cells to generate myeloid suppressor cells is described [101-105]. Additionally, simple IL-17 administration either directly, or through administration of cells secreting IL-17 may be used for stimulation of myeloid derived suppressor cells [106, 107]. In some embodiments of the invention, myeloid derived suppressor cells are utilized to kill NK cells [108], wherein said ex vivo conditioned patient lymphocytes are utilized to enhance ability of myeloid suppressor cells to kill NK cells.

In some embodiments patient immune cells conditioned by mesenchymal stem cells are utilized to enhance angiogenic activity of myeloid derived suppressor cells [109]. Stimulation of angiogenesis may be utilized to enhance engraftment of allogeneic pancreatic transplants. In some embodiments the ability of myeloid derived suppressor cells to deplete cystine and/or cysteine [110], is augmented by exposure to patient lymphocytes that have been conditioned by regenerative cells.

In one embodiment, the invention provides for a method of making an autologous immunological composition for the treatment of diabetes in humans, comprising: providing a peripheral blood composition from a human patient in need of treatment, extracting CD3.sup.+ T cells, in which the CD3.sup.+ T cells are enriched for T cells reactive to antigens uniquely expressed by the pancreatic islets and subsequently inducing said CD3 T cells to possess a tolerogenic and/or regenerative phenotype through incubation with a mesenchymal stem cell population. In some embodiments, the antigens found on pancreatic islet cells are proteins or peptides. These antigens may be identified by whole exome sequencing and RNAseq of pancreatic, beta cell, and alpha cell tissues the same individual, and HLA binding algorithms applied to determine which pancreatic beta cell specific peptides bind HLA molecules.

In some embodiments, the recipient has been immunized against pancreatic beta cell specific antigens prior to extraction of T cells for reprogramming with mesenchymal stem cells. The practice of inducing a tolerogenic vaccine to diabetes has been previously described. In one embodiment preimmunization with pancreatic associated peptides can be performed as described by others and incorporated by reference [111-141]. In some cases, immunization with GAD peptide may be performed intralymphatically in order to induce a more tolerogenic response [142, 143]. Enhancement of tolerogenicity may be achieved by administration of various supplements such as vitamin D [144], or cytokine blocking agents [145]. Because vaccination with diabetic autoantigens has been previously shown to induce T regulatory cells [146], in some embodiments of the invention, T regulatory cells are assessed in response to vaccination and if needed, additional vaccines and/or tolerogenic interventions may be performed before extract patient cells for tolerogenic reprogramming.

In some embodiments, the immunization consists of intramuscular injection of antigen emulsified in an adjuvant, DNA vaccination plus electroporation, etc.

In one embodiment of the invention, administration of PGE-2, or agents or cells that induce expression of PGE2 is performed in order to increase activity and/or numbers of myeloid derived suppressor cells. Means of administration of PGE-2 can be borrowed from publications which describe induction of myeloid suppressor cells by tumor associated PGE-2 [147]. In another embodiment VEGF is administered to induce augmentation of activity and/or number of myeloid derived suppressor cells [148]. In another embodiment low dose interleukin-2 is administered as a means of augmenting myeloid derived suppressor cell number and/or activity [149].

In one embodiment immunization means are utilized before extraction of patient immune cells for reprogramming by regenerative cells. The patient's T cells, which possess CD3+ are made toleroenic and reactive to one or more pancreatic antigens.

In some embodiments of the invention, prior to expansion, a source of T cells is obtained from a subject. The term “subject” is intended to include living organisms in which an immune response can be elicited (e.g., mammals). Examples of subjects include humans, dogs, cats, mice, rats, and transgenic species thereof. In some embodiments, the subject is a partially or fully HLA-matched healthy donor (i.e., non-cancerous donor). T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as ficoll separation. In one embodiment of the invention, the cells are washed with phosphate buffered saline (PBS). In an alternative embodiment, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media. In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3.sup.+, CD28.sup.+, CD4.sup.+, CD8.sup.+, CD45RA.sup.+, and CD45RO.sup.+ T cells, can be further isolated by positive or negative selection techniques. For example, in some embodiment, T cells are isolated by incubation with anti-CD3/anti-CD28 (i.e., 3.times.28)-conjugated beads, such as DYNABEADS™, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells. Further, use of longer incubation times can increase the efficiency of capture of T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

In one embodiment, enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, or from samples where there are many tumor cells present (i.e., leukemic blood, tumor tissue, etc.). Such populations of cells may have therapeutic value and would be desirable to obtain. In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. In one embodiment, the concentration of cells used is 5.times.10.sup.6/ml. In other embodiments, the concentration used can be from about 1.times.10.sup.5/ml to 1.times.10.sup.6/ml, and any integer value in between.

In other embodiments, the cells may be incubated on a rotator for varying lengths of time at varying speeds at either 2-10.degree. C. or at room temperature. If desired or necessary, T cell populations (i.e., CD3.sup.+ cells) may be depleted from blood preparations prior to ex vivo expansion by a variety of methodologies, including anti-CD3 coated beads or columns, or utilization of the phagocytotic activity of these cells to facilitate removal, or by the use of counterflow centrifugal elutriation. Accordingly, in one embodiment, the invention uses paramagnetic particles of a size sufficient to be engulfed by phagocytotic monocytes. In certain embodiments, the paramagnetic particles are commercially available beads, for example, those produced by Dynal AS under the trade name Dynabeads™. Exemplary Dynabeads™ in this regard are M-280, M-450, and M-500. In one aspect, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be expanded. In certain embodiments the irrelevant beads include beads coated with sheep anti-mouse antibodies, goat anti-mouse antibodies, and human serum albumin. In brief, such depletion of monocytes is performed by preincubating PBMC isolated from whole blood or apheresed peripheral blood with one or more varieties of irrelevant or non-antibody coupled paramagnetic particles at any amount that allows for removal of monocytes (approximately a 20:1 bead:cell ratio) for about 30 minutes to 2 hours at 22 to 37.degree. C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL™ Magnetic Particle Concentrator (DYNAL MPC™)). Assurance of requisite depletion can be monitored by a variety of methodologies known to those of ordinary skill in the art, including flow cytometric analysis of CD14 positive cells, before and after said depletion. T cells for stimulation can also be frozen after the washing step, which does not require the monocyte-removal step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80.degree. C. at a rate of 1.degree. per minute and stored in the vapor phase of a liquid nitrogen storage tank. Other methods of controlled freezing may be used as well as uncontrolled freezing immediately at −20.degree. C. or in liquid nitrogen. The culture of T cells may be performed in the presence of regenerative cells. In some embodiments said regenerative cells are pulsed or primed with an immune stimulatory agent. This is to enhance the ability of the regenerative cells to program T cells, or PBMC. In one ideal embodiment patient PMBC are extracted, incubated with regenerative cells and subsequently administered back to the patient. In other embodiments immune cells from the patient are cultured in the conditioned media of regenerative cells. In some embodiments cells are cultured under hypoxia.

In certain embodiments, cryopreserved cells are thawed and washed as described herein and allowed to rest for one hour at room temperature prior to activation using the methods of the present invention.

In some embodiments, the lymphocytes are taken from a partially or fully HLA-matched, non-cancerous donor. T cells are activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and 7,572,631 with the exception that they are cultured together with regenerative cells.

Infusion of the immune cell population of the present invention enhances, potentiates, or increases the tolerogenic capacity of the immune response, as well as evokes regenerative potential. Generally, the immune response can include the humoral immune response, the cell-mediated immune response, or both. For example, antigen presentation through an immunological pathway involving MHC II proteins or direct B-cell stimulation can produce a humoral response; and, antigens presented through a pathway involving MHC I proteins can elicit the cellular arm of the immune system. A humoral response can be determined by a standard immunoassay for antibody levels in a serum sample from the subject receiving the pharmaceutically acceptable composition. A cellular immune response is a response that involves T cells and can be determined in vitro or in vivo. For example, a general cellular immune response can be determined as the T cell proliferative activity in cells (e.g., peripheral blood leukocytes (PBLs)) sampled from the subject at a suitable time following the administering of a pharmaceutically acceptable composition. Following incubation of e.g., PBMCs with a stimulator for an appropriate period, [.sup.3H]thymidine incorporation can be determined. The subset of T cells that is proliferating can be determined using flow cytometry. T cell cytotoxicity (CTh) can also be determined.

The pharmaceutically acceptable composition can be administered in a therapeutically or a prophylactically effective amount, wherein the pharmaceutically acceptable composition comprises the lymphocyte population of T cells are enriched for T cells reactive to neo-antigens in the recipient and depleted of T cells reactive to antigens on non-cancerous tissues of the recipient, either alone or in combination with one or more other antigens. Administering the pharmaceutically acceptable composition of the present invention to the subject can be carried out using known procedures, and at dosages and for periods of time sufficient to achieve a desired effect. For example, a therapeutically or prophylactically effective amount of the pharmaceutically acceptable composition, can vary according to factors such as the age, sex, and weight of the subject. Dosage regima can be adjusted by one of ordinary skill in the art to elicit the desired immune response including immune responses that provide therapeutic or prophylactic effects.

Administering can be properly timed by the care giver (e.g., physician, veterinarian), and can depend on the clinical condition of the subject, the objectives of administering, and/or other therapies also being contemplated or administered. In some embodiments, an initial dose can be administered, and the subject monitored for either an immunological or clinical response, preferably both. Suitable means of immunological monitoring include using patient's peripheral blood lymphocyte (PBL) as responders and neoplastic cells as stimulators. An immunological reaction also can be determined by a delayed inflammatory response at the site of administering. One or more doses subsequent to the initial dose can be given as appropriate, typically on a monthly, semimonthly, or preferably a weekly basis, until the desired effect is achieved. Thereafter, additional booster or maintenance doses can be given as required, particularly when the immunological or clinical benefit appears to subside.

The lymphocyte compositions of the present invention may be administered either alone, or as a pharmaceutical composition in combination with diluents and/or with other components such as IL-2 or other cytokines or cell populations. Briefly, pharmaceutical lymphocyte compositions of the present invention may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In some embodiments of the invention, In certain embodiments, the present invention provides a method of enhancing activity of myeloid derived suppressor cells by exposing said cells to patient liyphocytes that have been conditioned with regenerative cells. In one embodiment, myeloid derived suppressor cells are generated by a means comprising of: a) contacting pluripotent stem cell with an effective amount of kit ligand (KL) (stem cell factor), vascular endothelial growth factor (VEGF), FMS-like tyrosine kinase 3 (Flt3L), thrombopoietin (TPO), and macrophage colony-stimulating factor (M-CSF); and b) culturing said iPSC cells under conditions suitable for propagation of said cell, thereby obtaining a preparation of an isolated MDSC. In certain embodiments, the method further comprises cryopreservation of said MDSC. In yet additional embodiments, the iPSC cell is a mammalian cell. In certain embodiments, the iPSC cell is a human cell. In yet additional embodiments, the isolated MDSC expresses at least one of the cell surface markers selected from the group consisting of CD33, CD115, F4/80, Ly-6C, CD11b, Gr-1, VEGF receptor, CD40 and IL-4R. Other means of generating MDSC are disclosed such as a) contacting a hematopoietic stem cell (HSC) with an effective amount of kit ligand (KL) (stem cell factor), vascular endothelial growth factor (VEGF), FMS-like tyrosine kinase 3 (F1t3L), thrombopoietin (TPO), and macrophage colony-stimulating factor (M-CSF); and b) culturing said HSC under conditions suitable for propagation of said cell, thereby obtaining a preparation of an isolated MDSC. In certain embodiments, the method further comprises cryopreservation of said MDSC. In yet additional embodiments, the HSC is a mammalian HSC. In yet additional embodiments, the HSC is a human HSC. In yet additional embodiments, the isolated MDSC expresses at least one of the cell surface markers selected from the group consisting of CD33, CD115, VEGF receptor, F4/80, Ly-6C, CD11b, Gr-1, CD40 and IL-4R. In other embodiments, the isolated MDSC derived from a human ES cell or human HSC expresses at least one of the cell surface markers selected from the group consisting of CD11b, CD33, CD15, and CD16. In yet other embodiments, the isolated MDSC expresses CD11b and CD33. In still other embodiments, the isolated MDSC expresses CD11b and Gr-1. In yet additional embodiments, the invention provides an isolated MDSC obtained by any of the methods described herein.

The invention, in some embodiments, teaches the application of Immunological tolerance to the condition of alloantigen reactivity and autoimmunity. In one embodiment the invention teaches the treatment of diabetes. In one embodiment the invention teaches the treatment and/or reversion of type 1 diabetes. It is known that a cardinal feature of the immune system, is allowing for recognition and elimination of pathological threats, while selectively ignoring antigens that belong to the body. Traditionally, autoimmune conditions such as type 1 diabetes or conditions associated with cytokine storm, or allograft rejection are treated with non-specific inhibitors of inflammation such as steroids, as well as immune suppressive agents such as cyclosporine, 5-azathrioprine, and methotrexate. These approaches globally suppress immune functions and have numerous undesirable side effects. Unfortunately, given the substantial decrease in quality of life observed in patients with autoimmunity, the potential of alleviation of autoimmune symptoms outweighs the side effects such as opportunistic infections and increased predisposition to neoplasia.

The invention provides novel stem cell types, methods of manufacture, and therapeutic uses. Provided are means of deriving stem cells possessing regenerative, immune modulatory, anti-inflammatory, and angiogenic/neurogenic activity from umbilical cord tissue such as Wharton's Jelly. In some embodiments manipulation of stem cell “potency” is disclosed through hypoxic manipulation, growth on non-xenogeneic conditions, as well as addition of epigenetic modulators.

The cells of the invention are cultured under hypoxia, in one embodiment, cultured in order to induce and/or augment expression of chemokine receptors. One such receptor is CXCR-4. The population of cells, including population of umbilical cord mesenchymal cells, may be enriched for CXCR-4, such as (or such as about) 50%, 55%, 60%, 65%, 70%, 75%, 80%, 85%, 90%, 95%, 96%, 97%, 98%, 99%, or more of the population expressing CXCR-4, CD31, CD34, or any combination thereof. In addition or alternatively, <1%, <2%, <3%, <4%, <5%, <6%, <7%, <8%, <9%, or <10% of the population of cells may express CD14 and/or CD45. The umbilical cord cells of the invention may further possess markers selected from the group consisting of STRO-1, CD105, CD54, CD56, CD106, HLA-I markers, vimentin, ASMA, collagen-1, fibronectin, LFA-3, ICAM-1, PECAM-1, P-selectin, L-selectin, CD49b/CD29, CD49c/CD29, CD49d/CD29, CD61, CD18, CD29, thrombomodulin, telomerase, CD10, CD13, STRO-2, VCAM-1, CD146, and THY-1, and a combination thereof. In some embodiments said placental cells of the invention are admixed with endothelial cells. Said endothelial cells may express one or more markers selected from the group consisting of: a) extracellular vimentin; b) CD133; c) c-kit; d) VEGF receptor; e) activated protein C receptor; and f) a combination thereof. In some embodiments, the population of endothelial cells comprises endothelial progenitor cells.

The population of cells may be allogeneic, autologous, or xenogenic to an individual, including an individual being administered the population of cells. In some embodiments, the population of cells are matched by mixed lymphocyte reaction matching.

In some embodiments, the population of cells is derived from tissue selected from the group consisting of the placental body, placenta, umbilical cord tissue, peripheral blood, hair follicle, cord blood, Wharton's Jelly, menstrual blood, endometrium, skin, omentum, amniotic fluid, and a combination thereof. In some embodiments, the population of cells, the population of umbilical mesenchymal stem cells, or the population of endothelial cells comprises human umbilical cord derived adherent cells. The human umbilical cord derived adherent cells may express a cytokines selected from the group consisting of) FGF-1; b) FGF-2; c) HGF; d) interleukin-1 receptor antagonist; and e) a combination thereof. In some embodiments, the population of cells, the population of umbilical cord cells express arginase, indoleamine 2,3 deoxygenase, interleukin-10, and/or interleukin 35. In some embodiments, the population of cells, the population of umbilical cord cells, or the population of endothelial cells express hTERT and Oct-4 but does not express a STRO-1 marker.

In some embodiments, the population of cells, the population of umbilical cord cells has an ability to undergo cell division in less than 36 hours in a growth medium. In some embodiments, the population of cells, the population of umbilical cord cells has an ability to proliferate at a rate of 0.9-1.2 doublings per 36 hours in growth media. In some embodiments, the population of cells, the population of umbilical cord cells has an ability to proliferate at a rate of 0.9, 1.0, 1.1, or 1.2 doublings per 36 hours in growth media. The population of cells, population of umbilical cord cells may produce exosomes capable of inducing more than 50% proliferation when the exosomes are cultured with human umbilical cord endothelial cells. The induction of proliferation may occur when the exosomes are cultured with the human umbilical cord endothelial cells at a concentration of 10, 20, 30, 40, 50, 60, 70, 80, 90, 100, 110, 120, 130, 140, 150, 160, 170, 180, 190, 200, or more exosomes per cell.

In some embodiments, a population of cells, including a population of umbilical cells alone, are administered to an individual, including an individual having and acute or chronic pathology, wherein the population of cells may be administered via any suitable route, including as non-limiting examples, intramuscularly and/or intravenously.

In some embodiments, a population of umbilical cord cells is optionally obtained, the population is then optionally contacted via culturing with a population of progenitor for T regulatory cells, wherein the culturing conditions allow for the generation of T regulatory cells, then the generated T regulatory cells are administered to an individual.

In another embodiment of the invention, biologically useful immune cells are generated after culture with regenerative cells, and/or stem cells are disclosed, of the mesenchymal or related lineages, which are therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues. For use in the current invention, the practitioner is thought that ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G0/G1 phase of the cell cycle. In mice and rats these gonocytes resume division within a few days after birth to generate spermatogonial stem cells and eventually undergo differentiation and meiosis related to spermatogenesis. It is known that embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage. Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory. During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress. In one teaching, or embodiment, of the invention, therapeutically reprogrammed cells, in some embodiments mesenchymal stem cells, are provided. Therapeutic reprogramming refers to a maturation process wherein a stem cell is exposed to stimulatory factors according the teachings of the present invention to yield enhanced therapeutic activity. In some embodiments, enhancement of therapeutic activity may be increase proliferation, in other embodiments, it may be enhanced chemotaxis. Other therapeutic characteristics include ability to under resistance to apoptosis, ability to overcome senescence, ability to differentiate into a variety of different cell types effectively, and ability to secrete therapeutic growth factors which enhance viability/activity, of endogenous stem cells. In order to induce therapeutic reprogramming of cells, in some cases, as disclosed herein, of wharton's jelly originating cells, the invention teaches the utilization of stimulatory factors, including without limitation, chemicals, biochemicals and cellular extracts to change the epigenetic programming of cells. These stimulatory factors induce, among other results, genomic methylation changes in the donor DNA. Embodiments of the present invention include methods for preparing cellular extracts from whole cells, cytoplasts, and karyplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation. In another embodiment of the present invention, hybrid stem cells are provided which can be used for cellular regenerative/reparative therapy. The hybrid stem cells of the present invention are pluripotent and customized for the intended recipient so that they are immunologically compatible with the recipient. Hybrid stem cells are a fusion product between a donor cell, or nucleus thereof, and a host cell. Typically the fusion occurs between a donor nucleus and an enucleated host cell. The donor cell can be any diploid cell, including but not limited to, cells from pre-embryos, embryos, fetuses and post-natal organisms. More specifically, the donor cell can be a primordial sex cell, including but not limited to, oogonium or differentiated or undifferentiated spermatogonium, or an embryonic stem cell. Other non-limiting examples of donor cells are therapeutically reprogrammed cells, embryonic stem cells, fetal stem cells and multipotent adult progenitor cells. Preferably the donor cell has the phenotype of the intended recipient. The host cell can be isolated from tissues including, but not limited to, pre-embryos, embryos, fetuses and post-natal organisms and more specifically can include, but is not limited to, embryonic stem cells, fetal stem cells, multipotent adult progenitor cells and adipose-derived stem cells. In a non-limiting example, cultured cell lines can be used as donor cells. The donor and host cells can be from the same individual or different individuals. In one embodiment of the present invention, lymphocytes are used as donor cells and a two-step method is used to purify the donor cells. After the tissues was disassociated, an adhesion step was performed to remove any possible contaminating adherent cells followed by a density gradient purification step. The majority of lymphocytes are quiescent (in G0 phase) and therefore can have a methylation status than conveys greater plasticity for reprogramming. Multipotent or pluripotent stem cells or cell lines useful as donor cells in embodiments of the present invention are functionally defined as stem cells by their ability to undergo differentiation into a variety of cell types including, but not limited to, adipogenic, neurogenic, osteogenic, chondrogenic and cardiogenic cell.

In some embodiments, host cell enucleation for the generation of hybrid stem cells according to the teachings of the present invention can be conducted using a variety of means. In a non-limiting example, ADSCs were plated onto fibronectin coated tissue culture slides and treated with cells with either cytochalasin D or cytochalasin B. After treatment, the cells can be trypsinized, re-plated and are viable for about 72 hours post enucleation. Host cells and donor nuclei can be fused using one of a number of fusion methods known to those of skill in the art, including but not limited to electrofusion, microinjection, chemical fusion or virus-based fusion, and all methods of cellular fusion are envisioned as being within the scope of the present invention. The hybrid stem cells made according to the teachings of the present invention possess surface antigens and receptors from the enucleated host cell but has a nucleus from a developmentally younger cell. Consequently, the hybrid stem cells of the present invention will be receptive to cytokines, chemokines and other cell signaling agents, yet possess a nucleus free from age-related DNA damage. The therapeutically reprogrammed cells and hybrid stem cells made in accordance with the teachings of the present invention are useful in a wide range of therapeutic applications for cellular regenerative/reparative therapy. For example, and not intended as a limitation, the therapeutically reprogrammed cells and hybrid stem cells of the present invention can be used to replenish stem cells in animals whose natural stem cells have been depleted due to age or ablation therapy such as cancer radiotherapy and chemotherapy. In another non-limiting example, the therapeutically reprogrammed cells and hybrid stem cells of the present invention are useful in organ regeneration and tissue repair. In one embodiment of the present invention, therapeutically reprogrammed cells and hybrid stem cells can be used to reinvigorate damaged muscle tissue including dystrophic muscles and muscles damaged by ischemic events such as myocardial infarcts. In another embodiment of the present invention, the therapeutically reprogrammed cells and hybrid stem cells disclosed herein can be used to ameliorate scarring in animals, including humans, following a traumatic injury or surgery. In this embodiment, the therapeutically reprogrammed cells and hybrid stem cells of the present invention are administered systemically, such as intravenously, and migrate to the site of the freshly traumatized tissue recruited by circulating cytokines secreted by the damaged cells. In another embodiment of the present invention, the therapeutically reprogrammed cells and hybrid stem cells can be administered locally to a treatment site in need or repair or regeneration.

In one embodiment, umbilical cord samples were obtained following the delivery of normal term babies with Institutional Review Board approval. A portion of the umbilical cord was then cut into approximately 3 cm long segments. The segments were then placed immediately into 25 ml of phosphate buffered saline without calcium and magnesium (PBS) and 1.times. antibiotics (100 U/ml penicillin, 100 ug/ml streptomycin, 0.025 ug/ml amphotericin B). The tubes were then brought to the lab for dissection within 6 hours. Each 3 cm umbilical cord segment was dissected longitudinally utilizing aseptic technique. The tissue was carefully undermined and the umbilical vein and both umbilical arteries were removed. The remaining segment was sutured inside out and incubated in 25 ml of PBS, 1.times. antibiotic, and 1 mg/ml of collagenase at room temperature. After 16-18 hours the remaining suture and connective tissue was removed and discarded. The cell suspension was separated equally into two tubes, the cells were washed 3.times. by diluting with PBS to yield a final volume of 50 ml per tube, and then centrifuged. Red blood cells were then lysed using a hypotonic solution. Cells were plated onto 6-well plates at a concentration of 5-20.times.10. sup.6 cells per well. UC-MSC were cultured in low-glucose DMEM (Gibco) with 10% FBS (Hyclone), 2 mM L-Glutamine (Gibco), 100 U/ml penicillin, 100 ug/ml streptomycin, 0.025 ug/ml amphotericin B (Gibco). Cells were washed 48 hours after the initial plating with PBS and given fresh media. Cell culture media were subsequently changed twice a week through half media changes. After 7 days or approximately 70-80% confluence, cells were passed using HyQTase (Hyclone) into a 10 cm plate. Cells were then regularly passed 1:2 every 7 days or upon reaching 80% confluence. Alternatively, 0.25% HQ trypsin/EDTA (Hyclone) was used to passage cells in a similar manner.

In one embodiment, tolerogenic dendritic cells may be pulsed with diabetogenic antigens in order to induce an immune response that is tolerogenic, which is subsequently amplified by myeloid derived suppressor cells, and/or ImmCelz. Generation of tolerogenic dendritic cells may be accomplished by manipulating existing protocols for generation of dendritic cells by adding a maturation inhibition step. Protocols used for generating dendritic cells have been described in the literature and are incorporated by reference in melanoma [150-201], soft tissue sarcoma [202], thyroid [203-205], glioma [206-227], multiple myeloma, [228-236], lymphoma [237-239], leukemia [240-247], as well as liver [248-253], lung [254-267], ovarian [268-271], and pancreatic cancer [272-274].

In some embodiments of the invention, administration of cells of the invention is performed for suppression of an inflammatory and/or autoimmune disease. In these situations, it may be necessary to utilize an immune suppressive/or therapeutic adjuvant. Immune suppressants are known in the art and can be selected from a group comprising of: cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-α inhibitors, TNF-α sequestration agents, and methotrexate. More specifically, anti-inflammatory agents may comprise one or more of, e.g., anti-TNF-α, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloropredni sone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, mepredni sone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicyl sulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric.acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid.

In some embodiment of the invention monocytic progenitors are incubated with exosomes derived from regenerative cells. Some embodiments of the methods and compositions provided herein relate to exosomes derived from said regenerative cells. An exosome is a lipid bilayer vesicle that is exocytosed from a cell. The vesicles are of endosomal origin, and range in size between 30-200 nm, including sizes (e.g., diameter) of about 40-100 nm (including about 40 to about 50 nm, about 50 to about 60 nm, about 60 to about 70 nm, about 70 to about 80 nm, about 80 to about 90 nm, about 90 to about 100 nm, and any size therebetween, including endpoints), and, in several embodiments, possess a cup-shaped morphology as revealed by electron microscopy. Depending on the embodiment, exosomes are optionally enriched in a variety of biological factors, including cytokines, growth factors, transcription factors, lipids, and coding and non-coding nucleic acids. Exosomes are found in blood, urine, amniotic fluid, interstitial and extracellular spaces.

In some embodiments, exosomes are isolated by, for example, differential ultracentrifugation, to separate the exosomes from the supernatants of cultured cells. This approach allows for separation of exosomes from nonmembranous particles by exploiting their relatively low buoyant density. Size exclusion allows for their separation from biochemically similar, but biophysically different microvesicles that possess, for example, larger diameters of up to 1,000 nm. Differences in flotation velocity further allows for separation of differentially sized exosomes. In some embodiments, exosome sizes possess a diameter ranging from 30-200 nm, including sizes of about 40-100 nm (including about 40 to about 50 nm, about 50 to about 60 nm, about 60 to about 70 nm, about 70 to about 80 nm, about 80 to about 90 nm, about 90 to about 100 nm, and any size therebetween, including endpoints). In some embodiments, purification relies on specific properties of exosomes of interest. In some embodiments, this includes use of immunoadsorption with a protein of interest to select specific vesicles with exoplasmic or outward orientations.

It is known in the art that differential ultracentrifugation utilizes increasing centrifugal forces (e.g., from 2000.times.g to 10,000.times.g) to separate medium- and larger-sized particles and cell debris from an exosome pellet at 100,000.times.g. In some embodiments, enhanced specificity of exosome purification deploys sequential centrifugation in combination with ultrafiltration, or equilibrium density gradient centrifugation in a sucrose density gradient, to provide for the greater purity of the exosome preparation (flotation density 1.1-1.2 g/ml) or application of a discrete sugar cushion in preparation.

In some embodiments of the invention, ultrafiltration is used to purify exosomes without compromising their biological activity. In some embodiments, membranes with different pore sizes—such as 100 kDa molecular weight cut-off (MWCO) and gel filtration to eliminate smaller particles—are used to avoid the use of a nonneutral pH or non-physiological salt concentration. Currently available tangential flow filtration (TFF) systems are scalable (to >10,000 L), allowing one to not only purify, but concentrate the exosome fractions, and such approaches are less time consuming than differential centrifugation. In some embodiments, HPLC is also used to purify exosomes to homogeneously sized particles and preserve their biological activity as the preparation is maintained at a physiological pH and salt concentration.

Other chemical means can be utilized to isolate exosomes by utilizing differential solubility of exosomes for precipitation techniques, such as addition to volume-excluding polymers (for example, polyethylene glycols (PEGs)), possibly combined additional rounds of centrifugation or filtration. For example, a precipitation reagent, ExoQuick®, are added to conditioned cell media to quickly and rapidly precipitate a population of exosomes, although re-suspension of pellets prepared via this technique can be difficult. Flow field-flow fractionation (FlFFF) is an elution-based technique that is used to separate and characterize macromolecules (for example, proteins) and nano- to micro-sized particles (for example, organelles and cells). In some embodiments, FlFFF is applied to fractionate exosomes from culture media.

Alternative/supplemental methodological techniques may be applied to isolated specific exosomes of interest, such as relying on antibody immunoaffinity to recognizing certain exosome-associated antigens. In some embodiments, exosomes express extracellular domains of membrane-bound receptors at the surface of their membranes. In some embodiments, this surface expression profile is used for isolating and segregating exosomes in connection with their parental cellular origin, based on a shared antigenic profile. Conjugation to magnetic beads, chromatography matrices, plates or microfluidic devices allows isolating of specific exosome populations of interest. In some embodiments, the specific exosome population of interest is related to its production from a parent cell of interest or associated cellular regulatory state. Other affinity-capture methods use lectins which bind to specific saccharide residues on the exosome surface.

Some embodiments of the methods and compositions provided herein relate to a composition that includes a plurality of exosomes derived from said regenerative cells. In some embodiments, the plurality of exosomes is isolated from regenerative cells are grown in serum-free media, and may include exosomes with a diameter of about 90 nm to about 200 nm and are CD81+, CD63+, or both, and further wherein administration of the composition an immunomodulatory/regenerative property or properties to said monocytic cells

In some embodiments, the plurality of exosomes is generated by a method including providing a population of cells, and isolating a plurality of exosomes from the population of cells. In some embodiments, the regenerative cells are stem cells, progenitors or precursors. Mixtures of such cell types is used, according to several embodiments. In some embodiments, the stem cells, progenitors or precursors are associated with a specific tissue whose healing is desired In some embodiments, the stem cells, progenitors or precursors are pluripotent stem cells (pSCs), such as embryonic stem cells (ESCs) and induced pluripotent stem cells (iPSCs) derived from any one of various somatic sources in the body such as fibroblasts, blood and hematopoietic stem cells (hSCs), immune cells, bone and bone marrow, neural tissue, among others. In some embodiments, the stem cells, progenitors or precursors include hSCs, mesenchymal stem cells (MSCs), and/or endothelial precursor cells (EPCs). In some embodiments, the cells are stem cells, progenitors and/or precursors derived from human biopsy tissue. In some embodiments, the cells are stem cells, progenitors or precursors are a primary culture. In some embodiments, the cells are stem cells, progenitors or precursors are a cell line capable of serial passaging. In some embodiments, the exosomes are synthetic.

In some embodiments, the microRNAs include one or more of miR-146a, miR22, miR-24, miR-210, miR-150, miR-140-3p, miR-19a, miR-27b, miR-19b, miR-27a, miR-376c, miR-128, miR-320a, miR-143, miR-21, miR-130a, miR-9, miR-185, and miR-23a. In some embodiments, the plurality of exosomes includes one or more exosomes enriched in at least one of miR-146a, miR-22, or miR-24.

In some embodiments of the invention, stimulation of myeloid cells possessing suppressive properties is accomplished in vivo. In some embodiments small number of monocytic cells generating IL-10 are administered in a patient together with low-dose interleukin-2. In some embodiments of the invention, stimulation of T regulatory cells in vivo is accomplished by administration of Aldesleukin (Proleukin, Novartis), which is a commercially available IL-2 licensed for the treatment of metastatic renal cell carcinoma in the UK. It is produced by recombinant DNA technology using an Escherichia coli strain, which contains a genetically engineered modification of the human IL-2 gene, and is administered either intravenously or subcutaneously (SC). Following short intervenous infusion, its pharmacokinetic profile is typified by high plasma concentrations, rapid distribution into the extravascular space and a rapid renal clearance. The recommended doses for continuous infusion and subcutaneous injection (as detailed in the Summary of Product Characteristics) are repeated cycles of 18×10⁶ IU per m² per 24 hours for 5 days and repeated doses of 18×10⁶ IU, respectively. Peak plasma levels are reached in 2-6 hours after SC administration, with bioavailability of aldesleukin ranging between 31% and 47%. The process of absorption and elimination of subcutaneous aldesleukin is described by a one-compartment model, with a 45 min absorption half-life and an elimination half-life of 3-5 hours

Natural IL-2 was first identified in 1976 as a growth factor for T lymphocytes. It is produced by human cluster designation (CD) 4+ and some CD8+ T-cells and is synthesized mainly by activated T-cells, in particular CD4.sup.+ helper T cells. It stimulates the proliferation and differentiation of T cells, induces the generation of cytotoxic T lymphocytes (CTLs) and the differentiation of peripheral blood lymphocytes to cytotoxic cells and lymphokine-activated killer (LAK) cells, promotes cytokine and cytolytic molecule expression by T cells, facilities the proliferation and differentiation of B-cells and the synthesis of immunoglobulin by B-cells, and stimulates the generation, proliferation and activation of natural killer (NK). IL-2 is known to play a central role in the generation of immune responses. In cancer clinical trials, high-dose recombinant IL-2 (e.g., IV bolus dose of 600,000 international units (IU)/kg every 8 hours for up to 14 doses) demonstrated antitumor activity in metastatic renal cell carcinoma (RCC) and metastatic melanoma. Accordingly, such high-dose IL-2 was approved for the treatment of metastatic RCC in Europe in 1989 and in the US in 1992. In 1998, approval was obtained to treat patients with metastatic melanoma. Recombinant human IL-2 (Aldesleukin) (Proleukin®-Novartis Inc. & Prometheus Labs Inc.) is currently approved by the United States Food and Drug Administration (US FDA). However, IL-2 has a dual function in the immune response in that it not only mediates expansion and activity of effector cells, but also is crucially involved in maintaining peripheral immune tolerance. A major mechanism underlying peripheral self-tolerance is IL-2 induced activation-induced cell death (AICD) in T cells. AICD is a process by which fully activated T cells undergo programmed cell death through engagement of cell surface-expressed death receptors such as CD95 (also known as Fas) or the TNF receptor. When antigen-activated T cells expressing a high-affinity IL-2 receptor (after previous exposure to IL-2) during proliferation are re-stimulated with antigen via the T cell receptor (TCR)/CD 3 complex, the expression of Fas ligand (FasL) and/or tumor necrosis factor (TNF) is induced, making the cells susceptible for Fas-mediated apoptosis. This process is IL-2 dependent and mediated via STATS. By the process of AICD in T lymphocytes tolerance can not only be established to self-antigens, but also to persistent antigens that are clearly not part of the host's makeup, such as tumor antigens.

### REFERENCES

1. Gronvik, K. O., D. W. Hoskin, and R. A. Murgita, Monoclonal antibodies against murine neonatal and pregnancy-associated natural suppressor cells induce resorption of the fetus. Scand J Immunol, 1987. 25(5): p. 533-40.
2. Maier, T., J. H. Holda, and H. N. Claman, Natural suppressor cells. Prog Clin Biol Res, 1989. 288: p. 235-44.
3. Hoskin, D. W., D. A. Bowser, and J. C. Brooks-Kaiser, Soybean agglutinin-positive natural suppressor cells in mouse bone marrow inhibit interleukin 2 production and utilization in mixed lymphocyte reactions. J Leukoc Biol, 1992. 51(6): p. 649-56.
4. Brooks-Kaiser, J. C. and D. W. Hoskin, Inhibition of DNA synthesis and IL-2 bioactivity in MLR by splenic pregnancy-associated natural suppressor cells involves the production of a TGF-beta 1-like molecule and a second distinct inhibitory factor. J Reprod Immunol, 1993. 25(1): p. 31-49.
5. Segerson, E. C. and P. K. Beetham, Suppressor activity of bone marrow cells and localization of fluorescent-labeled bone marrow cells within ovine endometrial tissue. J Anim Sci, 2000. 78(3): p. 709-17.
6. Maier, T., J. H. Holda, and H. N. Claman, Synergism between T and non-T cells in the in vivo induction and in vitro expression of graft-vs.-host disease-induced natural suppressor cells. J Exp Med, 1985. 162(3): p. 979-92.
7. Holda, J. H., T. Maier, and H. N. Claman, Murine graft-versus-host disease across minor barriers: immunosuppressive aspects of natural suppressor cells. Immunol Rev, 1985. 88: p. 87-105.
8. Hertel-Wulff, B., et al., Prevention of graft-versus-host disease by natural suppressor cells. Transplant Proc, 1987. 19(1 Pt 1): p. 536-9.
9. Strober, S., B. Hertel-Wulff, and R. B. Schwadron, Role of natural suppressor cells in bone marrow transplantation. Transplant Proc, 1987. 19(6 Suppl 7): p. 88-94.
10. Sykes, M., A. Eisenthal, and D. H. Sachs, Mechanism of protection from graft-vs-host disease in murine mixed allogeneic chimeras. I. Development of a null cell population suppressive of cell-mediated lympholysis responses and derived from the syngeneic bone marrow component. J Immunol, 1988. 140(9): p. 2903-11.
11. Sykes, M., Y. Sharabi, and D. H. Sachs, Achieving alloengraftment without graft-versus-host disease: approaches using mixed allogeneic bone marrow transplantation. Bone Marrow Transplant, 1988. 3(5): p. 379-86.
12. Sykes, M., Y. Sharabi, and D. H. Sachs, Natural suppressor cells in spleens of irradiated, bone marrow-reconstituted mice and normal bone marrow: lack of Sca-1 expression and enrichment by depletion of Mac1-positive cells. Cell Immunol, 1990. 127(2): p. 260-74.
13. Ramsamooj, R., et al., Mechanisms of alloimmune tolerance associated with mixed chimerism induced by vascularized bone marrow transplants. Cell Transplant, 2002. 11(7): p. 683-93.
14. von Boehmer, H., Peptide-based instruction of suppressor commitment in naive T cells and dynamics of immunosuppression in vivo. Scand J Immunol, 2005. 62 Suppl 1: p. 49-54.
15. Vinuela, J. E., et al., Antigen shedding vs. development of natural suppressor cells as mechanism of tumor escape in mice bearing Ehrlich tumor. Int J Cancer, 1991. 47(1): p. 86-91.
16. Hayamizu, K., et al., Induction of natural suppressor-like cells from human adult peripheral blood lymphocytes by a K562-derived factor. Transplantation, 1993. 55(6): p. 1403-8.
17. Young, M. R., et al., Vitamin D3 treatment of tumor bearers can stimulate immune competence and reduce tumor growth when treatment coincides with a heightened presence of natural suppressor cells. Cancer Lett, 1996. 104(2): p. 153-61.
18. Young, M. R., et al., Increased recurrence and metastasis in patients whose primary head and neck squamous cell carcinomas secreted granulocyte-macrophage colony-stimulating factor and contained CD34+ natural suppressor cells. Int J Cancer, 1997. 74(1): p. 69-74.
19. Forghani, P., M. R. Khorramizadeh, and E. K. Waller, Natural suppressor cells; past, present and future. Front Biosci (Elite Ed), 2012. 4: p. 1237-45.
20. Sharma, N., et al., LILRB4 suppresses immunity in solid tumors and is a potential target for immunotherapy. J Exp Med, 2021. 218(7).
21. Liu, J., et al., LILRB4, from the immune system to the disease target. Am J Transl Res, 2020. 12(7): p. 3149-3166.
22. Cella, M., et al., Plasmacytoid monocytes migrate to inflamed lymph nodes and produce large amounts of type I interferon. Nat Med, 1999. 5(8): p. 919-23.
23. Chang, C. C., et al., Tolerization of dendritic cells by T(S) cells: the crucial role of inhibitory receptors ILT3 and ILT4. Nat Immunol, 2002. 3(3): p. 237-43.
24. Suciu-Foca, N., J. S. Manavalan, and R. Cortesini, Generation and function of antigen-specific suppressor and regulatory T cells. Transpl Immunol, 2003. 11(3-4): p. 235-44.
25. Manavalan, J. S., et al., High expression of ILT3 and ILT4 is a general feature of tolerogenic dendritic cells. Transpl Immunol, 2003. 11(3-4): p. 245-58.
26. Ju, X. S., et al., Immunoglobulin-like transcripts ILT2, ILT3 and ILT7 are expressed by human dendritic cells and down-regulated following activation. Gene, 2004. 331: p. 159-64.
27. Manavalan, J. S., et al., Alloantigen specific CD8+CD28-FOXP3+ Tsuppressor cells induce ILT3+ ILT4+ tolerogenic endothelial cells, inhibiting alloreactivity. Int Immunol, 2004. 16(8): p. 1055-68.
28. LeMaoult, J., et al., HLA-G up-regulates ILT2, ILT3, ILT4, and KIR2DL4 in antigen presenting cells, NK cells, and T cells. FASEB J, 2005. 19(6): p. 662-4.
29. Suciu-Foca, N. and R. Cortesini, Central role of ILT3 in the T suppressor cell cascade. Cell Immunol, 2007. 248(1): p. 59-67.
30. Kasai, S., et al., A novel regulatory role of gp49B on dendritic cells in T-cell priming. Eur J Immunol, 2008. 38(9): p. 2426-37.
31. Vlad, G., R. Cortesini, and N. Suciu-Foca, CD8+ Tsuppressor cells and the ILT3 master switch. Hum Immunol, 2008. 69(11): p. 681-6.
32. Vlad, G., et al., Immunoglobulin-like transcript 3: A crucial regulator of dendritic cell function. Hum Immunol, 2009. 70(5): p. 340-4.
33. Vlad, G., et al., Interleukin-10 induces the upregulation of the inhibitory receptor ILT4 in monocytes from HIV positive individuals. Hum Immunol, 2003. 64(5): p. 483-9.
34. Gleissner, C. A., et al., IL-10 inhibits endothelium-dependent T cell costimulation by up-regulation of ILT3/4 in human vascular endothelial cells. Eur J Immunol, 2007. 37(1): p. 177-92.
35. Buckland, M., et al., Aspirin modified dendritic cells are potent inducers of allo-specific regulatory T-cells. Int Immunopharmacol, 2006. 6(13-14): p. 1895-901.
36. Svajger, U., A. Vidmar, and M. Jeras, Niflumic acid renders dendritic cells tolerogenic and up-regulates inhibitory molecules ILT3 and ILT4. Int Immunopharmacol, 2008. 8(7): p. 997-1005.
37. Graca, L., B. Silva-Santos, and A. Coutinho, The blind-spot of regulatory T cells. Eur J Immunol, 2006. 36(4): p. 802-5.
38. Lehner, T., Special regulatory T cell review: The resurgence of the concept of contrasuppression in immunoregulation. Immunology, 2008. 123(1): p. 40-4.
39. Cools, N., et al., Regulatory T cells and human disease. Clin Dev Immunol, 2007. 2007: p. 89195.
40. Yamazaki, S. and R. M. Steinman, Dendritic cells as controllers of antigen-specific Foxp3+ regulatory T cells. J Dermatol Sci, 2009. 54(2): p. 69-75.
41. Curotto de Lafaille, M. A. and J. J. Lafaille, Natural and adaptive foxp3+ regulatory T cells: more of the same or a division of labor? Immunity, 2009. 30(5): p. 626-35.
42. Shalev, I., et al., Making sense of regulatory T cell suppressive function. Semin Immunol, 2011. 23(4): p. 282-92.
43. Kushwah, R. and J. Hu, Role of dendritic cells in the induction of regulatory T cells. Cell Biosci, 2011. 1(1): p. 20.
44. Gravano, D. M. and D. A. Vignali, The battle against immunopathology: infectious tolerance mediated by regulatory T cells. Cell Mol Life Sci, 2012. 69(12): p. 1997-2008.
45. Namdev, P., et al., Monocytic-Myeloid Derived Suppressor Cells of HIV-Infected Individuals With Viral Suppression Exhibit Suppressed Innate Immunity to Mycobacterium tuberculosis. Front Immunol, 2021. 12: p. 647019.
46. Nagaraj, S., et al., Mechanism of T cell tolerance induced by myeloid-derived suppressor cells. J Immunol, 2010. 184(6): p. 3106-16.
47. Sinha, P., V. K. Clements, and S. Ostrand-Rosenberg, Reduction of myeloid-derived suppressor cells and induction of M1 macrophages facilitate the rejection of established metastatic disease. J Immunol, 2005. 174(2): p. 636-45.
48. Bunt, S. K., et al., Inflammation induces myeloid-derived suppressor cells that facilitate tumor progression. J Immunol, 2006. 176(1): p. 284-90.
49. Ochoa, A. C., et al., Arginase, prostaglandins, and myeloid-derived suppressor cells in renal cell carcinoma. Clin Cancer Res, 2007. 13(2 Pt 2): p. 721s-726s.
50. Sinha, P., et al., Cross-talk between myeloid-derived suppressor cells and macrophages subverts tumor immunity toward a type 2 response. J Immunol, 2007. 179(2): p. 977-83.
51. Talmadge, J. E., Pathways mediating the expansion and immunosuppressive activity of myeloid-derived suppressor cells and their relevance to cancer therapy. Clin Cancer Res, 2007. 13(18 Pt 1): p. 5243-8.
52. Movahedi, K., et al., Identification of discrete tumor-induced myeloid-derived suppressor cell subpopulations with distinct T cell-suppressive activity. Blood, 2008. 111(8): p. 4233-44.
53. Sawanobori, Y., et al., Chemokine-mediated rapid turnover of myeloid-derived suppressor cells in tumor-bearing mice. Blood, 2008. 111(12): p. 5457-66.
54. Nagaraj, S. and D. I. Gabrilovich, Tumor escape mechanism governed by myeloid-derived suppressor cells. Cancer Res, 2008. 68(8): p. 2561-3.
55. Serafini, P., et al., Myeloid-derived suppressor cells promote cross-tolerance in 8-cell lymphoma by expanding regulatory T cells. Cancer Res, 2008. 68(13): p. 5439-49.
56. Watanabe, S., et al., Tumor-induced CD11b+Gr-1+ myeloid cells suppress T cell sensitization in tumor-draining lymph nodes. J Immunol, 2008. 181(5): p. 3291-300.
57. Youn, J. I., et al., Subsets of myeloid-derived suppressor cells in tumor-bearing mice. J Immunol, 2008. 181(8): p. 5791-802.
58. Rodriguez, P. C., et al., Arginase I-producing myeloid-derived suppressor cells in renal cell carcinoma are a subpopulation of activated granulocytes. Cancer Res, 2009. 69(4): p. 1553-60.
59. Ostrand-Rosenberg, S. and P. Sinha, Myeloid-derived suppressor cells: linking inflammation and cancer. J Immunol, 2009. 182(8): p. 4499-506.
60. Stewart, T. J., et al., Modulating the expression of IFN regulatory factor 8 alters the protumorigenic behavior of CD11b+Gr-1+ myeloid cells. J Immunol, 2009. 183(1): p. 117-28.
61. Ilkovitch, D. and D. M. Lopez, The liver is a site for tumor-induced myeloid-derived suppressor cell accumulation and immunosuppression. Cancer Res, 2009. 69(13): p. 5514-21.
62. Hoechst, B., et al., Myeloid derived suppressor cells inhibit natural killer cells in patients with hepatocellular carcinoma via the NKp30 receptor. Hepatology, 2009. 50(3): p. 799-807.
63. Pan, P. Y., et al., Immune stimulatory receptor CD40 is required for T-cell suppression and T regulatory cell activation mediated by myeloid-derived suppressor cells in cancer. Cancer Res, 2010. 70(1): p. 99-108.
64. Poschke, I., et al., Immature immunosuppressive CD14+HLA-DR-/low cells in melanoma patients are Stat3hi and overexpress CD80, CD83, and DC-sign. Cancer Res, 2010. 70(11): p. 4335-45.
65. Dugast, A. S., et al., Myeloid-derived suppressor cells accumulate in kidney allograft tolerance and specifically suppress effector T cell expansion. J Immunol, 2008. 180(12): p. 7898-906.
66. Zhang, W., et al., Human inhibitory receptor immunoglobulin-like transcript 2 amplifies CD11b+Gr1+ myeloid-derived suppressor cells that promote long-term survival of allografts. Transplantation, 2008. 86(8): p. 1125-34.
67. Dugast, A. S. and B. Vanhove, Immune regulation by non-lymphoid cells in transplantation. Clin Exp Immunol, 2009. 156(1): p. 25-34.
68. De Wilde, V., et al., Endotoxin-induced myeloid-derived suppressor cells inhibit alloimmune responses via heme oxygenase-1. Am J Transplant, 2009. 9(9): p. 2034-47.
69. Natarajan, S. and A. W. Thomson, Tolerogenic dendritic cells and myeloid-derived suppressor cells: potential for regulation and therapy of liver auto-and alloimmunity. Immunobiology, 2010. 215(9-10): p. 698-703.
70. Boros, P., et al., Myeloid-derived suppressor cells: natural regulators for transplant tolerance. Hum Immunol, 2010. 71(11): p. 1061-6.
71. Adeegbe, D., et al., In vivo induction of myeloid suppressor cells and CD4(+)Foxp3(+)T regulatory cells prolongs skin allograft survival in mice. Cell Transplant, 2011. 20(6): p. 941-54.
72. Highfill, S. L., et al., Bone marrow myeloid-derived suppressor cells (MDSCs) inhibit graft-versus-host disease (GVHD) via an arginase-1-dependent mechanism that is up-regulated by interleukin-13. Blood, 2010. 116(25): p. 5738-47.
73. Serafini, P., et al., Phosphodiesterase-5 inhibition augments endogenous antitumor immunity by reducing myeloid-derived suppressor cell function. J Exp Med, 2006. 203(12): p. 2691-702.
74. Nefedova, Y., et al., Mechanism of all-trans retinoic acid effect on tumor-associated myeloid-derived suppressor cells. Cancer Res, 2007. 67(22): p. 11021-8.
75. Melani, C., et al., Amino-biphosphonate-mediated MMP-9 inhibition breaks the tumor-bone marrow axis responsible for myeloid-derived suppressor cell expansion and macrophage infiltration in tumor stroma. Cancer Res, 2007. 67(23): p. 11438-46.
76. Ko, J. S., et al., Sunitinib mediates reversal of myeloid-derived suppressor cell accumulation in renal cell carcinoma patients. Clin Cancer Res, 2009. 15(6): p. 2148-57.
77. Nagaraj, S., et al., Anti-inflammatory triterpenoid blocks immune suppressive function of MDSCs and improves immune response in cancer. Clin Cancer Res, 2010. 16(6): p. 1812-23.
78. Vincent, J., et al., 5-Fluorouracil selectively kills tumor-associated myeloid-derived suppressor cells resulting in enhanced T cell-dependent antitumor immunity. Cancer Res, 2010. 70(8): p. 3052-61.
79. Veltman, J. D., et al., COX-2 inhibition improves immunotherapy and is associated with decreased numbers of myeloid-derived suppressor cells in mesothelioma. Celecoxib influences MDSC function. BMC Cancer, 2010. 10: p. 464.
80. Li, H., et al., Cancer-expanded myeloid-derived suppressor cells induce anergy of NK cells through membrane-bound TGF-beta 1. J Immunol, 2009. 182(1): p. 240-9.
81. Mandruzzato, S., et al., IL4Ralpha+ myeloid-derived suppressor cell expansion in cancer patients. J Immunol, 2009. 182(10): p. 6562-8.
82. Zheng, Z., et al., IL-6 Promotes the Proliferation and Immunosuppressive Function of Myeloid-Derived Suppressor Cells via the MAPK Signaling Pathway in Bladder Cancer. Biomed Res Int, 2021. 2021: p. 5535578.
83. Smith, A. D., et al., Autocrine IL6-Mediated Activation of the STAT3-DNMT Axis Silences the TNFalpha-RIP1 Necroptosis Pathway to Sustain Survival and Accumulation of Myeloid-Derived Suppressor Cells. Cancer Res, 2020. 80(15): p. 3145-3156.
84. Ibrahim, M. L., et al., Expression profiles and function of IL6 in polymorphonuclear myeloid-derived suppressor cells. Cancer Immunol Immunother, 2020. 69(11): p. 2233-2245.
85. Xu, Z., et al., Upregulation of IL-6 in CUL4B-deficient myeloid-derived suppressive cells increases the aggressiveness of cancer cells. Oncogene, 2019. 38(30): p. 5860-5872.
86. Tomic, S., et al., Prostaglanin-E2 Potentiates the Suppressive Functions of Human Mononuclear Myeloid-Derived Suppressor Cells and Increases Their Capacity to Expand IL-10-Producing Regulatory T Cell Subsets. Front Immunol, 2019. 10: p. 475.
87. Cheng, P., et al., Inhibition of dendritic cell differentiation and accumulation of myeloid-derived suppressor cells in cancer is regulated by 5100A9 protein. J Exp Med, 2008. 205(10): p. 2235-49.
88. Xiang, X., et al., Induction of myeloid-derived suppressor cells by tumor exosomes. Int J Cancer, 2009. 124(11): p. 2621-33.
89. Liu, Y., et al., Contribution of MyD88 to the tumor exosome-mediated induction of myeloid derived suppressor cells. Am J Pathol, 2010. 176(5): p. 2490-9.
90. Greifenberg, V., et al., Myeloid-derived suppressor cell activation by combined LPS and IFN-gamma treatment impairs DC development. Eur J Immunol, 2009. 39(10): p. 2865-76.
91. Morales, J. K., et al., GM-CSF is one of the main breast tumor-derived soluble factors involved in the differentiation of CD11b-Gr1-bone marrow progenitor cells into myeloid-derived suppressor cells. Breast Cancer Res Treat, 2010. 123(1): p. 39-49.
92. Dolcetti, L., et al., Hierarchy of immunosuppressive strength among myeloid-derived suppressor cell subsets is determined by GM-CSF. Eur J Immunol, 2010. 40(1): p. 22-35.
93. Lee, J. M., et al., Agonistic Anti-CD137 Monoclonal Antibody Treatment Induces CD11bGr-1 Myeloid-derived Suppressor Cells. Immune Netw, 2010. 10(3): p. 104-8.
94. Priceman, S. J., et al., Targeting distinct tumor-infiltrating myeloid cells by inhibiting CSF-1 receptor: combating tumor evasion of antiangiogenic therapy. Blood, 2010. 115(7): p. 1461-71.
95. Martino, A., et al., Mycobacterium bovis bacillus Calmette-Guerin vaccination mobilizes innate myeloid-derived suppressor cells restraining in vivo T cell priming via IL-1R-dependent nitric oxide production. J Immunol, 2010. 184(4): p. 2038-47.
96. Zhang, H. and G. G. Meadows, Chronic alcohol consumption enhances myeloid-derived suppressor cells in B16BL6 melanoma-bearing mice. Cancer Immunol Immunother, 2010. 59(8): p. 1151-9.
97. Xiang, X., et al., TLR2-mediated expansion of MDSCs is dependent on the source of tumor exosomes. Am J Pathol, 2010. 177(4): p. 1606-10.
98. Sander, L. E., et al., Hepatic acute-phase proteins control innate immune responses during infection by promoting myeloid-derived suppressor cell function. J Exp Med, 2010. 207(7): p. 1453-64.
99. Dardalhon, V., et al., Tim-3/galectin-9 pathway: regulation of Th1 immunity through promotion of CD11b+Ly-6G+ myeloid cells. J Immunol, 2010. 185(3): p. 1383-92.
100. Rodriguez, P. C. and A. C. Ochoa, Arginine regulation by myeloid derived suppressor cells and tolerance in cancer: mechanisms and therapeutic perspectives. Immunol Rev, 2008. 222: p. 180-91.
101. Xu, L., et al., IL-17-producing gammadeltaT cells ameliorate intestinal acute graft-versus-host disease by recruitment of Gr-1(+)CD11b(+) myeloid-derived suppressor cells. Bone Marrow Transplant, 2021.
102. Nicholson, L. B., B. J. Raveney, and M. Munder, Monocyte dependent regulation of autoimmune inflammation. Curr Mol Med, 2009. 9(1): p. 23-9.
103. Carmi, Y., et al., Microenvironment-derived IL-1 and IL-17 interact in the control of lung metastasis. J Immunol, 2011. 186(6): p. 3462-71.
104. Ma, S., et al., IL-17A produced by gammadelta T cells promotes tumor growth in hepatocellular carcinoma. Cancer Res, 2014. 74(7): p. 1969-82.
105. Wu, P., et al., gammadeltaT17 cells promote the accumulation and expansion of myeloid-derived suppressor cells in human colorectal cancer. Immunity, 2014. 40(5): p. 785-800.
106. Yang, Z., et al., Mast cells mobilize myeloid-derived suppressor cells and Treg cells in tumor microenvironment via IL-17 pathway in murine hepatocarcinoma model. PLoS One, 2010. 5(1): p. e8922.
107. He, D., et al., IL-17 promotes tumor development through the induction of tumor promoting microenvironments at tumor sites and myeloid-derived suppressor cells. J Immunol, 2010. 184(5): p. 2281-8.
108. Nausch, N., et al., Mononuclear myeloid-derived “suppressor” cells express RAE-1 and activate natural killer cells. Blood, 2008. 112(10): p. 4080-9.
109. Kujawski, M., et al., Stat3 mediates myeloid cell-dependent tumor angiogenesis in mice. J Clin Invest, 2008. 118(10): p. 3367-77.
110. Srivastava, M. K., et al., Myeloid-derived suppressor cells inhibit T-cell activation by depleting cystine and cysteine. Cancer Res, 2010. 70(1): p. 68-77.
111. Tavira, B., et al., Effect of simultaneous vaccination with H1N1 and GAD-alum on GAD65-induced immune response. Diabetologia, 2017. 60(7): p. 1276-1283.
112. Ludvigsson, J., et al., GAD-treatment of children and adolescents with recent-onset type 1 diabetes preserves residual insulin secretion after 30 months. Diabetes Metab Res Rev, 2014. 30(5): p. 405-14.
113. Krause, S., et al., GAD autoantibody affinity in adult patients with latent autoimmune diabetes, the study participants of a GAD65 vaccination trial. Diabetes Care, 2014. 37(6): p. 1675-80.
114. Ludvigsson, J., et al., Extended evaluation of the safety and efficacy of GAD treatment of children and adolescents with recent-onset type 1 diabetes: a randomised controlled trial. Diabetologia, 2011. 54(3): p. 634-40.
115. Ludvigsson, J., GAD65: a prospective vaccine for treating Type 1 diabetes? Expert Opin Biol Ther, 2017. 17(8): p. 1033-1043.
116. Axelsson, S., et al., Cellular and humoral immune responses in type 1 diabetic patients participating in a phase III GAD-alum intervention trial. Diabetes Care, 2013. 36(11): p. 3418-24.
117. Ludvigsson, J., et al., GAD treatment and insulin secretion in recent-onset type 1 diabetes. N Engl J Med, 2008. 359(18): p. 1909-20.
118. Elding Larsson, H., et al., Safety and efficacy of autoantigen-specific therapy with 2 doses of alum formulated glutamate decarboxylase in children with multiple islet autoantibodies and risk for type 1 diabetes: A randomized clinical trial. Pediatr Diabetes, 2018. 19(3): p. 410-419.
119. Ludvigsson, J., et al., GAD65 antigen therapy in recently diagnosed type 1 diabetes mellitus. N Engl J Med, 2012. 366(5): p. 433-42.
120. Cheramy, M., et al., GAD-alum treatment in patients with type 1 diabetes and the subsequent effect on GADA IgG subclass distribution, GAD65 enzyme activity and humoral response. Clin Immunol, 2010. 137(1): p. 31-40.
121. Lind, A., et al., A/H1N1 antibodies and TRIB2 autoantibodies in narcolepsy patients diagnosed in conjunction with the Pandemrix vaccination campaign in Sweden 2009-2010. J Autoimmun, 2014. 50: p. 99-106.
122. Pihl, M., et al., GAD-specific T cells are induced by GAD-alum treatment in Type-1 diabetes patients. Clin Immunol, 2017. 176: p. 114-121.
123. Axelsson, S., et al., Early induction of GAD(65)-reactive Th2 response in type 1 diabetic children treated with alum formulated GAD(65). Diabetes Metab Res Rev, 2010. 26(7): p. 559-68.
124. Wherrett, D. K., et al., Antigen-based therapy with glutamic acid decarboxylase (GAD) vaccine in patients with recent-onset type 1 diabetes: a randomised double-blind trial. Lancet, 2011. 378(9788): p. 319-27.
125. Skoglund, C., et al., GAD autoantibody epitope pattern after GAD-alum treatment in children and adolescents with type 1 diabetes. Pediatr Diabetes, 2012. 13(3): p. 244-50.
126. Elding Larsson, H., et al., Pandemrix(R) vaccination is not associated with increased risk of islet autoimmunity or type 1 diabetes in the TEDDY study children. Diabetologia, 2018. 61(1): p. 193-202.
127. Agardh, C. D., et al., GAD65 vaccination: 5 years of follow-up in a randomised dose-escalating study in adult-onset autoimmune diabetes. Diabetologia, 2009. 52(7): p. 1363-8.
128. Hjorth, M., et al., GAD-alum treatment induces GAD65-specific CD4+CD25highFOXP3+ cells in type 1 diabetic patients. Clin Immunol, 2011. 138(1): p. 117-26.
129. Axelsson, S., et al., Long-lasting immune responses 4 years after GAD-alum treatment in children with type 1 diabetes. PLoS One, 2011. 6(12): p. e29008.
130. Boettler, T., et al., The clinical and immunological significance of GAD-specific autoantibody and T-cell responses in type 1 diabetes. J Autoimmun, 2013. 44: p. 40-8.
131. Morales, A. E. and K. M. Thrailkill, GAD-alum immunotherapy in Type 1 diabetes mellitus. Immunotherapy, 2011. 3(3): p. 323-32.
132. Bekris, L. M., et al., GAD65 autoantibody epitopes in adult patients with latent autoimmune diabetes following GAD65 vaccination. Diabet Med, 2007. 24(5): p. 521-6.
133. Yeo, L. and M. Peakman, Antigen-specific immunotherapy and influenza vaccination in type 1 diabetes: timing is everything. Diabetologia, 2017. 60(7): p. 1180-1184.
134. Choat, H. M., et al., Effect of gamma aminobutyric acid (GABA) or GABA with glutamic acid decarboxylase (GAD) on the progression of type 1 diabetes mellitus in children: Trial design and methodology. Contemp Clin Trials, 2019. 82: p. 93-100.
135. Ludvigsson, J., Therapy with GAD in diabetes. Diabetes Metab Res Rev, 2009. 25(4): p. 307-15.
136. Larsson, H. E. and A. Lernmark, Does immune-tolerance treatment with Alum formulated GAD65 protect insulin-production in the pancreatic islet beta cells? Hum Vaccin, 2011. 7(1): p. 45-9.
137. Arif, S., et al., GAD-alum immunotherapy in type 1 diabetes expands bifunctional Th1/Th2 autoreactive CD4 T cells. Diabetologia, 2020. 63(6): p. 1186-1198.
138. Ludvigsson, J., GAD-alum (Diamyd)—a new concept for preservation of residual insulin secretion. Expert Opin Biol Ther, 2010. 10(5): p. 787-99.
139. Hannelius, U., C. A. Beam, and J. Ludvigsson, Efficacy of GAD-alum immunotherapy associated with HLA-DR3-DQ2 in recently diagnosed type 1 diabetes. Diabetologia, 2020. 63(10): p. 2177-2181.
140. Faustman, D. L., et al., Proof-of-concept, randomized, controlled clinical trial of Bacillus-Calmette-Guerin for treatment of long-term type 1 diabetes. PLoS One, 2012. 7(8): p. e41756.
141. Ludvigsson, J., Autoantigen Treatment in Type 1 Diabetes: Unsolved Questions on How to Select Autoantigen and Administration Route. Int J Mol Sci, 2020. 21(5).
142. Tavira, B., et al., Intralymphatic Glutamic Acid Decarboxylase-Alum Administration Induced Th2-Like-Specific Immunomodulation in Responder Patients: A Pilot Clinical Trial in Type 1 Diabetes. J Diabetes Res, 2018. 2018: p. 9391845.
143. Casas, R., et al., Glutamic Acid Decarboxylase Injection Into Lymph Nodes: Beta Cell Function and Immune Responses in Recent Onset Type 1 Diabetes Patients. Front Immunol, 2020. 11: p. 564921.
144. Ludvigsson, J., et al., Intralymphatic Glutamic Acid Decarboxylase With Vitamin D Supplementation in Recent-Onset Type 1 Diabetes: A Double-Blind, Randomized, Placebo-Controlled Phase IIb Trial. Diabetes Care, 2021.
145. Ludvigsson, J., et al., Combined Etanercept, GAD-alum and vitamin D treatment: an open pilot trial to preserve beta cell function in recent onset type 1 diabetes. Diabetes Metab Res Rev, 2021: p. e3440.
146. Pihl, M., et al., Regulatory T cell phenotype and function 4 years after GAD-alum treatment in children with type 1 diabetes. Clin Exp Immunol, 2013. 172(3): p. 394-402.
147. Sinha, P., et al., Prostaglandin E2 promotes tumor progression by inducing myeloid-derived suppressor cells. Cancer Res, 2007. 67(9): p. 4507-13.
148. Fricke, I., et al., Vascular endothelial growth factor-trap overcomes defects in dendritic cell differentiation but does not improve antigen-specific immune responses. Clin Cancer Res, 2007. 13(16): p. 4840-8.
149. Talmadge, J. E., et al., Myelostimulatory activity of recombinant human interleukin-2 in mice. Blood, 1989. 73(6): p. 1458-67.
150. Nestle, F. O., et al., Vaccination of melanoma patients with peptide-or tumor lysate-pulsed dendritic cells. Nat Med, 1998. 4(3): p. 328-32.
151. Chakraborty, N. G., et al., Immunization with a tumor-cell-lysate-loaded autologous-antigen-presenting-cell-based vaccine in melanoma. Cancer Immunol Immunother, 1998. 47(1): p. 58-64.
152. Wang, F., et al., Phase I trial of a MART-1 peptide vaccine with incomplete Freund's adjuvant for resected high-risk melanoma. Clin Cancer Res, 1999. 5(10): p. 2756-65.
153. Thurner, B., et al., Vaccination with mage-3A1 peptide-pulsed mature, monocyte-derived dendritic cells expands specific cytotoxic T cells and induces regression of some metastases in advanced stage IV melanoma. J Exp Med, 1999. 190(11): p. 1669-78.
154. Thomas, R., et al., Immature human monocyte-derived dendritic cells migrate rapidly to draining lymph nodes after intradermal injection for melanoma immunotherapy. Melanoma Res, 1999. 9(5): p. 474-81.
155. Mackensen, A., et al., Phase I study in melanoma patients of a vaccine with peptide-pulsed dendritic cells generated in vitro from CD34(+) hematopoietic progenitor cells. Int J Cancer, 2000. 86(3): p. 385-92.
156. Panelli, M. C., et al., Phase I study in patients with metastatic melanoma of immunization with dendritic cells presenting epitopes derived from the melanoma-associated antigens MART-1 and gp100. J Immunother, 2000. 23(4): p. 487-98.
157. Schuler-Thurner, B., et al., Mage-3 and influenza-matrix peptide-specific cytotoxic T cells are inducible in terminal stage HLA-A2.1+ melanoma patients by mature monocyte-derived dendritic cells. J Immunol, 2000. 165(6): p. 3492-6.
158. Lau, R., et al., Phase I trial of intravenous peptide-pulsed dendritic cells in patients with metastatic melanoma. J Immunother, 2001. 24(1): p. 66-78.
159. Banchereau, J., et al., Immune and clinical responses in patients with metastatic melanoma to CD34(+) progenitor-derived dendritic cell vaccine. Cancer Res, 2001. 61(17): p. 6451-8.
160. Schuler-Thurner, B., et al., Rapid induction of tumor-specific type 1T helper cells in metastatic melanoma patients by vaccination with mature, cryopreserved, peptide-loaded monocyte-derived dendritic cells. J Exp Med, 2002. 195(10): p. 1279-88.
161. Palucka, A. K., et al., Single injection of CD34+ progenitor-derived dendritic cell vaccine can lead to induction of T-cell immunity in patients with stage IV melanoma. J Immunother, 2003. 26(5): p. 432-9.
162. Bedrosian, I., et al., Intranodal administration of peptide-pulsed mature dendritic cell vaccines results in superior CD8+T-cell function in melanoma patients. J Clin Oncol, 2003. 21(20): p. 3826-35.
163. Slingluff, C. L., Jr., et al., Clinical and immunologic results of a randomized phase II trial of vaccination using four melanoma peptides either administered in granulocyte-macrophage colony-stimulating factor in adjuvant or pulsed on dendritic cells. J Clin Oncol, 2003. 21(21): p. 4016-26.
164. Hersey, P., et al., Phase I/II study of treatment with dendritic cell vaccines in patients with disseminated melanoma. Cancer Immunol Immunother, 2004. 53(2): p. 125-34.
165. Vilella, R., et al., Pilot study of treatment of biochemotherapy-refractory stage IV melanoma patients with autologous dendritic cells pulsed with a heterologous melanoma cell line lysate. Cancer Immunol Immunother, 2004. 53(7): p. 651-8.
166. Palucka, A. K., et al., Spontaneous proliferation and type 2 cytokine secretion by CD4+ T cells in patients with metastatic melanoma vaccinated with antigen-pulsed dendritic cells. J Clin Immunol, 2005. 25(3): p. 288-95.
167. Banchereau, J., et al., Immune and clinical outcomes in patients with stage IV melanoma vaccinated with peptide-pulsed dendritic cells derived from CD34+ progenitors and activated with type I interferon. J Immunother, 2005. 28(5): p. 505-16.
168. Trakatelli, M., et al., A new dendritic cell vaccine generated with interleukin-3 and interferon-beta induces CD8+ T cell responses against NA17-A2 tumor peptide in melanoma patients. Cancer Immunol Immunother, 2006. 55(4): p. 469-74.
169. Salcedo, M., et al., Vaccination of melanoma patients using dendritic cells loaded with an allogeneic tumor cell lysate. Cancer Immunol Immunother, 2006. 55(7): p. 819-29.
170. Linette, G. P., et al., Immunization using autologous dendritic cells pulsed with the melanoma-associated antigen gp100-derived G280-9V peptide elicits CD8+ immunity. Clin Cancer Res, 2005. 11(21): p. 7692-9.
171. Escobar, A., et al., Dendritic cell immunizations alone or combined with low doses of interleukin-2 induce specific immune responses in melanoma patients. Clin Exp Immunol, 2005. 142(3): p. 555-68.
172. Tuettenberg, A., et al., Induction of strong and persistent MelanA/MART-1-specific immune responses by adjuvant dendritic cell-based vaccination of stage II melanoma patients. Int J Cancer, 2006. 118(10): p. 2617-27.
173. Schadendorf, D., et al., Dacarbazine (DTIC) versus vaccination with autologous peptide-pulsed dendritic cells (DC) in first-line treatment of patients with metastatic melanoma: a randomized phase III trial of the DC study group of the DeCOG. Ann Oncol, 2006. 17(4): p. 563-70.
174. Di Pucchio, T., et al., Immunization of stage IV melanoma patients with Melan-A/MART-1 and gp100 peptides plus IFN-alpha results in the activation of specific CD8(+) T cells and monocyte/dendritic cell precursors. Cancer Res, 2006. 66(9): p. 4943-51.
175. Nakai, N., et al., Vaccination of Japanese patients with advanced melanoma with peptide, tumor lysate or both peptide and tumor lysate-pulsed mature, monocyte-derived dendritic cells. J Dermatol, 2006. 33(7): p. 462-72.
176. Palucka, A. K., et al., Dendritic cells loaded with killed allogeneic melanoma cells can induce objective clinical responses and MART-1 specific CD8+T-cell immunity. J Immunother, 2006. 29(5): p. 545-57.
177. Lesimple, T., et al., Immunologic and clinical effects of injecting mature peptide-loaded dendritic cells by intralymphatic and intranodal routes in metastatic melanoma patients. Clin Cancer Res, 2006. 12(24): p. 7380-8.
178. Guo, J., et al., Intratumoral injection of dendritic cells in combination with local hyperthermia induces systemic antitumor effect in patients with advanced melanoma. Int J Cancer, 2007. 120(11): p. 2418-25.
179. O'Rourke, M. G., et al., Dendritic cell immunotherapy for stage IV melanoma. Melanoma Res, 2007. 17(5): p. 316-22.
180. Bercovici, N., et al., Analysis and characterization of antitumor T-cell response after administration of dendritic cells loaded with allogeneic tumor lysate to metastatic melanoma patients. J Immunother, 2008. 31(1): p. 101-12.
181. Hersey, P., et al., Phase I/II study of treatment with matured dendritic cells with or without low dose IL-2 in patients with disseminated melanoma. Cancer Immunol Immunother, 2008. 57(7): p. 1039-51.
182. von Euw, E. M., et al., A phase I clinical study of vaccination of melanoma patients with dendritic cells loaded with allogeneic apoptotic/necrotic melanoma cells. Analysis of toxicity and immune response to the vaccine and of IL-10-1082 promoter genotype as predictor of disease progression. J Transl Med, 2008. 6: p. 6.
183. Carrasco, J., et al., Vaccination of a melanoma patient with mature dendritic cells pulsed with MAGE-3 peptides triggers the activity of nonvaccine anti-tumor cells. J Immunol, 2008. 180(5): p. 3585-93.
184. Redman, B. G., et al., Phase Ib trial assessing autologous, tumor-pulsed dendritic cells as a vaccine administered with or without IL-2 in patients with metastatic melanoma. J Immunother, 2008. 31(6): p. 591-8.
185. Daud, A. I., et al., Phenotypic and functional analysis of dendritic cells and clinical outcome in patients with high-risk melanoma treated with adjuvant granulocyte macrophage colony-stimulating factor. J Clin Oncol, 2008. 26(19): p. 3235-41.
186. Engell-Noerregaard, L., et al., Review of clinical studies on dendritic cell-based vaccination of patients with malignant melanoma: assessment of correlation between clinical response and vaccine parameters. Cancer Immunol Immunother, 2009. 58(1): p. 1-14.
187. Nakai, N., et al., Immunohistological analysis of peptide-induced delayed-type hypersensitivity in advanced melanoma patients treated with melanoma antigen-pulsed mature monocyte-derived dendritic cell vaccination. J Dermatol Sci, 2009. 53(1): p. 40-7.
188. Dillman, R. O., et al., Phase II trial of dendritic cells loaded with antigens from self-renewing, proliferating autologous tumor cells as patient-specific antitumor vaccines in patients with metastatic melanoma: final report. Cancer Biother Radiopharm, 2009. 24(3): p. 311-9.
189. Chang, J. W., et al., Immunotherapy with dendritic cells pulsed by autologous dactinomycin-induced melanoma apoptotic bodies for patients with malignant melanoma. Melanoma Res, 2009. 19(5): p. 309-15.
190. Trepiakas, R., et al., Vaccination with autologous dendritic cells pulsed with multiple tumor antigens for treatment of patients with malignant melanoma: results from a phase I/II trial. Cytotherapy, 2010. 12(6): p. 721-34.
191. Jacobs, J. F., et al., Dendritic cell vaccination in combination with anti-CD25 monoclonal antibody treatment: a phase I/II study in metastatic melanoma patients. Clin Cancer Res, 2010. 16(20): p. 5067-78.
192. Ribas, A., et al., Multicenter phase II study of matured dendritic cells pulsed with melanoma cell line lysates in patients with advanced melanoma. J Transl Med, 2010. 8: p. 89.
193. Ridolfi, L., et al., Unexpected high response rate to traditional therapy after dendritic cell-based vaccine in advanced melanoma: update of clinical outcome and subgroup analysis. Clin Dev Immunol, 2010. 2010: p. 504979.
194. Cornforth, A. N., et al., Resistance to the proapoptotic effects of interferon-gamma on melanoma cells used in patient-specific dendritic cell immunotherapy is associated with improved overall survival. Cancer Immunol Immunother, 2011. 60(1): p. 123-31.
195. Lesterhuis, W. J., et al., Wild-type and modified gp100 peptide-pulsed dendritic cell vaccination of advanced melanoma patients can lead to long-term clinical responses independent of the peptide used. Cancer Immunol Immunother, 2011. 60(2): p. 249-60.
196. Bjoern, J., et al., Changes in peripheral blood level of regulatory T cells in patients with malignant melanoma during treatment with dendritic cell vaccination and low-dose IL-2. Scand J Immunol, 2011. 73(3): p. 222-33.
197. Steele, J. C., et al., Phase I/II trial of a dendritic cell vaccine transfected with DNA encoding melon A and gp100 for patients with metastatic melanoma. Gene Ther, 2011. 18(6): p. 584-93.
198. Kim, D. S., et al., Immunotherapy of malignant melanoma with tumor lysate-pulsed autologous monocyte-derived dendritic cells. Yonsei Med J, 2011. 52(6): p. 990-8.
199. Ellebaek, E., et al., Metastatic melanoma patients treated with dendritic cell vaccination, Interleukin-2 and metronomic cyclophosphamide: results from a phase II trial. Cancer Immunol Immunother, 2012. 61(10): p. 1791-804.
200. Dillman, R. O., et al., Tumor stem cell antigens as consolidative active specific immunotherapy: a randomized phase II trial of dendritic cells versus tumor cells in patients with metastatic melanoma. J Immunother, 2012. 35(8): p. 641-9.
201. Dannull, J., et al., Melanoma immunotherapy using mature DCs expressing the constitutive proteasome. J Clin Invest, 2013. 123(7): p. 3135-45.
202. Finkelstein, S. E., et al., Combination of external beam radiotherapy (EBRT) with intratumoral injection of dendritic cells as neo-adjuvant treatment of high-risk soft tissue sarcoma patients. Int J Radiat Oncol Biol Phys, 2012. 82(2): p. 924-32.
203. Stift, A., et al., Dendritic cell vaccination in medullary thyroid carcinoma. Clin Cancer Res, 2004. 10(9): p. 2944-53.
204. Kuwabara, K., et al., Results of a phase I clinical study using dendritic cell vaccinations for thyroid cancer. Thyroid, 2007. 17(1): p. 53-8.
205. Bachleitner-Hofmann, T., et al., Pilot trial of autologous dendritic cells loaded with tumor lysate(s) from allogeneic tumor cell lines in patients with metastatic medullary thyroid carcinoma. Oncol Rep, 2009. 21(6): p. 1585-92.
206. Yu, J. S., et al., Vaccination of malignant glioma patients with peptide-pulsed dendritic cells elicits systemic cytotoxicity and intracranial T-cell infiltration. Cancer Res, 2001. 61(3): p. 842-7.
207. Yamanaka, R., et al., Vaccination of recurrent glioma patients with tumour lysate-pulsed dendritic cells elicits immune responses: results of a clinical phase I/II trial. Br J Cancer, 2003. 89(7): p. 1172-9.
208. Yu, J. S., et al., Vaccination with tumor lysate-pulsed dendritic cells elicits antigen-specific, cytotoxic T-cells in patients with malignant glioma. Cancer Res, 2004. 64(14): p. 4973-9.
209. Yamanaka, R., et al., Tumor lysate and IL-18 loaded dendritic cells elicits Th1 response, tumor-specific CD8+ cytotoxic T cells in patients with malignant glioma. J Neurooncol, 2005. 72(2): p. 107-13.
210. Yamanaka, R., et al., Clinical evaluation of dendritic cell vaccination for patients with recurrent glioma: results of a clinical phase VII trial. Clin Cancer Res, 2005. 11(11): p. 4160-7.
211. Liau, L. M., et al., Dendritic cell vaccination in glioblastoma patients induces systemic and intracranial T-cell responses modulated by the local central nervous system tumor microenvironment. Clin Cancer Res, 2005. 11(15): p. 5515-25.
212. Walker, D. G., et al., Results of a phase I dendritic cell vaccine trial for malignant astrocytoma: potential interaction with adjuvant chemotherapy. J Clin Neurosci, 2008. 15(2): p. 114-21.
213. Leplina, O. Y., et al., Use of interferon-alpha-induced dendritic cells in the therapy of patients with malignant brain gliomas. Bull Exp Biol Med, 2007. 143(4): p. 528-34.
214. De Vleeschouwer, S., et al., Postoperative adjuvant dendritic cell-based immunotherapy in patients with relapsed glioblastoma multiforme. Clin Cancer Res, 2008. 14(10): p. 3098-104.
215. Ardon, H., et al., Adjuvant dendritic cell-based tumour vaccination for children with malignant brain tumours. Pediatr Blood Cancer, 2010. 54(4): p. 519-25.
216. Prins, R. M., et al., Gene expression profile correlates with T-cell infiltration and relative survival in glioblastoma patients vaccinated with dendritic cell immunotherapy. Clin Cancer Res, 2011. 17(6): p. 1603-15.
217. Okada, H., et al., Induction of CDS+T-cell responses against novel glioma-associated antigen peptides and clinical activity by vaccinations with {alpha}-type 1 polarized dendritic cells and polyinosinic-polycytidylic acid stabilized by lysine and carboxymethylcellulose in patients with recurrent malignant glioma. J Clin Oncol, 2011. 29(3): p. 330-6.
218. Fadul, C. E., et al., Immune response in patients with newly diagnosed glioblastoma multiforme treated with intranodal autologous tumor lysate-dendritic cell vaccination after radiation chemotherapy. J Immunother, 2011. 34(4): p. 382-9.
219. Chang, C. N., et al., A phase I/II clinical trial investigating the adverse and therapeutic effects of a postoperative autologous dendritic cell tumor vaccine in patients with malignant glioma. J Clin Neurosci, 2011. 18(8): p. 1048-54.
220. Cho, D. Y., et al., Adjuvant immunotherapy with whole-cell lysate dendritic cells vaccine for glioblastoma multiforme: a phase II clinical trial. World Neurosurg, 2012. 77(5-6): p. 736-44.
221. Iwami, K., et al., Peptide-pulsed dendritic cell vaccination targeting interleukin-13 receptor alpha2 chain in recurrent malignant glioma patients with HLA-A*24/A*02 allele. Cytotherapy, 2012. 14(6): p. 733-42.
222. Fong, B., et al., Monitoring of regulatory T cell frequencies and expression of CTLA-4 on T cells, before and after DC vaccination, can predict survival in GBM patients. PLoS One, 2012. 7(4): p. e32614.
223. De Vleeschouwer, S., et al., Stratification according to HGG-IMMUNO RPA model predicts outcome in a large group of patients with relapsed malignant glioma treated by adjuvant postoperative dendritic cell vaccination. Cancer Immunol Immunother, 2012. 61(11): p. 2105-12.
224. Phuphanich, S., et al., Phase I trial of a multi-epitope-pulsed dendritic cell vaccine for patients with newly diagnosed glioblastoma. Cancer Immunol Immunother, 2013. 62(1): p. 125-35.
225. Akiyama, Y., et al., alpha-type-1 polarized dendritic cell-based vaccination in recurrent high-grade glioma: a phase I clinical trial. BMC Cancer, 2012. 12: p. 623.
226. Prins, R. M., et al., Comparison of glioma-associated antigen peptide-loaded versus autologous tumor lysate-loaded dendritic cell vaccination in malignant glioma patients. J Immunother, 2013. 36(2): p. 152-7.
227. Shah, A. H., et al., Dendritic cell vaccine for recurrent high-grade gliomas in pediatric and adult subjects: clinical trial protocol. Neurosurgery, 2013. 73(5): p. 863-7.
228. Reichardt, V. L., et al., Idiotype vaccination using dendritic cells after autologous peripheral blood stem cell transplantation for multiple myeloma—a feasibility study. Blood, 1999. 93(7): p. 2411-9.
229. Um, S. H. and R. Bailey-Wood, Idiotypic protein-pulsed dendritic cell vaccination in multiple myeloma. Int J Cancer, 1999. 83(2): p. 215-22.
230. Motta, M. R., et al., Generation of dendritic cells from CD14+ monocytes positively selected by immunomagnetic adsorption for multiple myeloma patients enrolled in a clinical trial of anti-idiotype vaccination. Br J Haematol, 2003. 121(2): p. 240-50.
231. Reichardt, V. L., et al., Idiotype vaccination of multiple myeloma patients using monocyte-derived dendritic cells. Haematologica, 2003. 88(10): p. 1139-49.
232. Guardino, A. E., et al., Production of myeloid dendritic cells (DC) pulsed with tumor-specific idiotype protein for vaccination of patients with multiple myeloma. Cytotherapy, 2006. 8(3): p. 277-89.
233. Lacy, M. Q., et al., Idiotype-pulsed antigen-presenting cells following autologous transplantation for multiple myeloma may be associated with prolonged survival. Am J Hematol, 2009. 84(12): p. 799-802.
234. Yi, Q., et al., Optimizing dendritic cell-based immunotherapy in multiple myeloma: intranodal injections of idiotype-pulsed CD40 ligand-matured vaccines led to induction of type-1 and cytotoxic T-cell immune responses in patients. Br J Haematol, 2010. 150(5): p. 554-64.
235. Rollig, C., et al., Induction of cellular immune responses in patients with stage-I multiple myeloma after vaccination with autologous idiotype-pulsed dendritic cells. J Immunother, 2011. 34(1): p. 100-6.
236. Zahradova, L., et al., Efficacy and safety of Id-protein-loaded dendritic cell vaccine in patients with multiple myeloma—phase II study results. Neoplasma, 2012. 59(4): p. 440-9.
237. Timmerman, J. M., et al., Idiotype-pulsed dendritic cell vaccination for B-cell lymphoma: clinical and immune responses in 35 patients. Blood, 2002. 99(5): p. 1517-26.
238. Maier, T., et al., Vaccination of patients with cutaneous T-cell lymphoma using intranodal injection of autologous tumor-lysate-pulsed dendritic cells. Blood, 2003. 102(7): p. 2338-44.
239. Di Nicola, M., et al., Vaccination with autologous tumor-loaded dendritic cells induces clinical and immunologic responses in indolent B-cell lymphoma patients with relapsed and measurable disease: a pilot study. Blood, 2009. 113(1): p. 18-27.
240. Hus, I., et al., Allogeneic dendritic cells pulsed with tumor lysates or apoptotic bodies as immunotherapy for patients with early-stage B-cell chronic lymphocytic leukemia. Leukemia, 2005. 19(9): p. 1621-7.
241. Li, L., et al., Immunotherapy for patients with acute myeloid leukemia using autologous dendritic cells generated from leukemic blasts. Int J Oncol, 2006. 28(4): p. 855-61.
242. Roddie, H., et al., Phase VII study of vaccination with dendritic-like leukaemia cells for the immunotherapy of acute myeloid leukaemia. Br J Haematol, 2006. 133(2): p. 152-7.
243. Litzow, M. R., et al., Testing the safety of clinical-grade mature autologous myeloid DC in a phase I clinical immunotherapy trial of CML. Cytotherapy, 2006. 8(3): p. 290-8.
244. Westermann, J., et al., Vaccination with autologous non-irradiated dendritic cells in patients with bcr/abl+ chronic myeloid leukaemia. Br J Haematol, 2007. 137(4): p. 297-306.
245. Hus, I., et al., Vaccination of 8-CLL patients with autologous dendritic cells can change the frequency of leukemia antigen-specific CD8+ T cells as well as CD4+CD25+FoxP3+ regulatory T cells toward an antileukemia response. Leukemia, 2008. 22(5): p. 1007-17.
246. Palma, M., et al., Development of a dendritic cell-based vaccine for chronic lymphocytic leukemia. Cancer Immunol Immunother, 2008. 57(11): p. 1705-10.
247. Van Tendeloo, V. F., et al., Induction of complete and molecular remissions in acute myeloid leukemia by Wilms' tumor 1 antigen-targeted dendritic cell vaccination. Proc Natl Acad Sci USA, 2010. 107(31): p. 13824-9.
248. Iwashita, Y., et al., A phase I study of autologous dendritic cell-based immunotherapy for patients with unresectable primary liver cancer. Cancer Immunol Immunother, 2003. 52(3): p. 155-61.
249. Lee, W. C., et al., Vaccination of advanced hepatocellular carcinoma patients with tumor lysate-pulsed dendritic cells: a clinical trial. J Immunother, 2005. 28(5): p. 496-504.
250. Butterfield, L. H., et al., A phase VII trial testing immunization of hepatocellular carcinoma patients with dendritic cells pulsed with four alpha-fetoprotein peptides. Clin Cancer Res, 2006. 12(9): p. 2817-25.
251. Palmer, D. H., et al., A phase II study of adoptive immunotherapy using dendritic cells pulsed with tumor lysate in patients with hepatocellular carcinoma. Hepatology, 2009. 49(1): p. 124-32.
252. El Ansary, M., et al., Immunotherapy by autologous dendritic cell vaccine in patients with advanced HCC. J Cancer Res Clin Oncol, 2013. 139(1): p. 39-48.
253. Tada, F., et al., Phase VII study of immunotherapy using tumor antigen-pulsed dendritic cells in patients with hepatocellular carcinoma. Int J Oncol, 2012. 41(5): p. 1601-9.
254. Ueda, Y., et al., Dendritic cell-based immunotherapy of cancer with carcinoembryonic antigen-derived, HLA-A24-restricted CTL epitope: Clinical outcomes of 18 patients with metastatic gastrointestinal or lung adenocarcinomas. Int J Oncol, 2004. 24(4): p. 909-17.
255. Hirschowitz, E. A., et al., Autologous dendritic cell vaccines for non-small-cell lung cancer. J Clin Oncol, 2004. 22(14): p. 2808-15.
256. Chang, G. C., et al., A pilot clinical trial of vaccination with dendritic cells pulsed with autologous tumor cells derived from malignant pleural effusion in patients with late-stage lung carcinoma. Cancer, 2005. 103(4): p. 763-71.
257. Yannelli, J. R., et al., The large scale generation of dendritic cells for the immunization of patients with non-small cell lung cancer (NSCLC). Lung Cancer, 2005. 47(3): p. 337-50.
258. Ishikawa, A., et al., A phase I study of alpha-galactosylceramide (KRN7000)-pulsed dendritic cells in patients with advanced and recurrent non-small cell lung cancer. Clin Cancer Res, 2005. 11(5): p. 1910-7.
259. Antonia, S. J., et al., Combination of p53 cancer vaccine with chemotherapy in patients with extensive stage small cell lung cancer. Clin Cancer Res, 2006. 12(3 Pt 1): p. 878-87.
260. Perrot, I., et al., Dendritic cells infiltrating human non-small cell lung cancer are blocked at immature stage. J Immunol, 2007. 178(5): p. 2763-9.
261. Hirschowitz, E. A., et al., Immunization of NSCLC patients with antigen-pulsed immature autologous dendritic cells. Lung Cancer, 2007. 57(3): p. 365-72.
262. Baratelli, F., et al., Pre-clinical characterization of GMP grade CCL21-gene modified dendritic cells for application in a phase I trial in non-small cell lung cancer. J Transl Med, 2008. 6: p. 38.
263. Hegmans, J. P., et al., Consolidative dendritic cell-based immunotherapy elicits cytotoxicity against malignant mesothelioma. Am J Respir Crit Care Med, 2010. 181(12): p. 1383-90.
264. Um, S. J., et al., Phase I study of autologous dendritic cell tumor vaccine in patients with non-small cell lung cancer. Lung Cancer, 2010. 70(2): p. 188-94.
265. Chiappori, A. A., et al., INGN-225: a dendritic cell-based p53 vaccine (Ad.p53-DC) in small cell lung cancer: observed association between immune response and enhanced chemotherapy effect. Expert Opin Biol Ther, 2010. 10(6): p. 983-91.
266. Perroud, M. W., Jr., et al., Mature autologous dendritic cell vaccines in advanced non-small cell lung cancer: a phase I pilot study. J Exp Clin Cancer Res, 2011. 30: p. 65.
267. Skachkova, O. V., et al., Immunological markers of anti-tumor dendritic cells vaccine efficiency in patients with non-small cell lung cancer. Exp Oncol, 2013. 35(2): p. 109-13.
268. Hernando, J. J., et al., Vaccination with autologous tumour antigen-pulsed dendritic cells in advanced gynaecological malignancies: clinical and immunological evaluation of a phase I trial. Cancer Immunol Immunother, 2002. 51(1): p. 45-52.
269. Rahma, O. E., et al., A gynecologic oncology group phase II trial of two p53 peptide vaccine approaches: subcutaneous injection and intravenous pulsed dendritic cells in high recurrence risk ovarian cancer patients. Cancer Immunol Immunother, 2012. 61(3): p. 373-84.
270. Chu, C. S., et al., Phase VII randomized trial of dendritic cell vaccination with or without cyclophosphamide for consolidation therapy of advanced ovarian cancer in first or second remission. Cancer Immunol Immunother, 2012. 61(5): p. 629-41.
271. Kandalaft, L. E., et al., A Phase I vaccine trial using dendritic cells pulsed with autologous oxidized lysate for recurrent ovarian cancer. J Transl Med, 2013. 11: p. 149.
272. Lepisto, A. J., et al., A phase VII study of a MUC1 peptide pulsed autologous dendritic cell vaccine as adjuvant therapy in patients with resected pancreatic and biliary tumors. Cancer Ther, 2008. 6(B): p. 955-964.
273. Rong, Y., et al., A phase I pilot trial of MUC1-peptide-pulsed dendritic cells in the treatment of advanced pancreatic cancer. Clin Exp Med, 2012. 12(3): p. 173-80.
274. Endo, H., et al., Phase I trial of preoperative intratumoral injection of immature dendritic cells and OK-432 for resectable pancreatic cancer patients. J Hepatobiliary Pancreat Sci, 2012. 19(4): p. 465-75.
275. Zhao, T. X., et al., Low-dose interleukin-2 in patients with stable ischaemic heart disease and acute coronary syndromes (LILACS): protocol and study rationale for a randomised, double-blind, placebo-controlled, phase VII clinical trial. BMJ Open, 2018. 8(9): p. e022452.

1. A method of generating a monocytic lineage cell capable of suppressing inflammation, comprising: a) obtaining a monocytic population; b) culturing said monocytic cell population in media conditioned by regenerative cells; and c) optionally adding factors capable of supporting/synergizing with regenerative cells at inducing generation of inflammation suppressing myeloid cells.

2. The method of claim 1, wherein said cell possesses ability to induce angiogenesis.

3. The method of claim 1, wherein said cell possesses ability to induce neurogenesis.

4. The method of claim 1, wherein said cell possesses ability to induce activation of endogenous regenerative cells.

5. The method of claim 1, were said regenerative cells are mesenchymal stem cells.

6. The method of claim 1, wherein said monocytic cell population is isolated by positive selection for cells expressing GR-1.

7. The method of claim 1, wherein said monocytic cell population is isolated by positive selection for cells expressing interferon gamma receptor.

8. The method of claim 1, wherein said monocytic cell population is isolated by positive selection for cells expressing interleukin-4 receptor.

9. The method of claim 1, wherein said monocytic cell population is isolated by positive selection for cells expressing interleukin-13 receptor.

10. The method of claim 1, wherein said monocytic cell population is isolated by positive selection for cells expressing interleukin-10 receptor.

11. The method of claim 1, wherein said monocytic cell population is isolated by positive selection for cells expressing HLA-DR.

12. The method of claim 1, wherein said monocytic cell population is isolated by positive selection for cells expressing TGF-beta receptor.

13. The method of claim 5, wherein said mesenchymal stem cells are generated from pluripotent stem cells by a) culturing single cells in the presence of at least one growth factor in an amount sufficient to induce the differentiation of said clusters of cells into mesenchymal stem cells; b) adding one or more of basic fibroblast growth factor (bFGF), vascular endothelial growth factor (VEGF), bone morphogenic protein 4 (BMP-4), stem cell factor (SCF), Flt 3L (FL), thrombopoietin (TPO), EPO, and/or tPTD-HOXB4. The one or more of said at least one growth factor added in step (b) may be added to said culture within 36-60 hours from the start of step (a). Preferably, the one or more of said at least one growth factor added in step (b) is added to said culture within 40-48 hours from the start of step (a). The at least one factor added in step (b) may comprise one or more of bFGF, VEGF, BMP-4, SCF, FL and/or tPTD-HOXB4. The concentration of said growth factors if added in step (b) may range from about the following: bFGF is about 20-25 ng/ml, VEGF is about 20-100 ng/ml, BMP-4 is about 15-100 ng/ml, SCF is about 20-50 ng/ml, FL is about 10-50 ng/ml, TPO is about 20-50 ng/ml, and tPTD-HOXB4 is about 1.5-5 U/ml.

14. The method of claim 1, wherein said regenerative cells are subepithelial umbilical cord derived regenerative cells.

15. The method of claim 14, wherein said subepithelial cord tissue stem cells are prepared by a process comprising: placing a subepithelial layer of a mammalian umbilical cord tissue in direct contact with a growth substrate; and culturing the subepithelial layer such that the isolated cell from the subepithelial layer is capable of self-renewal and culture expansion, wherein the isolated cell expresses at least three cell markers selected from the group consisting of CD29, CD73, CD90, CD166, SSEA4, CD9, CD44, CD146, or CD105, and wherein the isolated cell does not express NANOG and at least five cell markers selected from the group consisting of CD45, CD34, CD14, CD79, CD106, CD86, CD80, CD19, CD117, Stro-1, or HLA-DR.

16. The method of claim 15, wherein said cells expresses CD29, CD73, CD90, CD166, SSEA4, CD9, CD44, CD146, and CD105.

17. The method of claim 16, wherein said cells express CD105.

18. The method of claim 17, wherein said cells express CD146.

19. The method of claim 18, wherein said cells express CD44.

20. The method of claim 19, wherein said cells express CD9.
